(19) United States
(12) Patent Application Publication
(10) Pub. No.: US 2025/0270295 A1
(43) Pub. Date: Aug. 28, 2025

(54) APPLICATION OF HCMV GP34- AND GP68-SPECIFIC ANTIBODIES AND FRAGMENTS THEREOF FOR PREVENTION, THERAPY AND DIAGNOSTICS OF HCMV DISEASE

(71) Applicants: Albert-Ludwigs-Universitaet Freiburg, Freiburg (DE); University of Rijeka, Faculty of Medicine, Rijeka (HR)

(72) Inventors: Philipp Leonhard KOLB, Freiburg (DE); Katja HOFFMAN, Freiburg (DE); Harmut HENGEL, Gundelfingen (DE); Stipan JONJIC, Rijeka (HR); Tihana LENAC-ROVIS, Kastav (HR)

(73) Assignees: Albert-Ludwigs-Universitaet Freiburg, Freiburg (DE); University of Rijeka, Faculty of Medicine, Rijeka (HR)

(21) Appl. No.: 18/571,406

(22) PCT Filed: Jun. 10, 2022
(86) PCT No.: PCT/EP2022/065800

§ 371 Date: Dec. 18, 2023

(30) Foreign Application Priority Data
Jun. 23, 2021 (EP) 21181202.9

(51) Int. Cl. C07K 16/08; A61P 31/22; G01N 33/569

(52) U.S. Cl. CPC C07K 16/089; A61P 31/22; G01N 33/56994; C07K 2317/24; C07K 2317/54; C07K 2317/565; C07K 2317/76; G01N 2333/045

(57) ABSTRACT

The present application relates to antibodies which bind to the extracellular domain of an Fc-gamma-binding glycoprotein gp34 and gp68 of human cytomegalovirus and its use in the treatment of a HCMV infection or in its prevention.

### PRIORITY

This application corresponds to the U.S. National phase of International Application No. PCT/EP2022/065800, filed Jun. 10, 2022, which, in turn, claims priority to European Patent Application No. 21181202.9 filed Jun. 23, 2021, the contents of which are incorporated by reference herein in their entirety.

### GRANT SUPPORT

This patent application was partially supported by the grant “Strengthening the capacity of CerVirVac for research in virus immunology and vaccinology”, KK.01.1.1.01.0006, awarded to the Scientific Centre of Excellence for Virus Immunology and Vaccines and co-financed by the European Regional Development Fund.

### SEQUENCE Listing

The instant application contains a Sequence Listing that has been submitted in ASCII format via EFS-Web and is hereby incorporated by reference in its entirety. Said ASCII copy, created on Jul. 11, 2024, is named LNK_253US_SL.txt and is 73,177 bytes in size.

### BACKGROUND OF THE PRESENT INVENTION

Treatment of CMV-related diseases and prevention of damage due to congenital CMV (cCMV) infection are one of the most pressing challenges of cytomegalovirus research. The US Institute of Medicine of the National Academy of Science declared the development of a vaccine to prevent cCMV as one of the highest priorities. Due to the high health impact of CMV disease, many research consortia and pharmaceutical companies are actively pursuing the development of an efficient CMV vaccine.

### SUMMARY OF THE PRESENT INVENTION

As human cytomegalovirus (HCMV) is best known to efficiently evade immune responses including humoral immunity the HCMV encoded FcγR antagonists gp34 and gp68 were thought to be responsible for limiting the efficacy of endogenous and therapeutic IgG antibodies directed against HCMV antigens. gp34 and gp68 were identified as HCMV RL11/gp34 and UL119_118/gp68 encoded Fcγ-binding glycoproteins and their ability to mediate evasion from FcγR-dependent IgG effector functions was described. The experiments performed according to the present invention were usually made with the soluble gp34 (sgp34), the sequence thereof is shown in SEQ ID NO:1.

Interfering with IgG binding by gp34 and gp68 may thus restore FcγR-mediated immune responses (e.g. Antibody-dependent cellular cytotoxicity (ADCC) of NK-cells or Antibody-dependent cellular Phagocytosis (ADCP) of phagocytic cells). gp34 and gp68 are well-conserved among HCMV wild-type strains and patient isolates and make them perfect targets for therapeutic intervention. Utilizing this knowledge for gp34 and gp68 targeting first mouse monoclonal antibodies (mAbs) were generated to block their immunoevasive function(s) and restoring HCMV-IgG effector functions. Evidence is provided showing that gp34 interacts with additional human immune ligands, extending its spectrum for immunoevasive functions. On this basis, the generated mAbs to gp34 may gain an even broader therapeutic and diagnostic applicability.

In the first round of experiments parts of the glycoproteins gp34 and gp68 (only extracellular domain, ECD) were used for the generation of mouse monoclonal antibodies. The glycoprotein gp34 binds to the Fc part of human IgG1, IgG2, IgG3, IgG4, mouse IgG1, mouse IgG2a and mouse IgG3, rat IgG and rabbit IgG. For the immunization experiments two different proteins derived from gp34 were used.

The amino acid sequence having the designation sol gp34 wt (SEQ ID NO:1) was used for immunization.

The amino acid sequence ID NO:1 has the following sequence:

(SEQ ID NO: 1)
MQTYSTPLTLVIVTSLFLFTTQGSSSNAVEPTKKPLKLANYRATCEDRT
RTLVTRLNTSHHSWVWQRYDIYSRYMRRMPPLCIITDAYKETTRQGGAA
FACTRQYTGDVEAFYLIIHPRSFCRALETRRCFYPGPGRVVVTDSQEAD
RAIISDLKRQWSGKPIPNPLLGLDSTRTGHHHHHH

comprising:
a signal peptide; 1-23 aa=SP
an Ig-like domain (32-137 aa)
a minimal IgG-Fc binding domain (based on analysis using truncated gp34 variants): 1-140 aa truncation variant: 1-124 aa→lost its ability to bind IgG-Fc,
a V5 Tag (GKPIPNPLLGLDSTRTG) and
poly-His Tag (HHHHHH).

In addition thereto, a modified sequence from gp34 having the designation sol gp34mtrp (more precisely gp34 1-179 W65F) (SEQ ID NO:2) was used. This protein corresponds with the extracellular domain of gp34 wt having amino acid sequences 1-179 whereby, however, the amino acid tryptophan at position 65 (in the one letter code “W”) was changed to phenylalanine (in the one letter code “F”). The correct designation of the mutant is therefore W65F.

SEQ ID NO:2 has the following amino acid sequence:

(SEQ ID NO: 2)
MQTYSTPLTLVIVTSLFLFTTQGSSSNAVEPTKKPLKLANYRATCEDRT
RTLVTRLNTSHHSVVFQRYDIYSRYMRRMPPLCIITDAYKETTRQGGAA
FACTRQNLTLYNLTVKDTGVYLLQDQYTGDVEAFYLIIHPRSFCRALET
RRCFYPGPGRVVVTDSQEADRAIISDLKRQWSGKPIPNPLLGLDSTRTG
HHHHHH

with an SP (signal peptide; 1-23 aa),
an Ig-like domain (32-137 aa),
a minimal IgG-Fc binding domain (based on analysis using truncated gp34 variants): 1-140 aa truncation variant: 1-124 aa→lost its ability to bind IgG-Fc,
with mtrp position: W65F (Trp→Phe),
a V5 Tag (GKPIPNPLLGLDSTRTG) and
a His Tag (HHHHHH).

The purpose of using those two antigens sol gp34 wt and sol gp34 mtrp was that the binding of the gp34 wt is different from the binding properties of the mutant. gp34 wt binds to human IgG1, IgG2, IgG3, IgG4, to mouse IgG1, to mouse IgG2a and mouse IgG3 and to rat IgG and rabbit IgG. The mutated gp34 has impaired binding properties for human IgG binding, looses the binding capability to all mouse IgGs, which was important since the immunization with gp34 wt protein might not result in the generation of specific antibodies.

As a further antigen for the immunization of mice in order to produce monoclonal antibodies the antigen derived from glycoprotein 68 having the designation sol gp68 which corresponds to the extracellular domain of gp68 was used. The sequence of this protein is shown as SEQ ID NO:3.

SEQ ID NO:3 has the following amino acid sequence:

(SEQ ID NO: 3)
MCSVLAIALVVALLGDMHPGVKSSTTSAVTSPSNTTVTSTTSISTSNNV
TSAVTTTVQTSTSSASTSVIATTQKEGHLYTVNCEASYSHDQVSLNATC
KVILLNNTKNPDILSVTCYARTDCKGPFTQVGYLSAFPPDNEGKLHLSY
NATAQELLISGLRPQETTEYTCSFFSWGRHHNATWDLFTYPIYAVYGTR
LNATTMRVRVLLQEHEHCLLNGSSLYHPNSTVHLHQGNQLIPPWNISNV
TYNGQRLREFVFYLNGTYTVVRLHVQIAGRSFTTTYVFIKSDPLFEDGK
PIPNPLLGLDSTRTGHHHHHH

with
an SP (signal peptide; 1-23 aa),
an Ig-like domain (32-137 aa),
a V5 Tag (GKPIPNPLLGLDSTRTG) and
a poly His Tag (HHHHHH).

The present invention relates to antibodies or antigen binding fragments thereof which bind to the extracellular domain of an Fc-gamma-binding glycoprotein of human cytomegalovirus. In one preferred embodiment the antibody or antigen binding fragment thereof binds to the Fc-gamma-binding glycoprotein which is gp34. The sequence to which the antibody or antigen binding fragment binds is the Ig-like domain of SEQ ID NO:1. The Ig-like domain stretching from amino acid 32 to 137 is part of the extracellular domain of gp34.

It should be noted that the term “antibody or antigen binding fragment thereof” encompasses all derivatives of antibodies which may be used for therapeutic and/or diagnostic purposes such as for example single-chain antigen binding fragments or nanobodies.

In an alternative embodiment the antibody and/or antigen binding fragments thereof bind to the mutated sequence of gp34 having SEQ ID NO:2. The mutated sequence differs from the wild type sequence only in position 65 whereby the wild type has a tryptophan at amino acid position 65 and the mutated sequence has a phenylalanine at position 65. The two sequences were used in the course of the present invention in order to investigate the different functional parts of the glycoprotein gp34.

The preliminary experiments were performed with monoclonal antibodies derived from mice. It is, however, plainly evident that mouse antibodies cannot be used in therapy. It is possible to use mouse antibodies for diagnostic purposes, but when applied to human patients the human body develops antibodies directed against the mouse-specific structures.

In order to overcome those difficulties it is common practice to humanize the antibodies. For the humanization of antibodies which can be done by using suitable computer programs it is searched for human antibodies which have the highest similarity or homology to the effective mouse antibodies. The framework of such human antibodies is used and the CDRs are “transplanted” from the mouse antibodies into the human framework. Very often it is required to adapt the sequences in order to maintain the best binding properties. Therefore, it may be required that one or two of the CDR sequences are replaced in the course of humanization. The advantage of having good working mouse monoclonal antibodies is the knowledge which CDRs can be best used for the humanization.

It is therefore a preferred embodiment of the present invention that the humanized antibodies and/or antigen binding fragments thereof have a very high homology to the preferred amino acid sequences of the best working monoclonal antibodies. Preferably, the sequence homology or sequence identity is at least 80% with the mouse sequences disclosed herein. In more preferred embodiments the sequence identity or sequence homology is at least 85% and in especially preferred embodiments the sequence identity and/or sequence homology is at least 95% to the mouse sequences. The highest homology and/or identity is required in the CDR regions whereas in the framework areas a lower homology is acceptable. Other properties like the efficient expression in host cells or the further processability for pharmaceutical uses may require additional adaptations of the amino acid sequences. Such preferred sequences are in particular SEQ ID NO:14-SEQ ID NO:59.

The property of the antibodies and antigen binding fragments thereof is important as such antibodies are able to block the binding of the glycoprotein gp34 to the Fc part of the immunoglobulin-gamma (IgG). By this interaction the counter-defense mechanism of the human cytomegalovirus by antagonizing the antibodies of the host is substantially weakened. It was demonstrated that gp34 and gp68 of human cytomegalovirus bind to the Fc parts of immunoglobulin-gamma whereby the host defense mechanisms are impaired. When this defense mechanism of HCMV is inhibited by the antibodies according to the invention the immune defense of the patient infected with HCMV is substantially improved.

Another aspect of the present invention is that the antibodies and/or antigen binding fragments thereof block the binding of glycoprotein gp34 to C-reactive protein. C-reactive protein is a well-known mediator of the immune response of the host cell (patient). By blocking the usual function of glycoprotein gp34 with the help of an antibody or antigen binding fragment thereof according to the invention the immune reaction of the infected patient is substantially improved.

A further very surprising effect of the present invention is that the antibodies and/or antigen binding fragments thereof according to the invention are able to block binding to the neonatal Fc receptor. This is of utmost importance in the treatment of pregnant women when adverse effects of the virus to the fetus are to be avoided.

In an alternative embodiment of the present invention the antibodies and/or antigen binding fragments thereof bind to glycoprotein gp68.

In addition to the therapeutic uses as described above the antibodies and/or antigen binding fragments of the present invention can be used for diagnostic purposes. In such diagnostic purposes the antibody and/or antigen binding fragments thereof are reacted with biological samples derived from a potentially infected subject. Such samples can be cell fragments obtained by biopsy or from biological fluids like blood, serum, plasma, saliva or liquor.

In general, the antibody and/or antigen binding fragment thereof according to the invention is reacted with the biological sample in order to allow the binding of the antibodies to the antigen gp68 and/or gp34. Subsequently the unbound antibodies and/or antigen binding fragments thereof are removed usually by washing and thereafter the antigen/antibody complex is detected by label. Such a label can already be incorporated into the antibody and/or antigen binding fragment or it can be detected by a suitable detection means. When for example monoclonal mouse antibodies are used the detection can be achieved by using antibodies probably derived from rabbit or goat and directed against the Fc part of mouse antibodies.

### DETAILED DESCRIPTION OF THE PREFERRED EMBODIMENTS

The following mouse monoclonal antibodies were generated and characterized, the genes were sequenced and expressed to produce recombinant IgG:
8 mAbs raised against the ectodomain of gp68 (1-292aa) capable to block IgG binding by gp68
9 mAbs raised against the ectodomain (1-179aa) of gp34, some of which block the IgG-gp34 interaction
6 mAbs raised against the point-mutation W65F (position 65 Trp→Phe of the ectodomain (1-179aa) of gp34. The mAbs recognize sgp34wt protein but the mAbs have only minor or no effect on gp34-IgG binding but could prevent binding of further gp34 ligands (namely CRP and FcRn).

Antigen specificity of the generated mAbs for the immunizing antigen was approved by ELISA, flow cytometry, immunoprecipitation and Western Blot, resp., and specifically controlled for the contributing binding effects of Fcγ. Blocking activities of gp34 and gp68 by mAbs was assessed in FcγR reporter cell activation assays.

Specifically, all anti-gp68 mAbs detect gp68 in ELISA, whereas only two of them recognize gp68 in western blot (gp68.01 and gp68.02). All tested anti-gp68 mAbs exhibit a strong blocking capacity of Fcy binding to gp68 in a dose-dependent manner.

Two different antigens (SEQ ID NOs:1 and 2) were used to generate mAbs to gp34, resulting in two kinds of antibody groups. First, antibodies were raised against sgp34wt protein which shows a strong IgG-Fc binding and has a strong potential to antagonize human FcyR-activation, named sgp34wt mAbs (wt.07/wt.08/wt.09/wt.10/wt.11/wt.12/wt.13/wt.14 and wt.15).

Second antibodies raised against gp34 W65F (mutated sequence) which is impaired for Fcγ-binding and lacks antagonization potential of FcyR-activation, named gp34mtrp mAbs (mtrp.01/mtrp02/mtrp.03/mtrp.04/mtrp.05 and mtrp.06) were provided.

Both groups of mAbs detect immobilized sgp34wt by ELISA. In contrast, none of the mAbs resulting from the immunization using native sgp34wt protein is able to detect structurally affected gp34 W65F in ELISA, as well as in western blot.

All gp34 W65F mAbs specifically recognize gp34 W65F protein, as well as the sgp34wt protein.

With regard to the potential of our anti-gp34 mAbs to interfere and block IgG binding by vFcyR gp34, only selected mAbs of the anti-sgp34wt mAb group show strong blocking capacities in a dose-dependent manner:

wt.09: IC50 63 ng/ml, wt. 10: IC50 89 ng/ml, wt. 11: IC50 77 ng/ml, wt. 12: IC50 70 ng/ml, wt. 15: IC50 120 ng/ml.

Some antibodies have an intermediate effect on sgp34wt-IgG binding at least reducing up to 50% of binding by the vFcyR:

wt.07: IC50 447 ng/ml, wt.08: IC50 494 ng/ml, mtrp.03: IC50 152 ng/ml, mtrp.04: IC50 147 ng/ml, mtrp.01: IC50 239 ng/ml; mtrp.02: IC50 330 ng/ml.

In contrast, IC50 values of mtrp.06 are IC50 4456 ng/ml and of mouse IgG1 isotype control IC50 9690 ng/ml.

To validate the functionality and blocking capacity of the mAbs using recombinantly generated Fab-fragments Fab-genes were sequenced and analyzed including framework (FR) and complementary-determining regions (CDRs). Furthermore, humanized Fab sequences were generated from selected, promising mAb candidates. Those might be further optimized by glyco-engineering and/or Fc-modifications addressing beneficial immune-reactions and avoiding unwanted actions. Also, half-life of humanized mAbs might be modified by introducing mutations within the Fc-region.

Besides IgG, FcγR antagonist gp34 recognizes further ligands involved in crucial defense mechanisms including
i) the B-cell receptor of IgG+ memory B-cells,
ii) C-reactive protein (CRP), playing important functions in complement activation but also host FcγR-dependent phagocytosis and
iii) the neonatal FcR (FcRn) potentially involved in the transplacental transmission of HCMV leading to congenital HCMV infection.

The potential of the sgp34wt and gp34W65F mAbs to interrupt the interaction of gp34 with IgGFc as well as other above-mentioned ligands was investigated. It is assumed that no immunomodulatory protein of microbial or pharmaceutical origin comparable to gp34 has been reported so far.

In addition, mAbs or binding fragments thereof according to the invention may be useful for highly sensitive and specific detection of the HCMV antigens gp34 and gp68 in clinical specimens as a diagnostic tool either in tissue sections or in immunoassays.

Taken together, the monoclonal antibodies or binding fragments thereof represent a novel, unique and promising approach to block immunoevasive functions of gp34 and gp68, thereby restoring immune effector functions of IgG and further ligands of gp34 including CRP, FcRn and IgG-BCR. Antibodies or antigen binding fragments specific for gp34 and gp68 can in general
1) improve the efficacy of HCMV-IgG,
2) improve B cell immunity in HCMV-infected patients,
3) prevent the transmission of HCMV by cellular transcytosis and
4) detect gp34 and gp68 in clinical specimens.

The monoclonal antibodies were raised against the IgG-Fc-binding ectodomains (ECDs) of the glycoproteins gp34 (encoded by the gene RL11) and gp68 (encoded by the gene UL119-118) of human Cytomegalovirus (HCMV). gp34 and gp68 are immune evasion molecules acting as viral Fcγ-Receptors (vFcγRs) antagonizing IgG-mediated immune responses of host FcγR-positive immune cells e.g. phagocytes and NK-cells.

gp34 and gp68 were identified as HCMV immune evasive molecules and further characterized. gp34 and gp68 both antagonize host FcγRI,-II and -III activation through interactions with the ligand IgG-Fc. Some of the mAbs according to the invention possess the potential to block this interaction between vFcγR gp34 or gp68 with IgG-Fc. By blocking vFcγR-IgG interaction, some of the mAbs described herein are able to restore IgG-mediated host FcγRs activation.

The following mAbs were generated:
8 mAbs raised against vFcγR gp68 with varying inhibitory potential to bind and block gp68-IgG binding.
9 mAbs raised against vFcγR gp34 wildtype (wt), with varying inhibitory potential to block IgG-gp34 binding; some mAbs that show no effect on IgG interaction however interfere with other gp34 ligands (vide infra).
6 mAbs raised against a point-mutation of gp34 (gp34mtrp). The mAbs recognize sgp34wt but differ in their inhibitory potential from mAbs raised against gp34 wt.

Surprisingly additional interaction partners of the viral glycoprotein gp34 were identified which includes binding to (i) the IgG-B-cell-receptor (BCR) on memory B-cells, (ii) C-reactive protein (CRP) and (iii) the neonatal Fc-Receptor (FcRn). The findings allow further uses of gp34 as a target to prevent HCMV dissemination and pathogenesis. No other protein of microbial or pharmaceutical origin seems to have comparable features as gp34.

Many experiments were performed and the results thereof are shown in the Figures. The following explanation of the Figures illustrates together with the graphs the present invention. In the present application the following abbreviations were used:
BCR B cell receptor
BSA bovine serum albumin
cCMV congenital cytomegalovirus
CDR complementary Determining Region
CFA complete Freund's adjuvant
CMV cytomegalovirus
CRP C-reactive protein
ECD ectodomain
ELISA Enzyme-linked immune-sorbent assay
Fab fragment antigen binding
Fc fragment crytallizable
FcRn neonatal Fc-receptor
Fcγ fragment crytallizable of immunoglobulin G subclass
FcγR Fcy (gamma)-receptor
FR frame work region
gp glycoprotein
HeLa human epithelial cells, cervix
hCMV human cytomegalovirus
HEK 293T Human embryonic kidney 293 cells
IC50 half maximal inhibitory concentration
ICOS-L Inducible T Cell co-stimulator ligand
IFA incomplete Freund's adjuvant
IgG immunoglobulin G
mAb monoclonal antibody
Moi multiplicity of infection
Mtrp mutated tryptophan
MW mother-well
SDS Sodium dodecyl sulfate
SP Signal peptide
TM transmembrane domain
VH variable heavy chain
VL variable light chain

To protect from antibody-mediated anti-viral immune responses herpesviruses evolved immunomodulatory molecules, so-called Fcγ-Receptors (vFcγRs), to counteract the host immune system.

IgG-dependent triggering of host FcγRs found on many immune cells leads to activation of phagocytic cells or antibody dependent cellular cytotoxicity (ADCC) by NK-cells. To antagonize IgG effector functions members of the herpesvirus family express their own, viral FcγRs to manipulate or compete with host FcγRs for ligand binding and thus interfere for example with IgG-mediated immune responses.

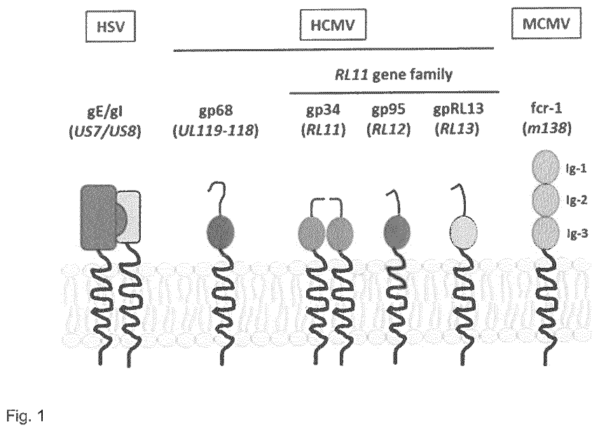

HCMV is equipped with several antagonists, i.e. gp34, gp68, gp95 and a fourth candidate, gpRL13 as shown schematically in FIG. 1. Those type-1 glycoproteins have in common to bind to the Fc-part of immunoglobulin G.

FIG. 1 shows herpesviral-encoded viral FcγRs. Several members of the herpesviral family express their own IgG-binding molecules to counteract and interfere with host FcγRs. Remarkably, in contrast to other members of this family, HCMV encodes not only one, but several of those antagonists: gp34 (RL11), gp68 (UL119-118), gp95 (RL12) and described only recently, gpRL13 (RL13). Here, gp34, gp95 and gpRL13 belonging to the RL11 gene family encoding several immune evasion molecules interfering with immune cells.

### Characterization of the Interaction of vFcγR gp34 with Human IgG

Viral glycoproteins gp34 and gp68 bind to the Fc-part of an IgG-molecule. For the experiments and the demonstration of their IgG-binding ability, recombinant proteins based on the HCMV AD169 strain were expressed by transient or stable transfection into HEK-293-T cells.

An ELISA-based assay was used to demonstrate binding of soluble, recombinant sgp34wt, a mutant thereof (gp34mtrp: containing one amino acid change from tryptophan to phenylalanine at position 65, leading to drastically impaired IgG-Fc-binding and inhibition), vFcγR gp68 and soluble ICOS-L as control protein (Inducible T Cell co-stimulator ligand for the T-cell-specific cell surface receptor ICOS).

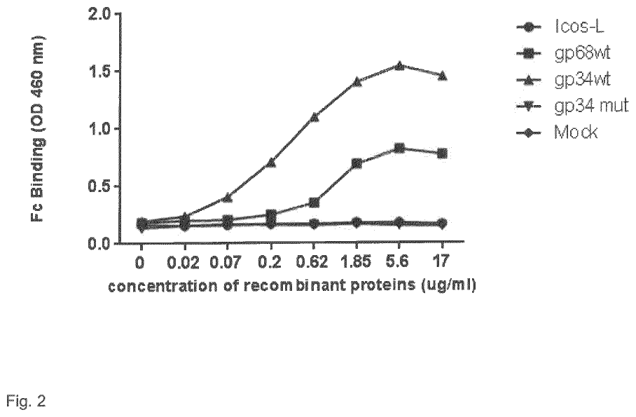

The binding to immobilized human IgG-Fc fragment by soluble, V5-His-tagged proteins was analyzed in ELISA in a dose-dependent manner. sgp34wt and gp68 are Fc-binding glycoproteins as shown in FIG. 2. A mutant version of sgp34wt, sgp34mtrp, lacks the ability for IgG-binding. In addition, the binding ELISA demonstrates ICOS-L to be a non-Fc-binding molecule serving as a suitable negative control during our assays.

FIG. 2: Dose-dependent IgG-Fc binding by vFcγRs gp34 and gp68. Binding to immobilized human IgG-Fc fragment by graded concentrations of recombinant vFcγRs sgp34wt and gp68 is demonstrated in ELISA. Hence, sgp34wt exhibits a stronger IgG-binding ability compared to sol gp68. The gp34 mtrp mutant shows no Fc binding as human ICOS-L and binding buffer (mock-treated control).

Viral Fc-binding molecules gp34 and gp68 interact and bind towards all human IgG-subclasses.

Surprisingly further ligands especially for glycoprotein gp34 were identified. Those interaction partners includes C-reactive protein (CRP), a key molecule of inflammation and part of the innate immune system as well as activator of the complement system, and the neonatal Fc-Receptor (FcRn).

C-reactive proteins share many structural but also functional features with antibodies, besides the obvious fact, to show binding towards Fc-Receptors. Also, since gp34 shows strong interference to the classical/canonical host FcγRs (FcγRI, FcγRIIa and -IIb, FcγRIII), this glycoprotein surprisingly interacts with the non-classical Fc-Receptor FcRn.

Beside manipulation(s) of IgG-mediated antiviral immune response, HCMV encodes for a molecule able to evade from CRP-mediated activation e.g. by phagocytes or by the complement system. On the other hand, interactions with the neonatal Fc-Receptor, involved in IgG half-life, transport (also placental transfer) and recycling represents new potential opportunities for the virus achieved by vFcγR gp34. This finding is the basis for new uses of the mAbs and binding fragments thereof according to the invention.

### Binding of vFcγR gp34 to C-Reactive Protein (CRP)

C-reactive protein (CRP) is a molecule of the innate immune system but has structural and functional features in common with antibodies (ref. Bang et al. 2005; Duncan et al. 1988). It has been reported that CRP binds to human FcγRs potentially leading to receptor activation. Since viral FcγRs like gp34 and gp68 were shown to bind to IgG, the binding potential of various HCMV vFcγRs towards the ligand CRP was investigated.

gp34, but neither gp68 nor gp95 were able to bind to immobilized CRP. This identifies a further host factor as an interaction partner of gp34 and provides evidence that the glycoproteins gp34 and gp68 differ in their features and functions. While gp34mtrp is strongly impaired regarding IgG-Fc-binding (see FIG. 2) it preserves binding capability to the CRP-ligand although at lower levels compared to the sgp34wt protein.

This suggests that different domains of gp34 are involved in IgG-versus CRP-binding. CRP-binding to gp34 was confirmed by pulldown experiments using western blot detection and flow cytometry as read-out system.

FIG. 3: Exclusive binding of C-reactive protein by vFcγR gp34. Various vFcγRs of HCMV were analyzed for their binding ability towards immobilized CRP in ELISA. While gp34 interacts with CRP, neither vFcγR sgp68, nor sgp95 bind to CRP. In addition, the IgG-Fc-deficient gp34 mutant sgp34mtrp shows residual binding to immobilized CRP.

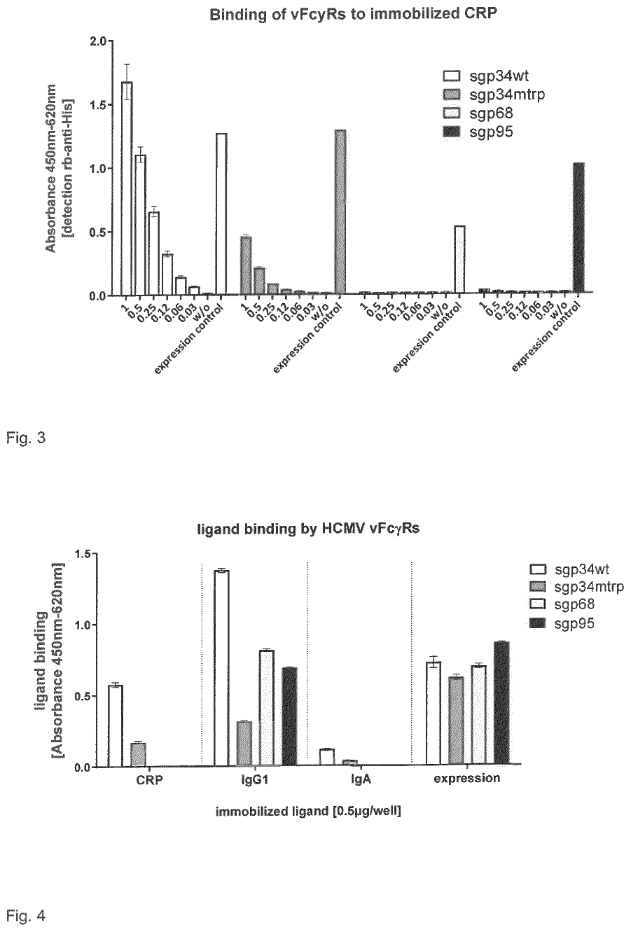

Specificity of vFcγR binding to CRP was approved by comparing the binding to various immunoglobulins including human IgG1 (positive control) and human IgA (negative control). The data confirmed specific binding by sgp34wt as well as the mutant sgp34mtrp (FIG. 4).

FIG. 4: In addition to IgG, also CRP is a ligand of vFcγR gp34. vFcγRs of HCMV were analyzed for their binding ability to immobilized CRP, human IgG1 or human IgA in ELISA. While gp34wt (also sgp34mtrp to some extent) was found to interact with CRP, neither vFcγR gp68, nor gp95 were found to bind to CRP. In contrast, all tested vFcγRs including gp34, gp68 and gp95 are truly IgG-Fc-binding proteins as demonstrated. Binding of vFcγR to immunoglobulins is restricted to the IgG subclass whereas there is no binding observed to the IgA subclass.

Next, it was investigated whether gp34 is able to antagonize CRP-mediated host FcγR activation, as was observed before for IgG-mediated FcγR-activation. To this end, a reporter-cell-based assay was modified measuring IgG-mediated FcγR-activation to detect CRP-mediated FcγR triggering. The assay is based on mouse BW5147 thymoma cells lacking T cell receptor chains which can be stably-transduced to express extracellular domains (ECDs) of various human FcγRs fused to the signaling module of the T-cell receptor (CD3-zeta chain). Upon FcγR crosslinking the reporter cells secrete mouse IL2 (mIL2). Therefore, mIL-2 measured by ELISA indicates the level of host FcγR-activation and quantifies antagonizing effects of vFcγRs.

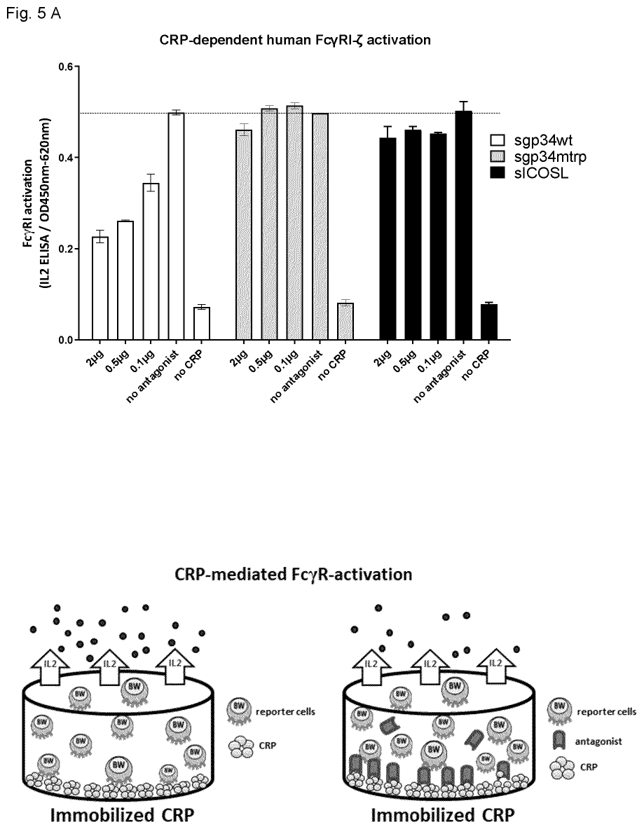

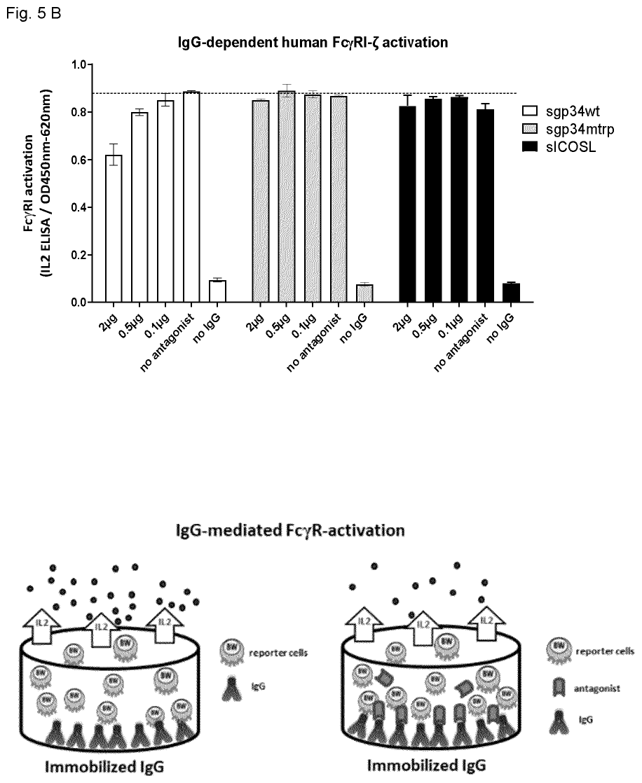

FIG. 5: Even stronger inhibitory effect by vFcγR gp34 on CRP-mediated host FcγRI activation, compared to IgG. Various recombinant, soluble proteins were used as potential antagonist to interfere with IgG- or CRP-dependent FcγR-activation using human FcγRI-expressing reporter cells. Our FcγR-activation assay was performed either on immobilized human IgG or on human CRP in presence or absence of vFcγR gp34 or control proteins. Gp34 showed an even stronger inhibitory effect on CRP-dependent FcγRI-activation compared to its antagonizing effect on IgG-dependent FcγRI-activation. sgp34mtrp or ICOSL ligand had no inhibitory effect on both ligands.

### Interaction of vFcγR gp34 with Neonatal FcR (FcRn)

The neonatal Fc-receptor (FcRn) is belonging the Fc-receptor family although it does not represent one of the classical, canonical human FcγRs FcγRI, -IIa, -IIb or -III. This molecule is mainly found in placenta, facilitating the transport of maternal IgG to the fetus. In addition, FcRn plays a role in regulating IgG and serum albumin half-life and turnover. FcRn-mediated transcytosis of IgG across epithelial cells is possible because FcRn binds IgG at acidic pH (<6.5) but not at neutral or higher pH. Therefore, FcRn can bind IgG from the slightly acidic intestinal lumen and ensure efficient, unidirectional transport to the basolateral side where the pH is neutral to slightly basic.

First, it was investigated whether the viral FcγR gp34 is capable to interact IgG-dependent or independent with neonatal Fc-Receptor. Using immobilized recombinant FcRn protein binding of gp34 and control proteins were investigated for dose-dependent binding ability in ELISA.

Our experiments identified FcRn, in addition to IgG, as a further ligand partner of vFcγR gp34, while control proteins like sgp34mtrp and ICOSL showed no binding.

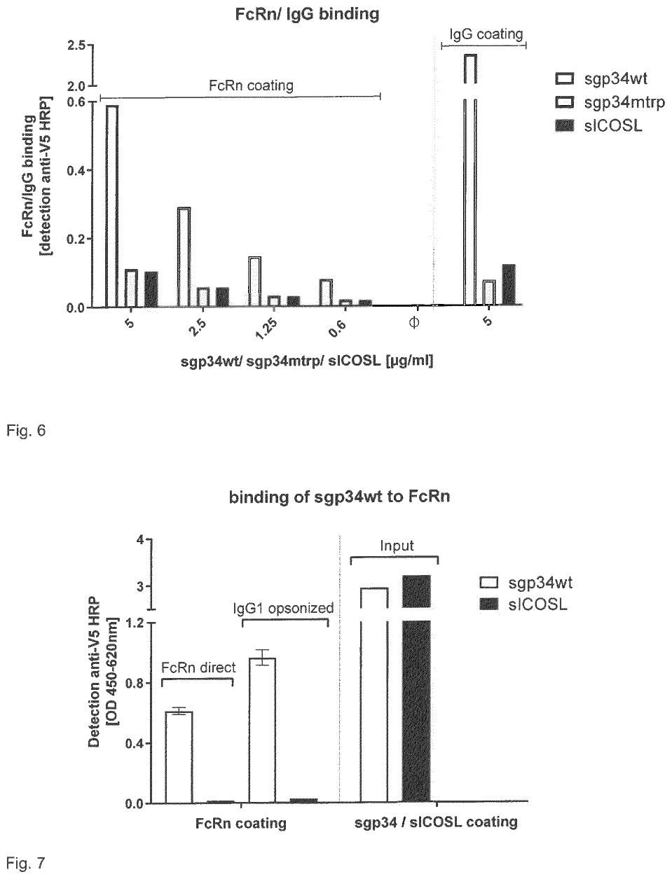

FIG. 6 shows that the neonatal Fc-receptor is bound by sgp34wt, but not sgp34mtrp. Binding ability of sgp34wt, sgp34mtrp and sICOSL as control protein where analyzed with regard to their ability to interact with FcRn. Recombinant FcRn was immobilized on ELISA plates and binding of sgp34wt, sgp34mtrp or sICOSL where detected via anti-V5-HRP antibody. Binding ELISA identified sgp34wt as a novel ligand of FcRn. In contrast, no binding of the mutant protein sgp34mtrp or sICOSL was observed, demonstrating specific interaction of sgp34wt with FcRn.

Next, it was investigated whether this binding is affected by the presence or absence of pre-bound IgG by FcRn. Indeed, ELISA binding studies showed enhanced binding of gp34 to IgG-opsonized FcRn. It is tempting to speculate if this is due to binding to the IgG-molecule itself or if pre-binding to IgG induces structural changes that further facilitate binding by gp34.

FIG. 7 demonstrates stronger binding of IgG-opsonized FcRn by gp34. Binding ability of sgp34wt and ICOSL as control protein where analyzed with regard to their ability to interact with FcRn opsonized with IgG or native FcRn. Recombinant FcRn was immobilized on ELISA plates and pre-incubated or not with human IgG. Binding of sgp34wt or ICOSL where detected via anti-V5-HRP antibody. Binding ELISA identified further enhanced FcRn binding by sgp34wt to FcRn decorated with human IgG. In contrast, no binding by ICOSL as control protein was observed, demonstrating specific interaction of sgp34wt with FcRn.

Next, interaction of sgp34wt and FcRn was analyzed in FACS experiments. Here, binding of gp34 to FcRn in absence or presence of antibodies was confirmed to be stable at neutral pH values as well as in low pH situations.

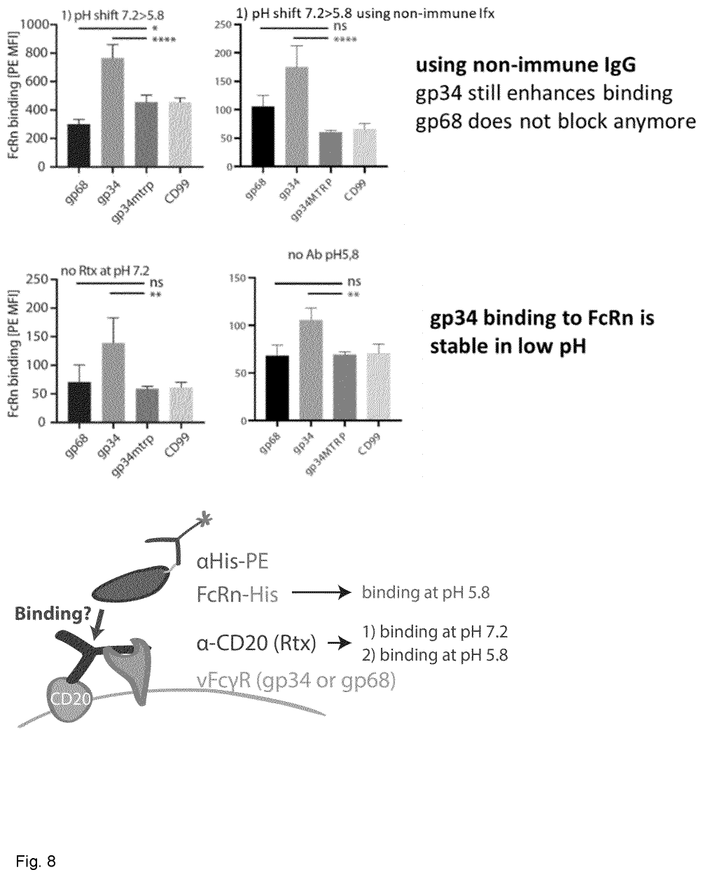

FIG. 8 shows that gp34 binding to FcRn is stable under low pH condition. Binding ability of sgp34wt, sgp34mtrp, sgp68 and sICOSL as control protein to FcRn where investigated by flow cytometry. The data revealed that vFcγR sgp34wt, but not gp34mtrp nor gp68 specifically interacts with FcRn. This interaction is observed on native, unbound FcRn, as well as in presence of antibodies. In addition, binding between sgp34wt and FcRn seems to be stable independent of the pH situation since FcRn still is bound by sgp34wt at low pH.

The observation that the HCMV-encoded Fc-binding protein gp34 shows now IgG-dependent but also direct binding towards FcRn led us to the speculation whether this interaction between gp34 and FcRn might have a role in transmission of HCMV from the mother to the fetus resulting in congenital infection.

Similar scenarios of viral transmission involving FcRn, with- or without IgG and viral particles have been shown recently. Studies demonstrated that FcRn-mediated transcytosis is involved with the trafficking of the HIV-1 virus across genital tract epithelium.

### Interaction of vFcγR gp34 to B-Cell Receptor (BCR)

The B-cell receptor (BCR) is a transmembrane protein on the surface of a B-cell. This receptor is composed of two molecules, CD79 and two immunoglobulin heavy and light chains. The membrane-bound immunoglobulin molecule of any given B-cell receptor is represented by one of the immunoglobulin isotypes: IgD, IgM, IgA, IgG, or IgE.

Experiments investigated binding of recombinant, soluble vFcγRs towards various immune cells to identify novel interaction partners/ligands. In brief, PBMCs were isolated from healthy donors, incubated with sgp34wt, sgp34mtrp, sgp68 or sICOSL. Next, immune cells were stained for characteristic surface markers and binding of soluble vFcγRs or sICOSL was determined using antibodies against the V5-His-tagged recombinant proteins.

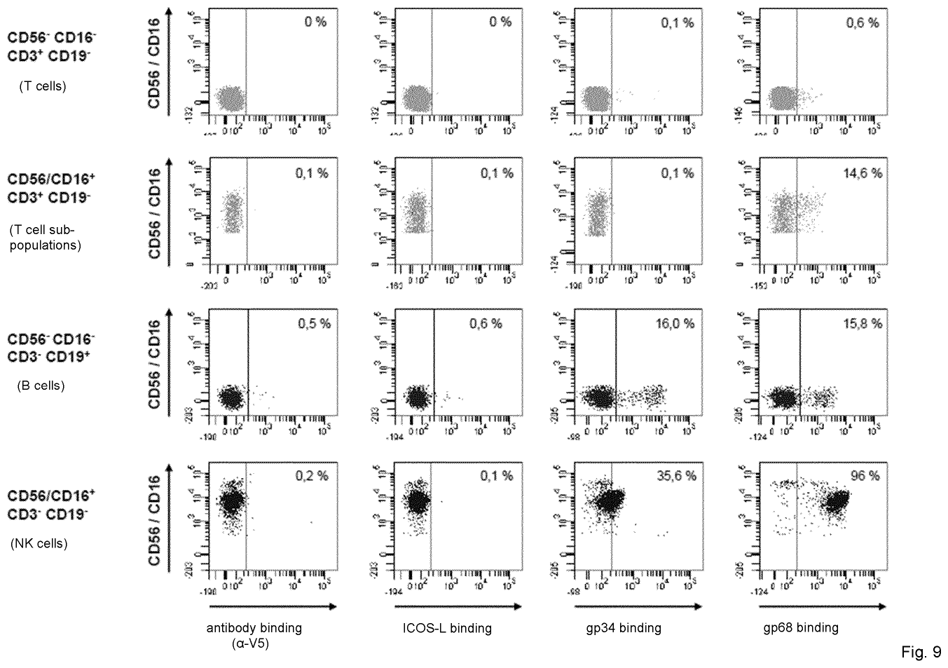

FIG. 9 shows the binding specificity of vFcγRs gp34 and gp68 to various subpopulations of immune cells. Recombinant, soluble proteins sgp34wt, sgp68 or sICOSL were incubated with various immune cell subpopulations. Binding analysis was done by flow cytometry using indicated surface markers for the identification of T-cells, B-cells or NK-cells. Binding of soluble proteins was determined by anti-V5-Tag as indicated. Both vFcγRs gp34 and gp68 show binding to a subpopulation of B-cells and NK-cells.

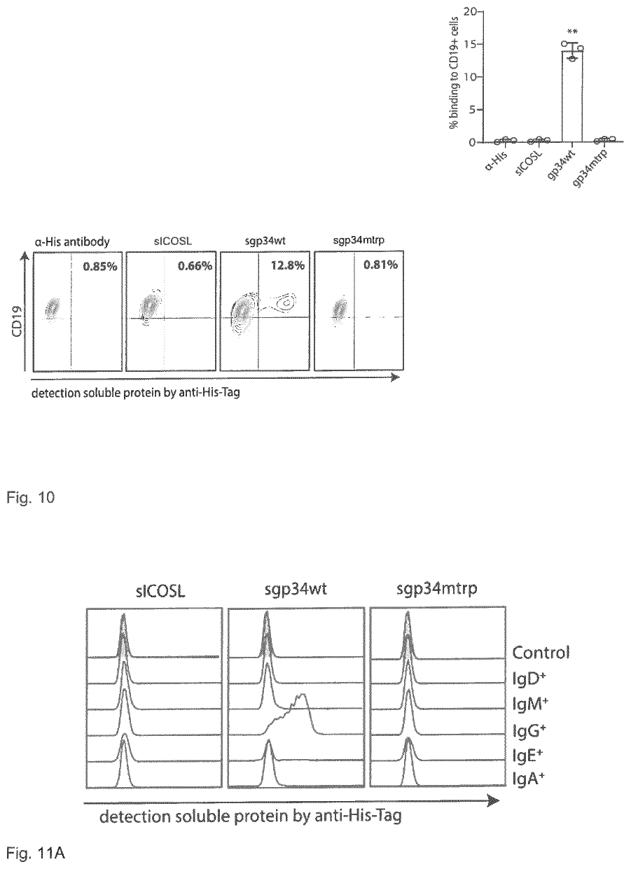

FIG. 10 describes binding specificity of vFcγRs gp34 to CD19+ B-cells. Recombinant, soluble proteins sgp34wt, sgp34mtrp or sICOSL were incubated with various immune cell subpopulations. Ex-vivo CD19+ human B cells were isolated from PBMCs via negative selection purification. Binding analysis was done by flow cytometry using indicated surface markers for the identification of T-cells, B-cells or NK-cells. Binding of soluble proteins was determined by anti-V5-Tag as indicated. Only vFcγRs gp34 shows binding to B-cells and NK-cells.

In a further experiment the immunoglobulin isotype of those CD19+ B-cells bound by gp34was investigated. Therefore, in addition, isotype-specific antibodies were used for co-staining in flow cytometry.

FIG. 11A shows the exclusive binding of vFcγRs gp34 to IgG+/CD19+ B-cells. Recombinant, soluble proteins sgp34wt, sgp34mtrp or sICOSL were incubated with various immune cell subpopulations. Binding analysis was done by flow cytometry using indicated surface markers for the identification of T-cells, B-cells or NK-cells. B cell isotype populations were distinguished using conjugated anti-IgG, -IgA, -IgM, -IgD or -IgE antibodies in flow cytometry. A positive double-staining with conjugated anti-immunoglobulin isotype and anti-His antibody indicated binding between B-cell isotypes and sgp34wt, sgp34mtrp or sICOSL. Flow cytometry analysis confirmed selective binding of IgG+/CD19+ B-cells by sgp34wt, not sgp34mtrp or sICOSL.

Finally, we used another approach to confirm specific interaction with sgp34wt and CD19+ B-cells of IgG-isotype. Here, an ELISA-based assay was conducted.

FIG. 11B illustrates the exclusive binding of vFcγRs gp34 to IgG+/CD19+ B-cells. Anti-IgG, -IgA, -IgM, -IgD or -IgE antibodies were coated onto an ELISA plate and incubated with ex-vivo isolated B-cells. sgp34wt, sgp34mtrp or sICOSL were co-incubated onto the ELISA plate. Recombinant proteins which bound indicated B-cell isotypes were detected with anti-His antibody. The optical density representing binding between proteins and B-cells was measured by spectrophotometry.

### Generation and Characterization of HCMV gp34 (RL11) and gp68 (UL119-118) Specific and Antagonizing Monoclonal Antibodies (mAbs)

### Recombinant Expression of Soluble vFcγRs sgp34wt, sgp34mtrp and sgp68 for Immunization

For generation of antigen specific and potential antagonizing monoclonal antibodies against vFcγR gp34 and gp68 of HCMV in total four different proteins where chosen to use as antigens for immunization. vFcγR gp34 and gp68 based on the sequence of HCMV AD169 strain, a non-functional mutant of gp34wt protein, gp34mtrp that will be further explained in detail later and ICOSL serving as control for antibody selection process containing the same protein-Tags (V5 and poly-His).

First, we have a closer look onto the recombinant protein of vFcγR gp34 and the point-mutation, gp34mtrp.

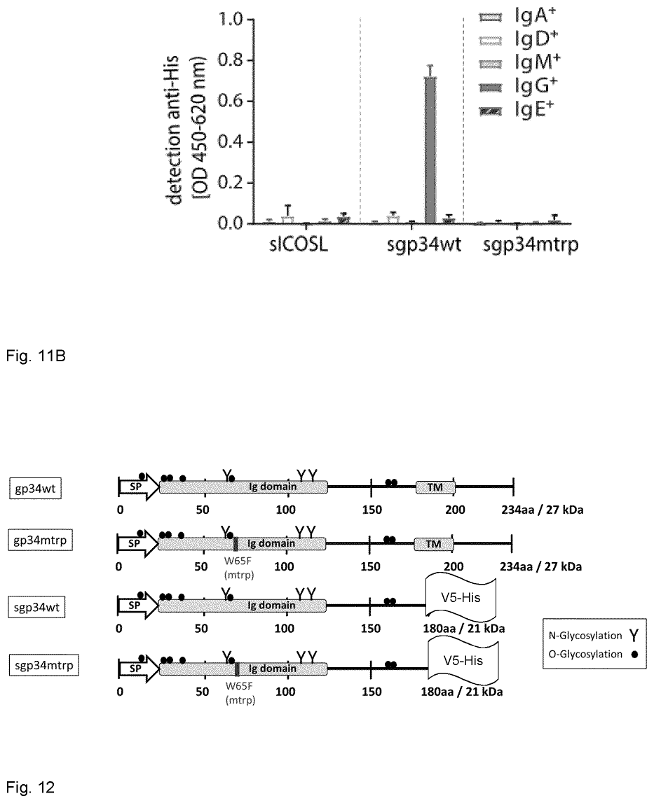

FIG. 12 shows schematically the domain structures of full length and soluble sgp34wt and gp34mtrp. Domain structure prediction based on protein sequences of HCMV AD169 vFcγR gp34 is depicted. Mutational analysis identified that a tryptophan on position 65 is important for oligomerization and impairs IgG-Fc-binding. This position (W65F, mtrp=mutated tryptophan) is highlighted in the gp34mtrp protein variants (highlighted in red letters). Full length versions of gp34wt and gp34mtrp are illustrated including the predicted signal peptide (SP), the Ig-like domain within the N-terminus, the transmembrane region (TM) followed by the cytoplasmic tail within the C-terminus. Glycosylation sites are predicted and illustrated: N-glycosylation (Y-shaped) and O-glycosylation (closed circle). In addition to full length versions, recombinant, soluble proteins are indicated, containing only the extracellular domain (ECD) and tagged with a V5-poly-histidine Tag. Soluble proteins are designated by the small letter “s” in front (sgp34wt and sgp34mtrp).

Next, we have a closer look onto the recombinant protein of vFcγR gp68.

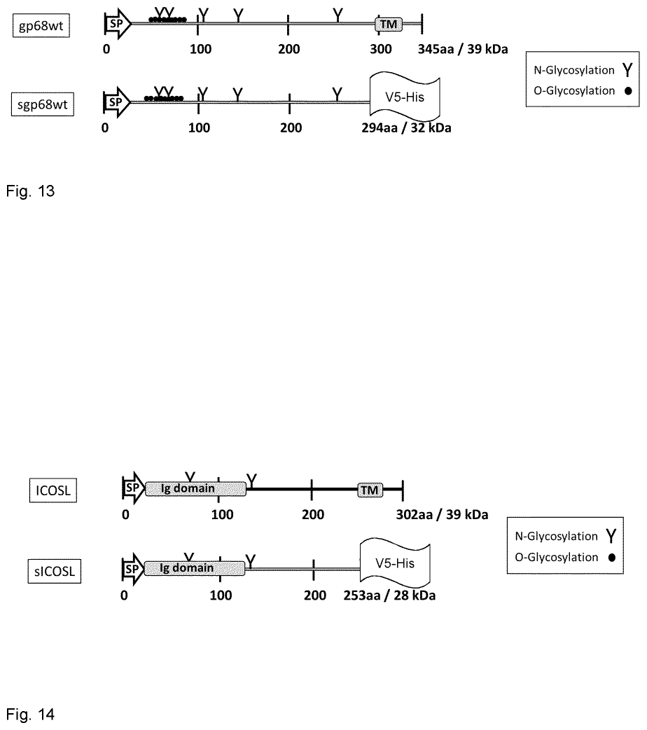

FIG. 13 shows domain structure of full length and soluble gp68. Domain structure prediction based on protein sequences of HCMV AD169 vFcγR gp68 is depicted. Full length version as well as a soluble variant of gp68 is illustrated including the predicted signal peptide (SP), the transmembrane region (TM) followed by the cytoplasmic tail within the C-terminus. Glycosylation sites are predicted and illustrated: N-glycosylation (Y-shaped) and O-glycosylation (closed circle). In contrast to the full-length gp68, the recombinant, soluble gp68 protein (sgp68) contains only the extracellular domain (ECD) and is V5-poly-His-tagged.

Finally, FIG. 14 illustrates the recombinant protein of human ICOSL, which was selected to serve as a negative control throughout experiments. Human ICOSL is also a type-I glycoprotein but without any known interactions to IgG.

The domain structure of full length and soluble human ICOSL is shown. Human ICOSL is a type-I glycoprotein, but without Fc-binding capacities. Domain structure prediction based on the protein sequence of human ICOSL (ICOS ligand). Full length version (ICOSL) as well as a soluble variant of ICOSL (sICOSL) is illustrated including the predicted signal peptide (SP), a predicted Ig-like domain, the transmembrane region (TM) followed by the cytoplasmic tail within the C-terminus. Glycosylation sites are indicated as following: N-glycosylation (Y-shaped) and O-glycosylation (closed circle). In contrast to the full-length ICOSL, the recombinant, soluble ICOSL protein (sICOSL) contains only the extracellular domain (ECD) and is tagged by V5-poly-histidine.

Only extracellular domains (ECDs) of the viral Fc-binding proteins gp34 and gp68 from HCMV AD169 strain were recombinantly expressed to serve as antigens for immunization of mice.

For expression, detection and purification purposes, soluble proteins were V5-His tagged on the C-terminal part of the ectodomains. N-terminus of gp34 was amplified by PCR using the following primers 5′-CGCGCTCGAGATGCAGACCTACAGCACCCC-3′ (SEQ ID NO:4) and 5′-CGCGGGATCCTCAATGGTGATGGTGATGATGACCGGTACGCGTAGAATCGAGACC GAGGAGAGGGTTAGGGATAGGCTTACCGGACCACTGGCGTTT-3′ (SEQ ID NO:5). Thereby, restriction sites were introduced, as well as the V5-His Tag using revere primer to add in-frame Tag onto the C-terminus before the Stop-codon. Cloning of gp34mtrp version was performed using same primers as for the construction of the sgp34wt plasmids, but using an already existing plasmid as PCR-template containing the amino acid change for the gp34mtrp version (W65F). Correct mutation was confirmed by DNA sequencing.

In the cloning of the N-terminus of gp68 suitable primers were used. The N-terminus of human ICOS ligand (ICOSL) was amplified by PCR using the following primers 5′-GCGCCTCGAGATGCGGCTGGGCAGTCCTGGACTG-3′ (SEQ ID NO:6) and 5′-GCGCGGATCCTCAATGGTGATGGTGATGATGACCGGTACGCGTAGAATCGAGACCGA GGAGAGGGTTAGGGATAGGCTTACCCGTGGCCGCGTT-3′ (SEQ ID NO:7). Again, C-terminal V5-poly-His Tag was added by revers primer. Sequencing of the coding sequences showed an amino acid exchange in ICOSL from V128 to I128 but with no detectable functional difference. Also, this V128 to I128 mutation is an described single-nucleotide polymorphism (SNP) of human ICOSL (NP_056074.1.1:p.Val128IIe).

All oligos used for amplification are listed in the table below.

TABLE 1
Overview oligos used for PCR amplification of soluble proteins sgp34wt, sgp34mtrp,
sgp68 and sICOSL. Restriction sites necessary for cloning, are indicated and highlighted in
italic. C-terminal, in-frame added V5-poly-histidin tag is double underlined. Restriction sites as
well as C-terminal tag were added in-frame using respective reverse primer.

| | | Restriction | |
|---|---|---|---|
| Name | Sequence 5′ → 3′ | site | C-terminal Tag |
| sgp34_Xhol_for | CGCGCTCGAGATGCAGACCTACAGCA | 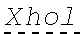 | — |
| | CCCC (SEQ ID NO: 8) | | |
| sgp34_BamHI_rev | 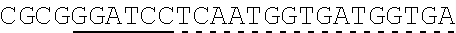 | 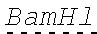 | V5-His |
| | 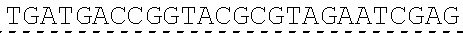 | | |
| | 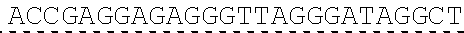 | | |
| | 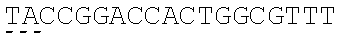 | | |
| | (SEQ ID NO: 9) | | |
| sgp68_Xhol_for | CGCGCTCGAGATGTGTTCCGT | 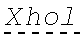 | — |
| | (SEQ ID NO: 10) | | |
| sgp68_BamHI_rev | 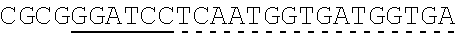 | 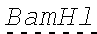 | V5-His |
| | 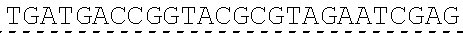 | | |
| | 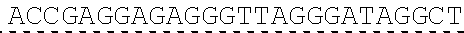 | | |
| | 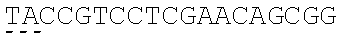 | | |
| | (SEQ ID NO: 11) | | |
| sICOSL_Xhol_for | GCGCCTCGAGATGCGGCTGGGCAGT | 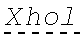 | — |
| | CCTGGACTG (SEQ ID NO: 12) | | |
| sICOSL_BamHI_rev | 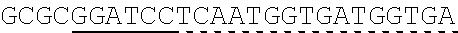 | 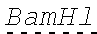 | V5-His |
| | 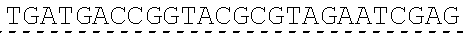 | | |
| | 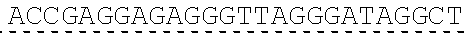 | | |
| | 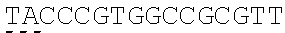 (SEQ ID NO: 13) | | |

Briefly, each PCR product was amplified and cloning restriction sites were inserted by the forward and the revers primers. Both protein Tags (V5 and poly-histidine) were inserted in-frame using the revers primer. PCR-products were purified from agarose-gel using Monarch gel purification kit. Inserts and target vectors were digested for overnight and gel-purified again. Cloning was done, using the restriction sites XhoI and BamHI (italics in primer sequences) and then cloned into pIRES-EGFP vector (Clontech, USA). For enhanced protein expression, ß-Globin cloned from pSG5 vector (Stratagene, USA) was inserted into pIRES-EGFP between the CMV-IE promoter and the coding sequences, named pIRE-ß-EGFP. Inserts coding for tagged sgp34wt, sgp34mtrp, sgp68 and sICOSL were further sub-cloned into pUCIP plasmid using same restrictions sites as before to use them for lentiviral transduction of target cells like HEK-293-T.

Recombinant protein expression was conducted by two different expression strategies, both containing their own advantages and disadvantages. Proteins were expressed either (i) by transiently transfection of HEK-293T cells or (ii) by stable HEK-293T cells after lentiviral transduction using pUCIP plasmids and puromycin selection of stable clones after sub-cloning by limiting dilution technology. The plasmids were transfected in HEK-293-T cells using polyethylemine (PEI, branched, Sigma-Aldrich, Germany) and transfected cells were verified by expression of EGFP (expressed also on the pIRES-ß-EGFP plasmid) by fluorescent microscopy (Sigma-Aldrich, Germany).

For protein expression in stable cells three human cell lines where tested for best quality and quantity. Therefore, Hela cells, HEK-293-T and A549 cells where stably transduced by lentiviral transduction as described elsewhere and protein expression was compared and analyzed. On basis of those results, HEK-293-T cells where selected for further recombinant protein expression of soluble proteins to use for immunization.

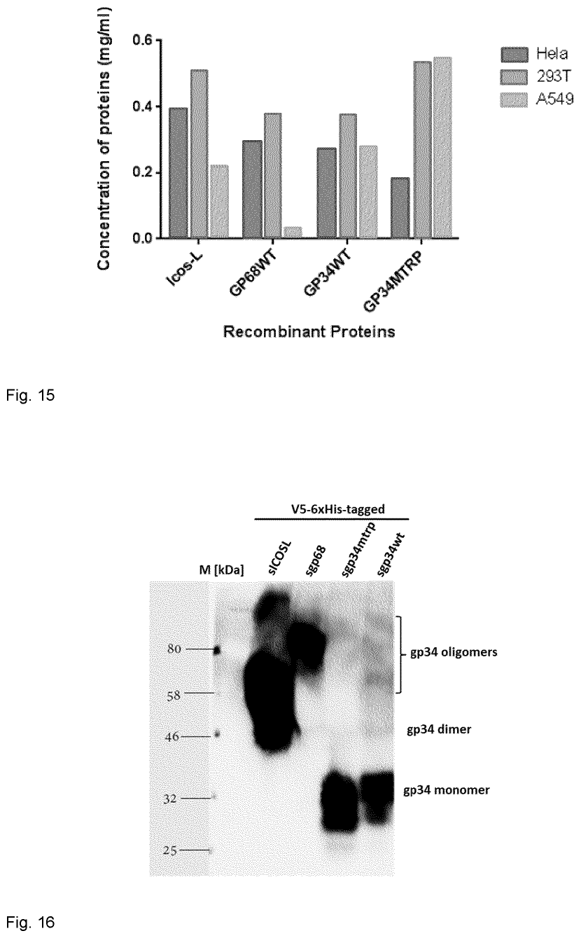

FIG. 15 shows a representative comparison of protein expression in various stable cell lines. Various cells were lentivirally transduced for stable expression of soluble proteins (sgp34wt, sgp34mtrp, sgp68 and sICOSL). HeLa (human epithelial cells, cervix, blue bars), HEK-293-T (human epithelial cells, embryonic kidney, green bars) and A549 (human epithelial cells, lung epithelium, orange bars), cells were chosen as target cells and cells were controlled for successful protein expression after puromycin selection. Expression levels of secreted proteins within the cell culture supernatant were analyzed by rabbit-anti 6×His antibody in ELISA. Stable 293-T cells were used for further purposes, showing most convincing protein expression for all proteins.

Since earlier experiments demonstrated problems during purification process and affected recombinant protein quality, we decided to produce our recombinant without fetal calf serum (FCS) to avoid contamination with proteins like bovine serum albumin (BSA).

When cells were 90-100% confluent and well-attached media was carefully replaced by starvation medium without FCS (DMEM w/o phenol red, 1% Pen/Strep, 1% Sodium Pyruvat). After 5-7 days or when cells started to detach, supernatants were collected, remaining cells in the supernatant removed by centrifugation (40 min, 4.000 g), sterile filtered and adjusted to a 10 mM Imidazole concentration and passed over a His-Trap FF crude column (GE Healthcare, USA). Proteins were eluted in Imidazole/Phosphate buffer (250 mM Imidazole, 20 mM sodium phosphate, 500 mM NaCl) and immediately dialyzed against PBS and concentrated via Amicon columns. Protein concentrations were determined by PIERCE BCA assay and protein quality was analyzed in various assays like IgG-Fc binding ELISA, coomassie stain and western blot using anti-V5 or anti-His-Tag antibodies for detection. For second expression strategy, using stable cell clones for expression of recombinant proteins, cells were kept under selection pressure with 2 mg/ml puromycin (selection marker encoded on the pUCIP plasmids). Cells were grown and expanded to a density of ˜100% per dish and put under Starvation as described before. Supernatants were collected usually between 7-10 days when cells started to detach from the surface. Further steps including collection, purification and dialysis was performed as mentioned before.

FIG. 16 shows the expression of soluble, V5-poly-His tagged glycoproteins. Purified, soluble glycoproteins sgp34wt, sgp34mtrp, sgp68 and sICOSL were characterized in western blot. Proteins expressed from stable 293-T cells were purified dependent on their V5-6×His-Tag by NiNTA purification using ÄKTA system, dialyzed and concentrated against PBS. 5 μg total protein was loaded on SDS-PAGE and proteins detected in rabbit-anti-His western blot.

Protein expression and purification was further optimized to obtain proteins best in quality and quantity. Next, a scheme for a typical strategy for recombinant protein expression, supernatant collection and protein purification is illustrated.

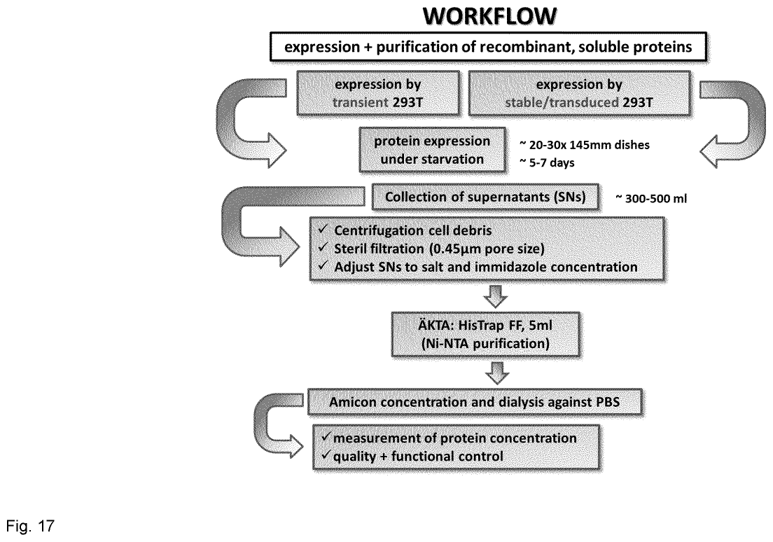

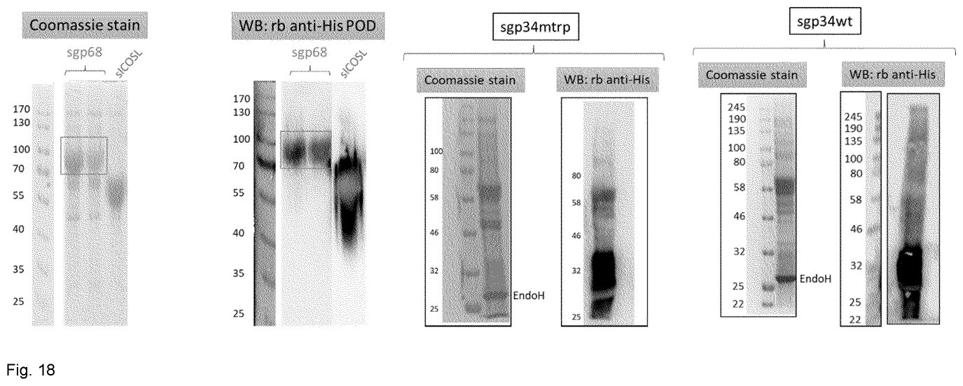

FIG. 17 is a workflow of recombinant protein expression and purification. Essential Steps of recombinant protein expression and purification is illustrated. Quality and quantity controlled proteins were used for immunization process.

### Antigens Used for Immunization

Protein batches were generated, quality controlled and protein concentrations measured using PIERCE BCA kit. Selected samples were delivered on dry ice to our collaboration partners to performing immunization, fusion and sub-cloning of promising candidate clones. Protein batches used for immunization are illustrated following.

FIG. 18 shows the recombinant proteins used as antigens for immunization. Purified, soluble ectodomains of sgp34wt, sgp34mtrp, sgp68 and sICOSL were characterized in coomassie stain and western blot. 10 μg of purified proteins expressed from stable 293-T cells were loaded on SDS-page for coomassie stain. In parallel 10 μg of proteins were loaded for western blot analysis using rabbit-anti-His and goat-anti-rabbit IgG HRP for detection.

### Antibody Selection Process for gp34wt, gp34mtrp and qp68

The immunogen (sgp68, sgp34wt or sgp34mtrp) expression technology is based on recombinant protein production in HEK-293-T cells. In contrast to wildtype gp34, the mtrp version of gp34wt protein consists of a single amino acid change within the extracellular domain. The original tryptophan (Trp) at position 65 was mutated to phenylalanine (Phe) which leads to conformational consequences in the protein.

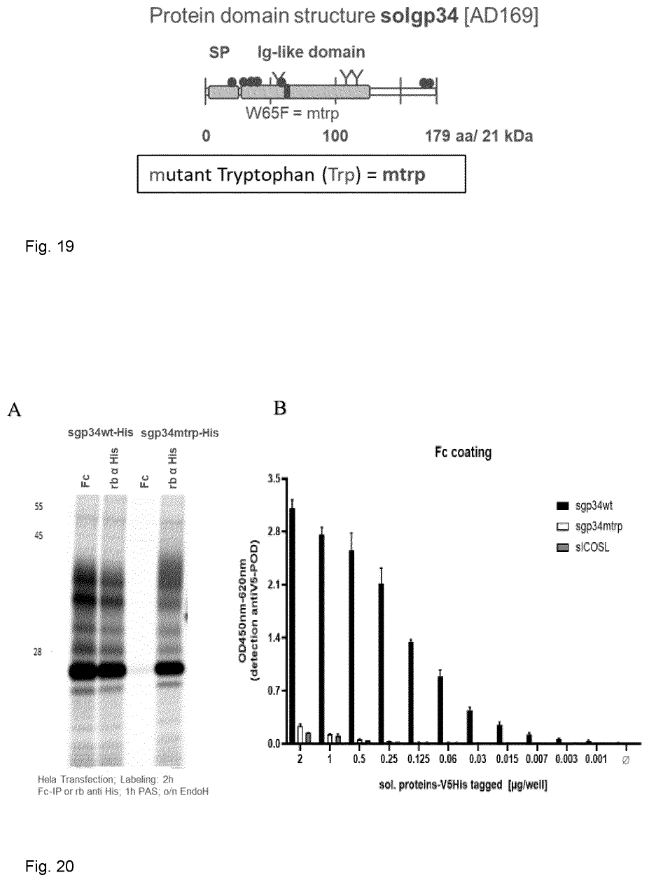

FIG. 19 emphasizes the tryptophan in gp34wt sequence which is essential for its function. sgp34wt and sgp34mtrp proteins were used for immunization of mice to enhance the chance of successful antibody generation. The tryptophan (trp) position within the extracellular domain (W65), important for IgG-Fc-binding is indicated.

Usually gp34wt protein forms monomers, dimers and high molecular weight oligomers like tetramers. However, oligomerization seems to be essential for IgG interaction. In consequence, gp34mtrp forms only monomers and dimers and lacks IgG-Fc binding capacity as shown in FIG. 20.

Next, gp34wt and gp34mtrp variants were further characterized, regarding their ability to bind human IgG-Fc-fragment. The data demonstrate that gp34 is indeed a vFcγR able to bind to human IgG shown in precipitation experiments and binding ELISA. Both experiments show as well that gp34 with a mutated tryptophan (gp34mtrp) is impaired for IgG-Fc-binding.

FIG. 20: mutated tryptophan in gp34wt sequence is essential for IgG binding. A mutation at position 65 on amino acids level results in a Fc-binding impaired gp34 variant-gp34mtrp. Lack of IgG-binding by this point mutation is shown using precipitation of radioactive-labeled proteins (A) or via binding ELISA on immobilized human IgG-Fc-fragment (B). Both molecules in their soluble, V5-His-tagged form were expressed in transiently transfected Hela cells. 1 day post transfection, cells were metabolically labeled using ³⁵S for 2 hours (see FIG. 20A). Cell lysates were incubated with indicated antibodies either precipitating the soluble proteins, sgp34wt or sgp34mtrp, via C-terminal His-Tag or using human IgG-Fc-fragment to precipitate protein capable for Fc-binding. Precipitation of EndoH-deglycosylated samples was analyzed using SDS-gradient gel and Phospholmage software for development. Results confirmed comparable expression of both molecules, but drastically impaired Fc-binding for sgp34mtrp. Impaired IgG-Fc-binding was confirmed in binding ELISA using immobilized human Fc-fragment (see FIG. 20B). Brief, human Fc-fragment (Rockland; at 4 μg/ml) was immobilized on ELISA plates. Soluble proteins were added to blocked wells and binding detected using anti-His antibody recognizing C-terminal tagged sgp34wt, sgp34mtrp or sICOSL, used as a non-binding control.

Since gp34wt protein shows in addition to human IgG, also binding potential to various mouse IgG subclasses, we decided to use also gp34mtrp for immunization to guarantee a successful generation of anti-gp34 mAbs. Cells were transfected for expression of sgp34wt and sgp34mtrp proteins and analyzed for their binding to various IgG molecules including human IgG and various mouse subclasses. As expected, sgp34mtrp is not only impaired for binding to human, but also to mouse IgG molecules and should therefore not interfere with the immunization process.

It was questionable in the beginning of this process whether it was possible to generate mAbs using sgp34wt protein as antigen that would truly recognize the vFcγR gp34 or if such antibodies would only be “false-positive” due to binding by gp34wt to these antibodies via their Fc-part. Therefore, it was speculated to gain at least antibodies using the gp34mtrp variant, structurally forming monomers and dimers, also recognizing the native protein.

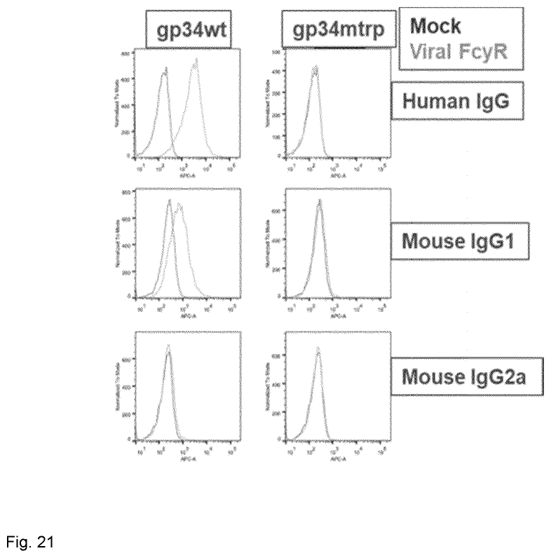

FIG. 21 illustrates the binding of vFcγR gp34 to mouse IgG subclasses. While sgp34wt protein shows binding to the Fc-part of human IgG and at least mouse IgG1, gp34mtrp protein is impaired for IgG binding in general, independent on the origin of IgG-species used.

### Immunization of Female BALB/c Mice

The sequences of gp34wt and gp68wt correspond to the laboratory HCMV strain AD169. Nethertheless, both proteins are highly conserved between HCMV strains, showing only few amino acid variations.

For immunization, BALB/c mice were injected with 50 μg of the immunogen (either sgp68-V5-His, sgp34wt-V5-His or sgp34mtrp-V5-His) in complete Freund's adjuvant (CFA) followed by 50 μg of the same immunogen in incomplete Freund's adjuvant (IFA) at day 14 post first immunization. Next, ELISA analyzes of the sera for anti-gp68, anti-sgp34wt or anti-gp34mtrp antibody titers were performed. The mice with the highest titer were boosted with another 50 μg of the respective immunogen in PBS.

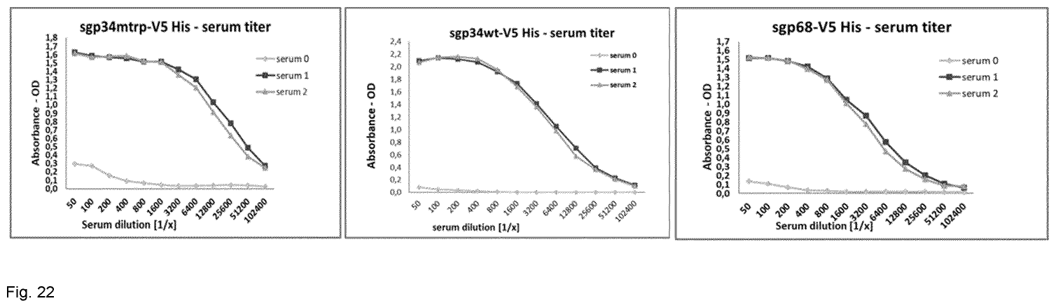

FIG. 22: immunization titers of BALB/c mice immunized with sgp34mtrp, sgp34wt or sgp68. Serum titers for sgp34mtrp, sgp34wt and sgp68 immunization are shown. Sera tested at different intervals for antigen-specificity in ELISA. Dependent on serum-titers compared to non-immunized individuals, mice were killed and splenocytes used for fusion.

After three days, the spleen of immunized mice was taken and, after lysis of red blood cells, the splenocytes were fused with SP2/0 cell line. The potential hybridoma cells were seeded in 20% RPMI 1640 medium containing hypoxanthine, aminopterin, and thymidine (HAT) for selection of stable hybridoma cell lines.

To obtain anti-gp68 antibodies, 564 wells with hybridoma cells were screened for antibody secretion by ELISA. Since all proteins gp68, sgp34wt, sgp34mtrp and sICOSL were C-terminal V5 and poly-His tagged one has to test and exclude those clones only raised against the protein-Tag itself.

Then, 59 hybridoma cells from wells in which the supernatants were positive on the gp68-V5His coated ELISA plates, were re-tested for their positivity. In parallel, a cross-reactivity test was performed on an irrelevant His-tagged protein. This resulted in 17 hybridoma cells that secreted antibodies specifically recognizing gp68 protein. Moreover, none of these antibodies showed cross-reactivity to the sgp34mtrp protein (data not shown) or the irrelevant control protein (sICOSL-V5His).

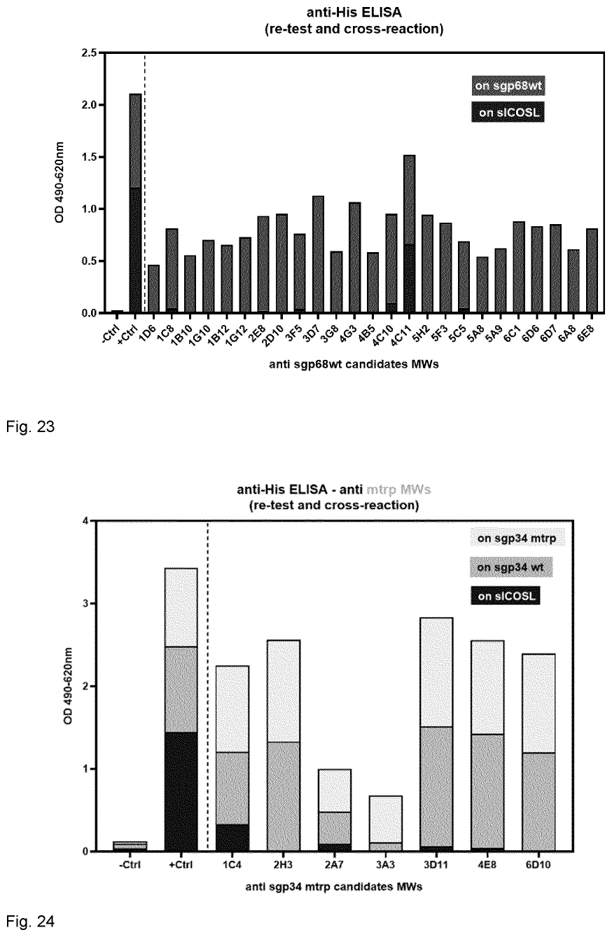

FIG. 23: First screening candidates anti-vFcγR gp68 mAbs. Supernatants positive after first round of screening were re-tested for antigenic specificity and tested on control protein to exclude antibodies raised against V5-His-Tag only. Supernatants of cell cultures of various hybridomas were analyzed on immobilized sgp68 (V5-His tagged) or control protein (sICOSL, also V5-His-tagged). ELISA values for detection of sgp68 (red part of the bars) or sICOSL (blue part of the bars) are shown additive. Clone names are indicated as origin of mother well (MW) of mAb candidates.

Out of 17 hybridoma cells, 4 were further selected due to their capacity to block the binding of the human Fc-fragment to the gp68 protein and sub-cloned. At this point, 8 stable clonal cell lines, summarized in Tab. 2, were selected for large scale production and purification (gp68.01, gp68.02, gp68.03, gp68.04, gp68.05, gp68.06, gp68.07, gp68.08). Thereby, always to pairs originated from one mother-well (MW) and may most likely result in identical sub-clones.

TABLE 2
Overview of potential anti-vFcγR gp68 mAbs candidates. 4 clones
were chosen out of positive clones after first ELISA screenings.
Clones were further sub-cloned by limiting dilution to obtain
monoclonal antibodies. Original mother-wells (MW) are indicated
within the table and also indicate clones potentially derived
from the same hybridoma. In addition, subclass specificity was
determined by ELISA. Only 2 candidates belong to the mouse IgG2a
subclass, whereas the others are mouse IgG1.

| | MW | Subclass |
|---|---|---|
| gp68.01 | 3D7 | mIgG1 |
| gp68.02 | 3D7 | mIgG1 |
| gp68.03 | 5H2 | mIgG1 |
| gp68.04 | 5H2 | mIgG1 |
| gp68.05 | 6D7 | mIgG1 |
| gp68.06 | 6D7 | mIgG1 |
| gp68.07 | 4C10 | mIgG2a |
| gp68.08 | 4C10 | mIgG2a |

Next, a large-scale antibody production was performed and all monoclonal antibodies were purified from the serum free medium, using GE ÄKTA Prime Plus Liquid Chromatography System and HiTrap Protein G columns, in an amount of few milligrams. Finally, all 8 leading candidates showed a strong binding capacity to the purified protein but moreover to the native HCMV gp68 protein in infected cells.

Next, to obtain anti-gp34wt antibodies, 564 wells with hybridoma cells were screened for antibody secretion by ELISA. Then, 34 hybridoma cells from wells in which the supernatants were positive on the sgp34wt coated ELISA plates, were re-tested for their positivity. In parallel, a cross-reactivity test was performed on an irrelevant V5-His-tagged protein (e.g. sICOLS-V5-His). This resulted in 14 hybridoma cells that secreted antibodies specifically recognizing sgp34wt protein. Moreover, none of these antibodies showed cross-reactivity to the sgp68 protein (data not shown). Interestingly, no antibody obtained using sgp34wt protein for immunization recognized the sgp34mtrp protein having only one amino acid altered. This tempted us to speculate that gp34wt mAbs recognize structural epitopes on gp34 since its mtrp version is impeded for oligomerization.

Out of 14 hybridoma cell lines, 5 were further selected for their highest absorbance and sub-cloned. At this point, 9 stable clonal cell lines, were selected for large scale production and purification (gp34wt.07, gp34wt.08, gp34wt.09, gp34wt.10, gp34wt.11, gp34wt.12, gp34wt.13, gp34wt. 14, gp34wt.15). Thereby, always two pairs originated from one mother-well (MW) and may result in identical sub-clones. mAb gp34wt.15 is unpaired.

TABLE 3
Overview potential candidates anti-vFcγR gp34wt mAbs. 5 clones
were chosen out of positive clones after first ELISA screenings.
Clones were sub-cloned by limiting dilution to obtain monoclonal
antibodies. Original mother-wells (MW) are indicated within the
table and also indicate clones most likely derived from the same
hybridoma. In general, 2 sub-clones from 1 original mother-well
(MW) were further characterized. In case of MW 3F5 only one sub-
clone stayed positive. In addition, subclass specificity was determined
by ELISA and is indicated. Only 2 candidates belong to the mouse
IgG2a subclass, whereas the others are mouse IgG1.

| | MW | subclass |
|---|---|---|
| gp34wt.07 | 4B4 | IgG1 |
| gp34wt.08 | 4B4 | IgG1 |
| gp34wt.09 | 4H7 | IgG1 |
| gp34wt.10 | 4H7 | IgG1 |
| gp34wt.11 | 5A11 | IgG1 |
| gp34wt.12 | 5A11 | IgG1 |
| gp34wt.13 | 6E3 | IgG2a |
| gp34wt.14 | 6E3 | IgG2a |
| gp34wt.15 | 3F5 | IgG1 |

Next, a large-scale antibody production was performed and all monoclonal antibodies were purified from the serum free medium, using GE ÄKTA Prime Plus Liquid Chromatography System and HiTrap Protein G columns, in an amount of few milligrams. Finally, all 9 leading candidates showed a strong binding capacity to the native HCMV gp34 protein in infected cells.

Finally, to obtain anti-gp34mtrp antibodies, 1128 wells with hybridoma cell were screened for antibody secretion by ELISA. Then, 12 hybridoma cells from wells in which the supernatants were positive on the sgp34mtrp coated ELISA plates, were re-tested for their positivity. In parallel, a cross-reactivity test was performed on an irrelevant His-tagged protein (sICOSL-V5His). This resulted in 5 hybridoma cell lines that secreted antibodies specifically recognizing sgp34mtrp protein. Moreover, all 5 antibodies showed cross-reactivity to the sgp34wt protein.

Out of 5 hybridoma cell lines, 3 were further selected for their highest absorbance and sub-cloned. At this point, 6 stable clonal cell lines, were selected for large scale production and purification (gp34mtrp.01, gp34mtrp.02, gp34mtrp.03, gp34mtrp.04, gp34mtrp.05, gp34mtrp.06). Next, a large-scale antibody production was set up and all monoclonal antibodies were purified from the serum free medium, using GE ÄKTA Prime Plus Liquid Chromatography System and HiTrap Protein G columns, in an amount of few milligrams. Finally, all 6 leading candidates showed a strong binding capacity to the native HCMV gp34 protein in infected cells.

FIG. 24: First screening anti-vFcγR gp34 mAbs candidates, derived from immunization using sgp34mtrp as immunogen. Supernatants positive after first round of screening were re-tested for antigenic specificity and tested on control protein to exclude antibodies raised against V5-His-Tag only and tested for recognition of sgp34mtrp as well as sgp34wt protein. Supernatants of cell cultures of various hybridomas were analyzed on immobilized sgp34wt (V5-His tagged), sgp34mtrp (V5-His tagged) or control protein (sICOSL, also V5-His-tagged). ELISA values for detection of sgp34wt (green bars), sgp34mtrp (yellow bars) or sICOSL (blue bars) are shown additive. Clone names are indicated as origin of mother-well (MW) of mAb candidates.

Although not many positive candidates derived from the immunization were obtained using sgp34mtrp protein as antigen, nearly all of them recognize gp34mtrp as good as they recognize native gp34 protein.

TABLE 4
Overview anti-vFcγR gp34 mAbs candidates, using sgp34mtrp
as antigen. Illustrated hybridoma clones were chosen out of
positive clones after first ELISA screenings. Clones were
sub-cloned by limiting dilution to obtain monoclonal antibodies.
Original mother-wells (MW) are indicated within the table
and also indicate clones potential deriving from the same
hybridoma. In general 2 sub-clones from 1 original mother-
well (MW) was further characterized. In addition, subclass
specificity was determined by ELISA and is indicated. All
potential mAbs candidates belong to the mouse IgG1 subclass.

| | MW | subclass |
|---|---|---|
| mtrp.01 | 2H3 | mIgG1 |
| mtrp.02 | 2H3 | mIgG1 |
| mtrp.03 | 3D11 | mIgG1 |
| mtrp.04 | 3D11 | mIgG1 |
| mtrp.05 | 4E8 | mIgG1 |
| mtrp.06 | 4E8 | mIgG1 |

In summary, we generated various monoclonal antibodies recognizing vFcγRs gp34 or gp68 that were further characterized and functional analyzed. All candidates, including the mouse IgG subclass belonging to, are summarized in following table (Tab. 5).

TABLE 5
Overview potential candidates of anti-vFcγR gp34
and anti-gp68 mAbs. Various sub-clones recognizing
gp34wt, gp34mtrp or gp68 protein are depicted. Mouse
IgG-subclasses are indicated in brackets ( ).

| α gp34 mtrp (AD169) | α gp34 wt (AD169) | α gp68 (AD169) |
|---|---|---|
| mtrp.01 (mIgG1) | gp34wt.07 (mIgG1) | gp68.01 (mIgG1) |
| mtrp.02 (mIgG1) | gp34wt.08 (mIgG1) | gp68.02 (mIgG1) |
| mtrp.03 (mIgG1) | gp34wt.09 (mIgG1) | gp68.03 (mIgG1) |
| mtrp.04 (mIgG1) | gp34wt.10 (mIgG1) | gp68.04 (mIgG1) |
| mtrp.05 (mIgG1) | gp34wt.11 (mIgG1) | gp68.05 (mIgG1) |
| mtrp.06 (mIgG1) | gp34wt.12 (mIgG1) | gp68.06 (mIgG1) |
| | gp34wt.13 (IgG2a) | gp68.07 (IgG2a) |
| | gp34wt.14 (IgG2a) | gp68.08 (IgG2a) |
| | gp34wt.15 (mIgG1) | |

### Maintenance and Expansion of Hybridomas

Hybridomas were tested in ELISA for specific antibody production. Positive clones were sub-cloned and further tested. When clones remained positive in ELISA, they were cultured and transferred from 96-well plates to larger vessels and later to flasks/dishes. Cells were expanded in RPMI, 10% FCS media to desired cell density. For antibody production and collection, the FCS percentage in the culture medium was reduced from 10% to 0% (Starvation media contains no FCS at all). For starvation, cells were re-suspended after centrifugation into ISF-1 medium (F 9061-01 from Biochrom) without any additional supplements. Cells were routinely checked for viability under microscopy. After 2-3 weeks, the conditioned medium was collected by centrifugation (4.000 g, 40 mins, 4° C.) and sterile-filtered (0.45 μm in size] into flasks until the antibody purification on protein G sepharose (use HiTrap® Protein G HP GE Healthcare 1 ml columns) was down.

### Freezing Hybridomas

Hybridoma cells and corresponding sub-clones were frozen for long-time storage in liquid nitrogen. For this, first the cell number was determined by counting them with trypan-blue staining. Then, 0.5-1×107 cells/ml were re-suspended in fresh medium (RPMI medium with 10% FCS) and diluted 1:2 in 2× freezing medium. Sample preparation was performed on ice. Samples were stored on liquid nitrogen.

### Purification of Monoclonal Antibodies by Protein-G Sepharose

In general, antibodies were purified using 1 ml protein-G sepharose, packed into a column and attached to the GE Healthcare ÄKTA System device. Before loading the column with the antibody solution, the resin was pre-equilibrated. A four-fold volume of binding buffer was passed through the column with a flow rate of 0.5 ml/min. Then the antibody solution was loaded onto the protein G sepharose column at the same flow rate. Non-specifically bound proteins were washed off with binding buffer until a baseline UV absorbance was reached monitored at 280 nm. Elution was performed at 0.5 ml/min with elution buffer (0.1 M glycine pH 3.1) and samples were collected in 1.5 ml Eppendorf tubes, in 200 μl fractions. Immediately after elution, samples were neutralized with 1 M Tris pH 11, already pipetted into wells for elution. After purification, eluted samples were dialyzed and concentrated against PBS using Amicon columns.

Afterwards, purified antibodies were analyzed on SDS-PAGE (coomassie stain) and in WB. Protein concentrations of dialyzed samples were determined using a PIERCE BCA kit. Aliquots of antibodies were stored for long term at −80° C. To re-use the protein-G sepharose column, the resin was regenerated by washing first with 6 M Urea, washing and flushing with 20% ethanol and afterwards stored at +4° C. Each HiTrap® Protein G HP GE Healthcare 1 ml column was used monoclonal antibody-specific, always the same column for the same hybridoma.

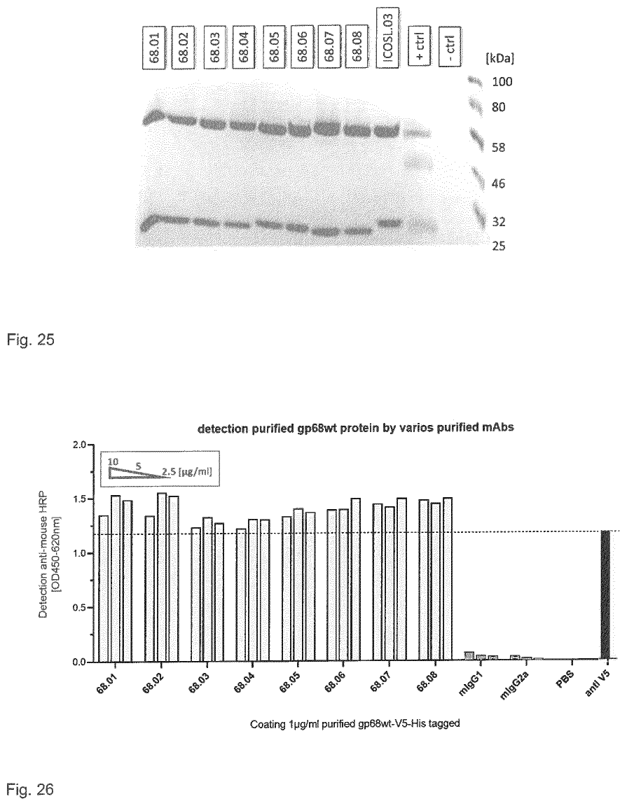

FIG. 25 shows purified anti-vFcγR gp68 mAbs. Exemplarily, all generated anti-gp68 mAbs after NiNTA purification from hybridoma cell culture supernatants under starvation conditions (no FCS) were analyzed in coomassie stain. 5 μg of each mAb was loaded on SDS-page and stained. In addition, one representative mAb raised against recombinant, soluble human ICOSL (ICOSL.03) was investigated. Commercial available mouse IgG1 isotype antibody (Invitrogen) served as positive control and cell lysate from non-antibody producing cells (SP2/0 hybridoma cells) were loaded as negative control.

### Characterization mAbs

### Detection of vFcγRs gp68 in ELISA Using mAbs

To further characterize newly generated mAbs raised against vFcγRs gp34 and gp68, purified mAbs were tested for binding and recognition of the appropriate antigen, e.g., gp34 or gp68. First, mAbs were tested on immobilized recombinant, soluble gp68-V5-His protein that was used for immunization. The antigen was immobilized on ELISA plates and mAb in a dose-dependent manner were added and allowed for binding. Bound anti-gp68 specific mAbs were detected using goat-anti-mouse IgG-HRP secondary antibodies. ELISA results confirmed specific recognition of gp68 antigen by all generated anti-gp68 mAbs.

FIG. 26 shows the detection of immobilized sgp68-V5-His by various anti-gp68 mAbs. Purified anti-gp68 mAbs were further analyzed for binding towards sgp68-V5-His protein in a dose-dependent manner. 10 μg/ml, 5 μg/ml or 2.5 μg/ml of each mAb was used for the detection of 1 μg/ml immobilized recombinant, soluble gp68 protein in ELISA. Commercially available mouse IgG1 and IgG2a isotype antibody (Invitrogen) was used as negative control, as well as PBS only. Using an antibody detecting the V5-Tag on sgp68-V5-His served as a positive control for loading of coated antigen. All anti-gp68 mAbs specifically and similar recognize recombinant vFcγR gp68.

To determine differences among those mAbs ELISA-binding assays were repeated with further titration of anti-gp68 mAbs to identify most potent of them with regard to gp68 recognition.

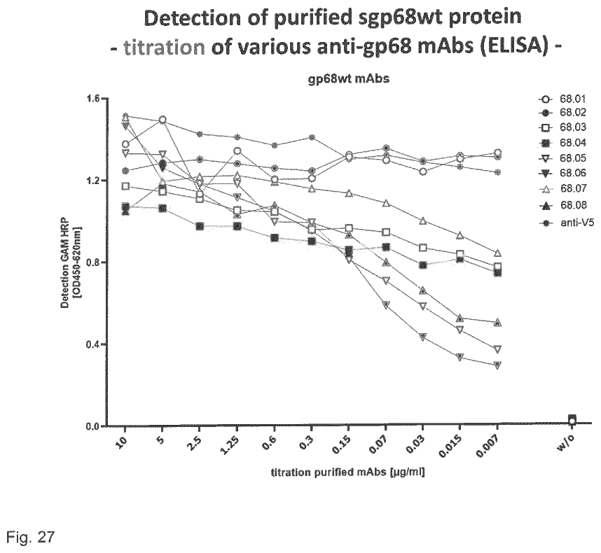

FIG. 27 shows the titration anti-gp68 mAbs on immobilized recombinant sgp68. Purified anti-gp68 mAbs were further analyzed for binding towards sgp68-V5-His protein in titration. 10 μg/ml mAbs was further titrated using log2 dilutions over 12 steps (last well without mAbs) for the detection of 1 μg/ml immobilized recombinant, soluble gp68 protein in ELISA. Using an antibody detecting the V5-Tag on sgp68-V5-His served as a positive control for loading of coated antigen. All anti-gp68 mAbs specifically recognize recombinant vFcγR gp68. In addition, now clearly, dose-dependent variations especially in lower concentration become obvious.

Based on those ELISA data mAbs were selected for best binding affinity towards recombinant gp68, efficient recognizing the antigen also in very low mAb concentrations. This may also provide advantages when antigen-concentrations will be at low detection limit.

### Detection of vFcγRs gp34 in ELISA Using mAbs

To further characterize newly generated mAbs, raised against vFcγRs gp34, were tested for binding and recognition of the appropriate recombinant antigen. First, mAbs were tested on immobilized recombinant, soluble gp34mtrp-V5-His protein that was used for immunization. The antigen was immobilized on ELISA plates and mAb in a dose-dependent manner were added and allowed for binding. Bound anti-gp34mtrp specific mAbs were detected using goat-anti-mouse IgG-HRP secondary antibodies. To our surprise, ELISA results confirmed specific recognition of gp34mtrp protein, only by anti-gp34mtrp mAbs. As a reminder, gp34mtrp-mAbs were generated by immunizing mice using the sgp34mtrp. This protein is impaired for IgG-Fc-binding and is structurally restricted to monomers and dimers, lacking the ability to form higher oligomers.

None of the anti-gp34wt mAbs (native sgp34wt was used for immunization) is capable to recognize gp34mtrp protein suggesting that those mAbs recognize a structural determinant of gp34 only formed and accessible by the native gp34 wild-type protein.

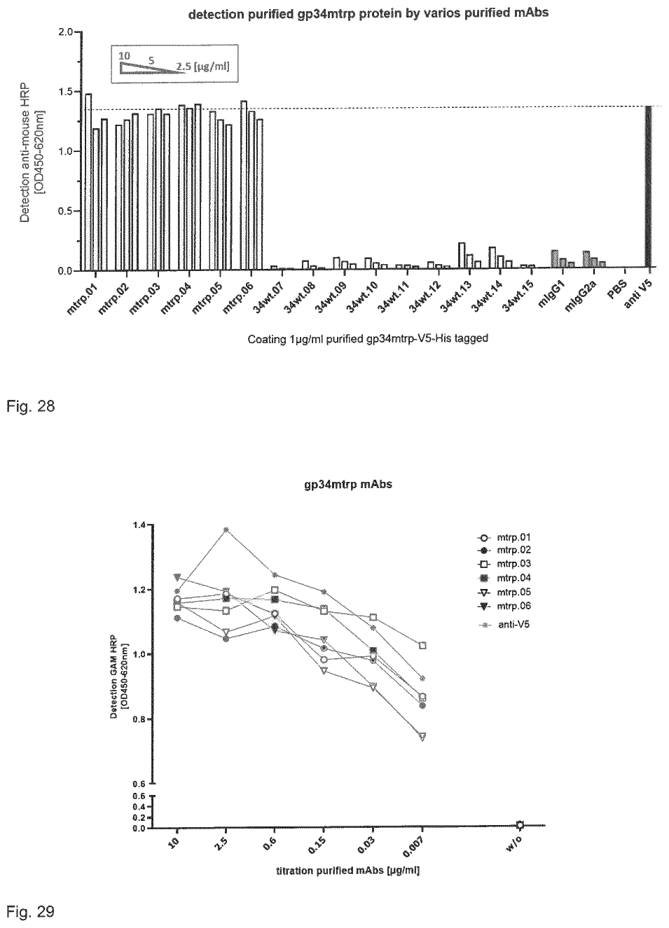

FIG. 28 shows the detection of immobilized sgp34mtrp-V5-His by various anti-gp34wt and anti-gp34mtrp mAbs. Purified anti-gp34 mAbs were further analyzed for binding towards sgp34mtrp-V5-His protein in a dose-dependent manner. 10 μg/ml, 5 μg/ml or 2.5 μg/ml of each mAb was used for the detection of 1 μg/ml immobilized recombinant, soluble gp34mtrp protein in ELISA. Commercially available mouse IgG1 and IgG2a isotype antibody (Invitrogen) was used as negative control, as well as PBS only. Using an antibody detecting the V5-Tag on sgp68-V5-His served as a positive control for loading of coated antigen. All anti-gp34mtrp mAbs specifically and similar recognize recombinant sgp34mtrp protein but none of the anti-gp34wt mAbs recognize sgp34mtrp protein.

To determine differences among those mAbs ELISA-binding assays were repeated with further titration of anti-gp34mtrp mAbs to identify most potent of them with regard to gp34mtrp protein recognition.

FIG. 29 shows the titration of anti-gp34mtrp mAbs on immobilized recombinant sgp34mtrp. Purified anti-gp34mtrp mAbs were further analyzed for binding towards sgp34mtrp-V5-His protein in titration. 10 μg/ml mAbs was further titrated using log4 dilutions over 7 steps (last well without mAbs) for the detection of 1 μg/ml immobilized recombinant, soluble gp34mtrp protein in ELISA. Using an antibody detecting the V5-Tag on sgp34mtrp-V5-His served as a positive control for loading of coated antigen. All anti-gp34mtrp mAbs specifically recognize recombinant sgp34mtrp protein. In addition, now, dose-dependent variations especially in lower concentration become obvious.

To further characterize newly generated mAbs raised against vFcγRs gp34, purified mAbs were tested for binding and recognition of the appropriate recombinant antigen. Next, mAbs were tested on immobilized recombinant, soluble sgp34wt-V5-His protein that was used for immunization. The antigen was immobilized on ELISA plates and mAb in a dose-dependent manner were added and allowed for binding. Bound anti-gp34wt specific mAbs were detected using goat-anti-mouse IgG-HRP secondary antibodies. ELISA data revealed, all anti-gp34mtrp mAbs and all anti-gp34wt mAbs recognize sgp34wt protein. As a reminder, gp34wt-mAbs were generated by immunizing BALB/c mice using the native sgp34wt protein. This protein is fully functional with regard to IgG-Fc-binding and structurally forms monomers, dimers and higher oligomers.

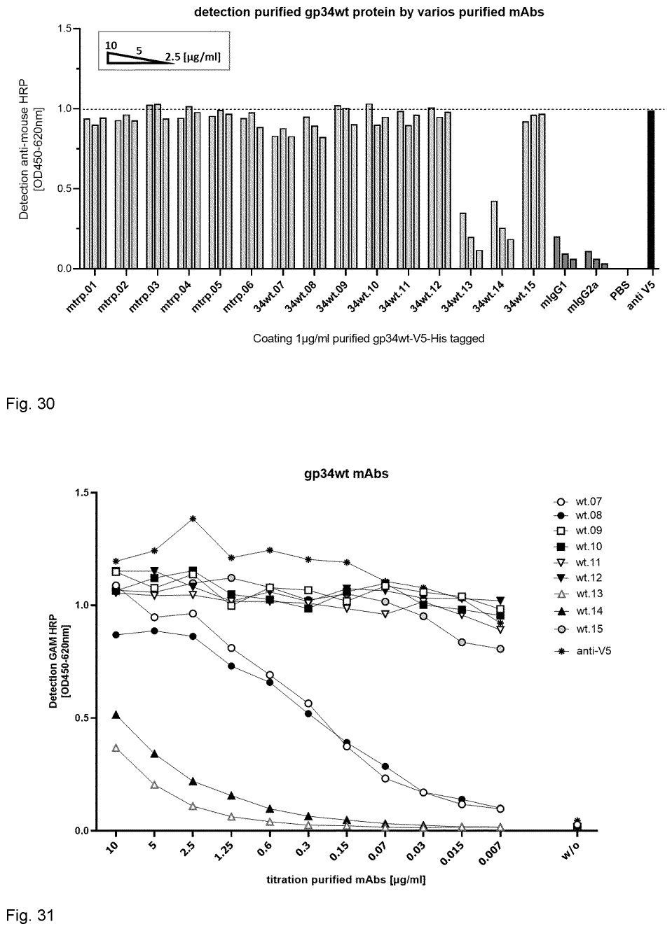

FIG. 30 shows the detection of immobilized sgp34wt-V5-His by various anti-gp34wt and anti-gp34mtrp mAbs. Purified anti-gp34 mAbs were further analyzed for binding towards sgp34wt-V5-His protein in a dose-dependent manner. 10 μg/ml, 5 μg/ml or 2.5 Ξg/ml of each mAb was used for the detection of 1 μg/ml immobilized recombinant, sgp34wt protein in ELISA. Commercially available mouse IgG1 and IgG2a isotype antibody (Invitrogen) was used as negative control, as well as PBS only. Using an antibody detecting the V5-Tag on sgp34wt-V5-His served as a positive control for loading of coated antigen. All anti-gp34mtrp mAbs and all anti-gp34wt mAbs specifically and similar recognize recombinant sgp34wt protein. Only mAbs 34wt.13 (IgG2a subtype) and 34wt.14 (IgG2a subtype) are different and show only low affinity binding towards recombinant sgp34wt protein.

To determine differences among those mAbs, ELISA-binding assays were repeated with further titration of anti-gp34wt mAbs and anti-gp34mtrp mAbs to identify most potent of them with regard to sgp34wt protein recognition.

FIG. 31 illustrates the titration of anti-gp34wt mAbs on immobilized recombinant sgp34wt. Purified anti-gp34wt mAbs were further analyzed for binding towards sgp34wt-V5-His protein in titration. 10 μg/ml mAbs was further titrated using log2 dilutions over 11 steps (last well without mAbs) for the detection of 1ug/ml immobilized recombinant, sgp34wt protein in ELISA. Using an antibody detecting the V5-Tag on sgp34mtrp-V5-His served as a positive control for loading of coated antigen. All anti-gp34wt mAbs specifically recognize recombinant sgp34wt protein. In addition, now drastically, dose-dependent variations especially in lower concentration become obvious. Again, mAbs 34wt.13 (IgG2a subtype) and 34wt.14 (IgG2a subtype) exhibit only low affinity binding towards recombinant sgp34wt protein.

To exclude, differences in recognition are based on immobilization of antigen due to direct protein coating, ELISA binding experiments were repeated and antigens were pre-bound on Nickel-coated plates. Results obtained were the same as in direct-antigen coating ELISAs previously.

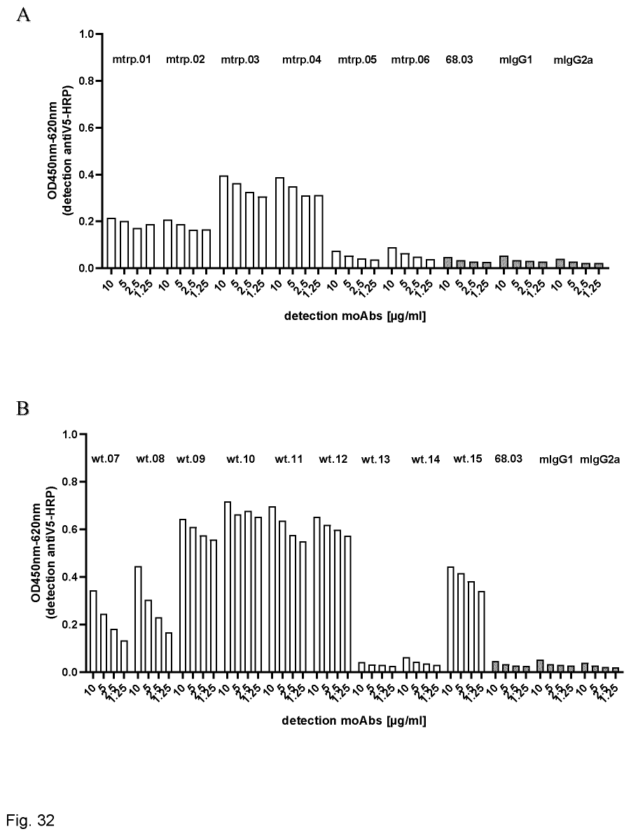

FIG. 32 is the detection of sgp34wt-V5-His on Nickel-coated plates by anti-gp34 mAbs. To exclude structural differences in antigen-presentation, instead of direct coating, sgp34wt-V5-His protein was incubated on Nickel-coated wells. 10 μg/ml, 5 μg/ml or 2.5 μg/ml of each mAb was added for the detection of 1 μg/ml pre-bound recombinant, soluble sgp34wt protein in ELISA. Detection was either performed with mtrp-mAbs (A) or with wt-mAbs (B). Commercially available mouse IgG1 and IgG2a isotype antibody (Invitrogen) was used as negative control, as well as one of the anti-gp68 specific novel mAbs. All tested anti-gp34mtrp mAbs and all anti-gp34wt mAbs specifically recognize recombinant sgp34wt protein, except for mAbs 34wt.13 (IgG2a subtype) and 34wt.14 (IgG2a subtype) as shown before. Especially anti-gp34wt mAbs exhibit broader differences in binding affinities.

Same experiments were done using sgp34mtrp-V5-His tagged protein pre-incubated on Nickel-coated ELISA plates. Similar results were generated as seen in direct antigen coating before. None of the anti-gp34wt mAbs does recognize the mtrp-variant of gp34 confirming that antibodies derived from immunization with the native, multimeric form of vFcγR gp34 recognize epitopes within the protein either not present in the structurally impaired mtrp-variant or those epitopes are hidden under such conditions and therefore not accessible.

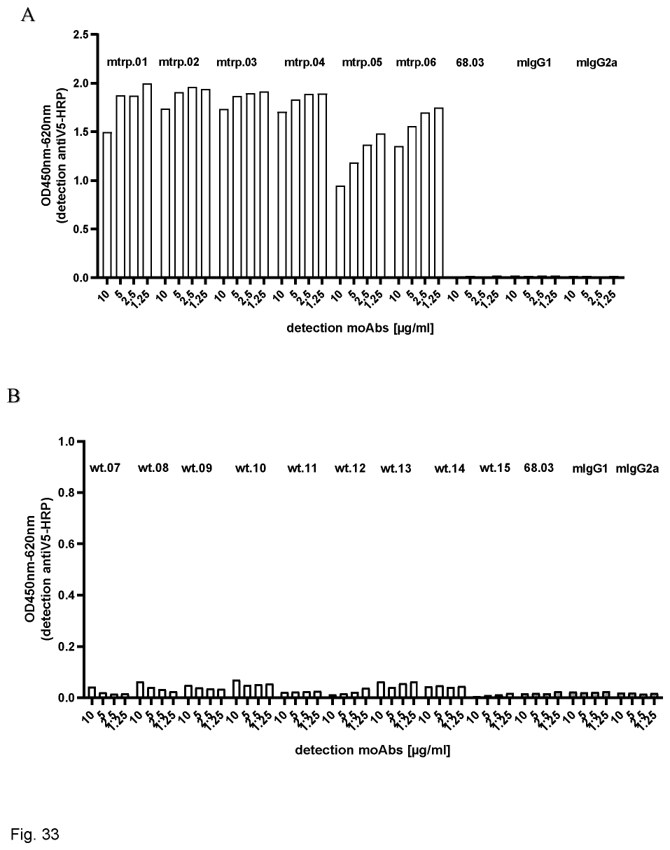

FIG. 33 shows the detection of sgp34mtrp-V5-His on Nickel-coated plates by anti-gp34 mAbs. To exclude structural differences in antigen-presentation, instead of direct coating, sgp34mtrp-V5-His protein was incubated on Nickel-coated wells. 10 μg/ml, 5 μg/ml or 2.5 μg/ml of each mAb was added for the detection of 1 μg/ml pre-bound recombinant, soluble gp34mtrp protein in ELISA. Detection was either performed with mtrp-mAbs (A) or with wt-mAbs (B). Commercially available mouse IgG1 and IgG2a isotype antibody (Invitrogen) was used as negative control, as well as one of the anti-gp68 specific novel mAbs. All tested anti-gp34mtrp mAbs recognize sgp34mtrp bound to Nickel-coated wells. Again, none of the anti-gp34wt mAbs do recognize sgp34mtrp protein, neither in direct-antigen coating nor pre-bound to Nickel-opsonized plates.

### Detection vFcγRs gp68 by mAbs in WB

In addition to functional characterization of novel anti-gp34 and anti-gp68 mAbs for detection of their appropriate antigen (recombinant or in the virus context), mAbs were further tested for specificity in western blot (WB) analysis.

Recombinant sgp68wt protein, or sICOSL as non-binding control protein were separated on SDS-page and western blot was performed as described elsewhere. Membrane-bound antigens were incubated with anti-gp68 mAbs and binding was visualized using goat-anti mouse IgG-HRP secondary antibody and chemiluminescent signals measured by incubation of the membranes in substrate. Results are showing following.

Two mAbs derived originally from the same motherwell (MW) by sub-cloning, showed the strongest signal for western blot detection of recombinant sgp68 protein (used also for immunization), whereas, all the other anti-gp68 mAbs recognize only to a lower level higher molecular weight form of gp68. One mAb (gp68.06) at least in western blot shows only a very weak detection signal if positive at all.

None of the anti-gp68 mAbs recognizes loaded sICOSL control-protein, indicating that those mAbs does bind to the gp68 ECD and not to the V5-His-Tag.

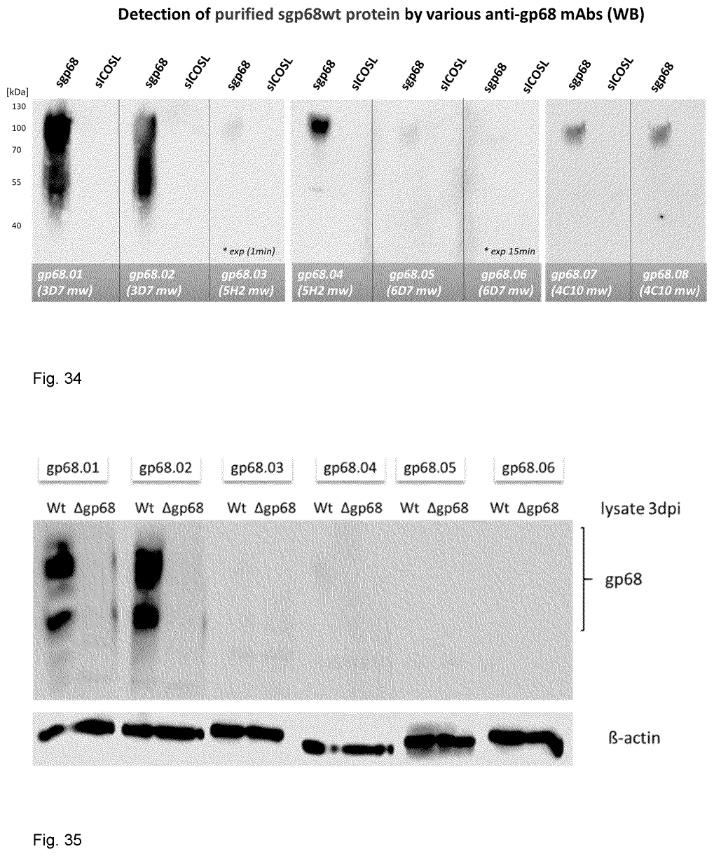

FIG. 34 shows the detection of recombinant sgp68 by anti-gp68 mAbs. Recombinant proteins sgp68wt or sICOSL were separated on SDS-page and transferred onto a membrane for western blot analysis. Membrane pieces containing one lane with sgp68 and one lane sICOSL each were incubated with indicated mAbs raised against vFcγR gp68. mAbs 68.01 and 68.02 show strongest detection signals for recombinant gp68, followed by other anti-gp68 mAbs recognizing at least higher glycosylated form of gp68 in western blot, but with lower signals. None of the anti-gp68 mAbs cross-react with recombinant sICOSL protein.

Western blot analysis were repeated using this time cell lysates from cells infected either with HCMV AD169 wild-type strain or a AD169 virus mutant lacking vFcγR gp68 (Δgp68). In virus-context now, two mAbs are able to detect gp68 also in virus context: gp68.01 and gp68.02. Both mAbs showed also the strongest effect on binding in western blot before using recombinant protein.

FIG. 35 shows the detection of vFcγR gp68 by anti-gp68 mAbs in infection. MRC5 fibroblast were infected with MOI 2. Three days post infection cell lysates were generated. Next, lysates were analyzed in western blot for detection of vFcγR gp68 by novel mAbs. Again, only mAbs 68.01 and 68.02 shows specific detection of gp68 now also in infection, indicating that those two mAbs indeed recognize vFcγR gp68 and can be used for western blot analysis. Membrane pieces containing one lane with lysate from AD169wt infected cells and one lane containing lysate from AD169 Agp68 infected cells were incubated with indicated mAbs. As observed before, only mAbs 68.01 and 68.02 detect vFcγR gp68 in western blot. No detection is observed in lysates lacking gp68 demonstrating specificity of those mAbs. ß-actin stain was performed as loading control, indicating that comparable amounts of cell lysates were loaded onto the gel.

### Detection by Immuno-Precipitation Using Lysates of HCMV-Infected Cells

Finally, anti-vFcγR gp68 mAbs were used for immunoprecipitation experiments. MRC5 fibroblasts were infected with HCMV AD169wt strain or a virus mutant lacking vFcγR gp68 (Δgp68) for 4 days with MOI 3. Infected cells were metabolically labeled with ³⁵S for 2 hours at 37° C., washed and lysed using NP40-lysis buffer. Lysates were incubated with indicated mAbs or human IgG Fc-fragment to pulldown vFcγR gp68 specifically or both HCMV-encoded vFcγRs gp34, gp68 using human IgG-Fc-fragment to precipitated Fc-binding proteins. Samples were deglycosylated by EndoH and separated on SDS-page gradient gel. In summary, all anti-gp68 mAbs recognize and bind vFcγR gp68 from infected cells. To some extend also vFcγR gp34 is detected probably due to the strong binding of vFcγR gp34 towards mouse IgGs, stronger to mouse IgG1 than to mouse IgG2a (68.07 and 68.08). Therefore this small background binding to gp34 is absent when using 68.07 and 68.08 mAbs.

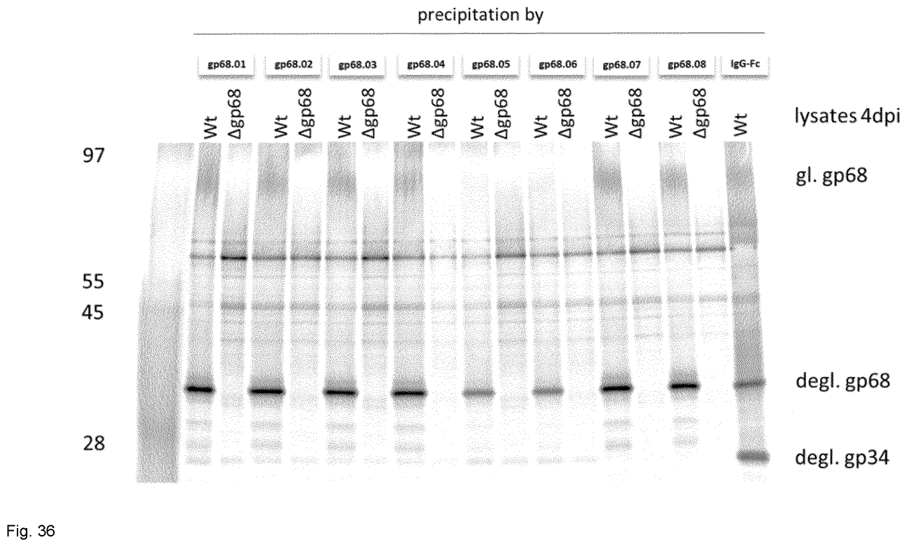

FIG. 36 shows the precipitation of vFcγR gp68 from infected cells by anti-gp68 mAbs. MRC5 fibroblast were infected with MOI 2. Four days post infection cells were metabolically labelled and following lysates were incubated with indicated anti-gp68 mAbs or human IgG-Fc-fragment for precipitation of vFcγR gp68. Indeed, all anti-gp68 mAbs precipitated vFcγR gp68 from AD169 infected cells. Signals were absent in lysates lacking vFcγR gp68 demonstrating specificity. Except for mAbs 68.05 and 68.06 also EndoH-resistant, glycosylated gp68.

### Detection vFcγR gp34 by Various mAbs in WB

In addition to functional characterization of novel anti-gp34wt- and anti-gp34mtrp-mAbs for detection of their appropriate antigen in ELISA, mAbs were further tested for their specificity in western blot (WB) analysis. Recombinant sgp34wt or sgp34mtrp protein were loaded onto SDS-PAGE and detected in western blot using anti-gp34mtrp mAbs. Most of them (mtrp.01-mtrp.04) recognize sgp34wt and gp34mtrp protein, including dimers for the sgp34wt protein. mAbs mtrp.05 and mtrp.06 are less efficient to recognize sgp34wt/mtrp protein in western blot. Results confirm binding affinities seen before in ELISA.

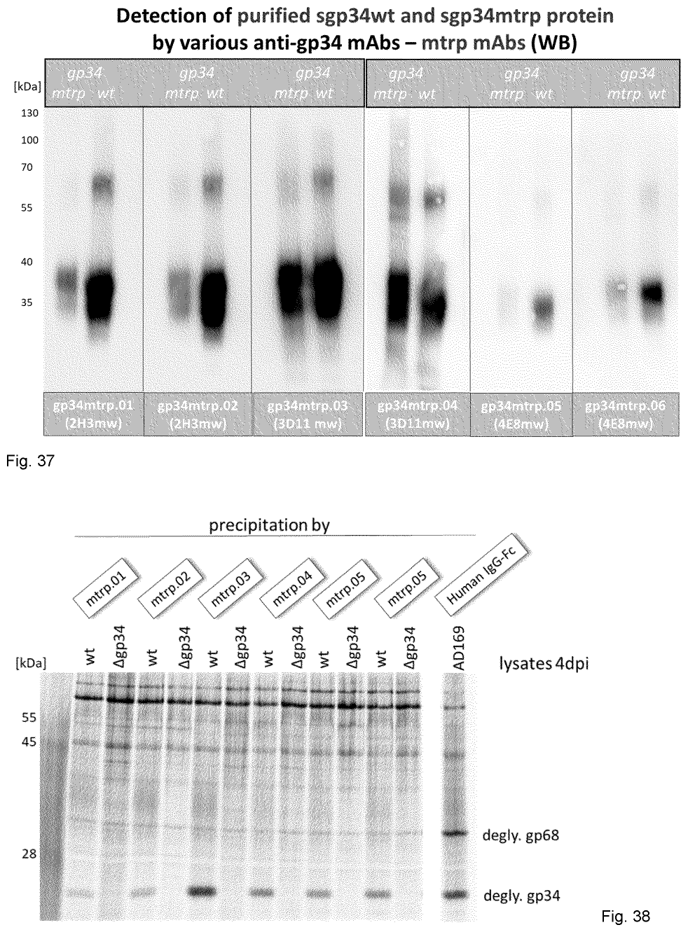

FIG. 37 shows the detection of recombinant sgp34wt or sgp34mtrp by anti-gp34 mAbs. Recombinant proteins sgp68 or sICOSL were separated on SDS-page and transferred onto a membrane for western blot analysis. Membrane pieces containing one lane with sgp68 and one lane sICOSL each were incubated with indicated mAbs raised against vFcγR gp68. mAbs 68.01 and 68.02 show strongest detection signals for recombinant gp68, followed by other anti-gp68 mAbs recognizing at least higher glycosylated form of gp68 in western blot, but with lower signals. None of the anti-gp68 mAbs cross-react with recombinant sICOSL protein.

In addition, mAbs raised against gp34mtrp were used for precipitation of vFcγR gp34 from infected cells. For comparison and to demonstrate specificity, lysates from HCMV AD169 wild-type cells were used as well as from cells infected with a HCMV AD169 mutant lacking vFcγR gp34 (Δgp34). Although not to similar levels, all mAbs raised against sgp34mtrp protein are able to recognize vFcγR gp34 in infected cells.

FIG. 38 shows the precipitation of vFcγR gp34 from infected cells by anti-gp34mtrp mAbs. MRC5 fibroblast were infected with MOI 2. Four days post infection cells were metabolically labelled and following lysates were incubated with indicated anti-gp34mtrp mAbs for precipitation of vFcγR gp34 or human IgG-Fc-fragment for precipitation of both, vFcγRs gp34 and gp68. Indeed, all anti-gp34mtrp mAbs efficiently precipitated vFcγR gp34 from HCMV AD169 infected cells. Signals were absent in lysates lacking vFcγR gp34 demonstrating specificity.

Next, mAbs raised against sgp34wt protein were investigated for their specificity in western blot (WB) analysis. Recombinant sgp34wt, sgp34mtrp or sICOSL (non-binding control) protein were loaded onto SDS-PAGE and detected in western blot using anti-gp34wt mAbs. Most of them (except mAb 34wt.15) recognize sgp34wt protein; again, none of them is able to detect sgp34mtrp protein. In addition, no unspecific binding on membranes loaded with sICOSL protein was observed. Positive control for proteins used in this experiment was done using anti-His antibodies or one of the functional anti-gp34mtrp mAbs from before. Results confirm binding affinities seen before in ELISA with some interesting exceptions. First, mAbs exhibits strong binding in ELISA 34wt.15, but at least in this assay was not working. This needs to be further investigated. In addition, mAbs wt.13 and wt.14 showed only weak detection signals in ELISA but seem to be comparable with other mAbs in this western blot.

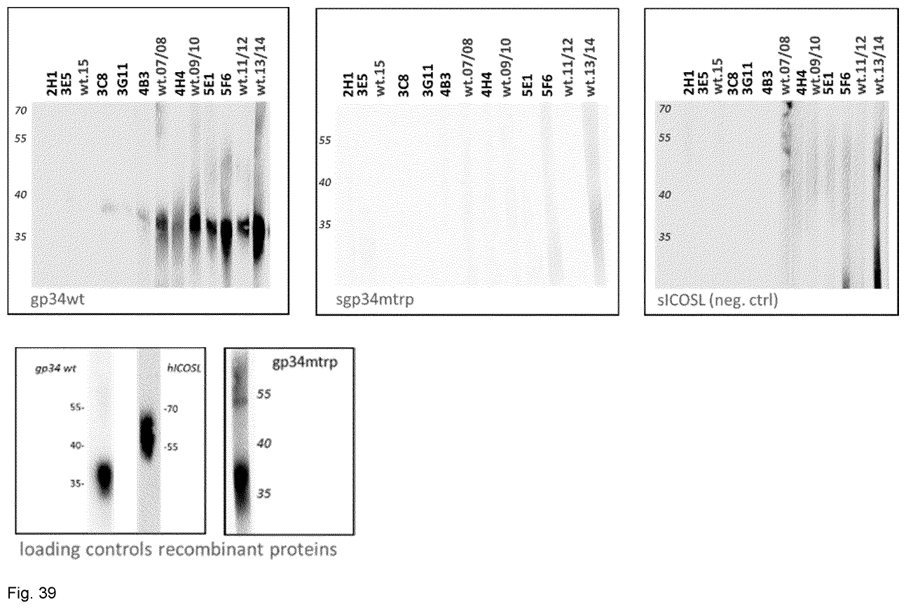

FIG. 39: detection of recombinant sgp34wt or sgp34mtrp by anti-gp34wt mAbs. Recombinant proteins sgp34wt, sgp34mtrp or sICOSL were separated on SDS-page and transferred onto a membrane for western blot analysis. Membrane lanes containing recombinant proteins were stained with indicated SN from hybridomas of mAbs. Results verified results of ELISA binding studies: mAbs anti-gp34wt do not recognize recombinant sgp34mtp protein.

Next, regions within the antigen were determined that are recognized by appropriate mAbs. For this purpose, truncation variants of sgp34wt protein ECD were transiently transfected into HEK-293T cells. One day after transfection, cells were metabolically labeled by ³⁵S, 2 hrs 37° C., lysed and precipitation experiments were done incubating indicated lysates with indicated anti-gp34mtrp mAbs. To the end, three versions of sgp34wt ECD were used: (i) sgp34wt protein consisting of wildtype sequence covering amino acids 1-179 (complete ECD; used for immunization), (ii) sgp34wt protein consisting of wildtype sequence covering amino acids 1-156 (losing two predicted O-glycosylation sites) and (iii) sgp34wt protein consisting of wildtype sequence covering amino acids 1-124 (complete Ig-like domain). Previous studies by our group demonstrated, that the region necessarily for IgG-Fc-binding involves amino acids of the ECD including complete Ig-like domain and further amino acids. IgG-Fc-binding experiments done earlier confirmed, that sgp34wt (1-124aa) does not bind any longer to IgG-Fc.

The results indicate at least (i) mAbs mtrp.01 and mtrp.02 recognize all three soluble variants of sgp34wt, (ii) mAbs mtrp.03 and mtrp.04 show strongest binding to the variant of sgp34wt containing the complete ECD, but do not recognize the variant covering amino acids 1-124 and (iii) mAbs mtrp.05 and mtrp.06 show similar binding to the variants of sgp34wt containing 1-156aa and also 1-179aa, but also do not recognize the variant covering amino acids 1-124.

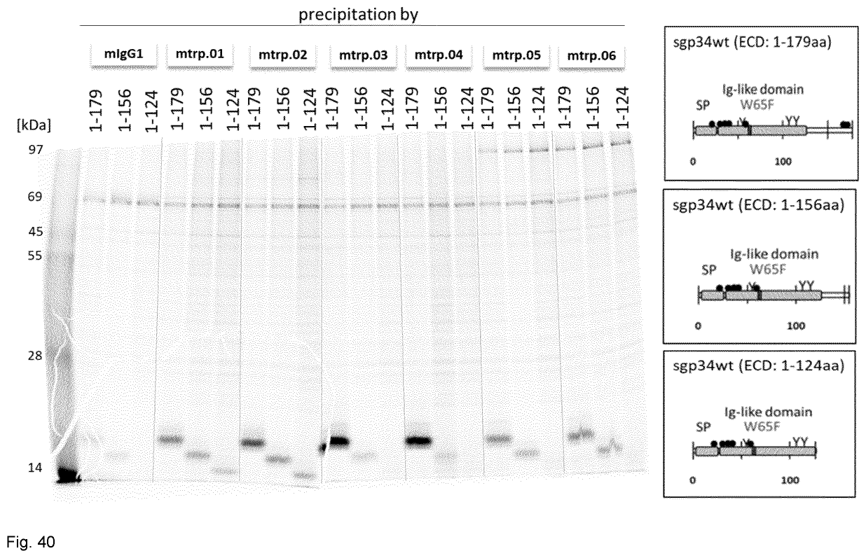

FIG. 40: epitope mapping of mAbs anti-gp34mtrp. To further determine binding epitopes recognized by various anti-gp34mtrp mAbs, truncated versions of sgp34wt protein were transiently transfected in HEK-293-T cells. One day post transfection cells were metabolically labeled with 35S and lysates used for precipitation by indicated mAbs or mouse IgG1 isotype control. Samples were separated on SDS-gradient gel and analyzed on phosphoplate. Results confirmed data from ELISA binding studies: all mAbs anti-gp34mtrp recognize sgp34wt protein, whereby mtrp.01 and mtrp.02 in addition also recognize the shortest truncation variant comprising amino acids 1-124.

In summary, precipitation experiments confirmed again, that all anti-gp34mtrp mAbs recognize recombinant sgp34wt protein and can be used for pulldown experiments.

In line with that, we also wanted to determine regions within the antigen that are recognized by anti-gp34wt mAbs. As before, truncation variants of sgp34wt protein ECD were transiently transfected into HEK-293T cells. One day after transfection, cells were metabolically labeled by ³⁵S, 2 hrs 37° C., lysed and precipitation experiments were done incubating indicated lysates with indicated anti-gp34wt mAbs. To the end, three versions of sgp34wt ECD were used: (i) sgp34wt protein consisting of wildtype sequence covering amino acids 1-179 (complete ECD; used for immunization), (ii) sgp34wt protein consisting of wildtype sequence covering amino acids 1-156 (losing two predicted O-glycosylation sites) and (iii) sgp34wt protein consisting of wildtype sequence covering amino acids 1-124 (complete Ig-like domain). Previous studies by our group demonstrated, that the region necessarily for IgG-Fc-binding involves amino acids of the ECD including complete Ig-like domain and further amino acids. IgG-Fc-binding experiments done earlier confirmed, that sgp34wt (1-124aa) does not bind any longer to IgG-Fc.

The results indicate at least (i) all anti-gp34wt mAbs recognize sgp34wt variants including amino acids 1-156 and the amino acids 1-179, (ii) all mAbs detect the recombinant soluble truncation variants of sgp34wt protein to a similar extend, except mAbs wt.13 and wt.14 (both mouse IgG2a) and (iii) none of the anti-gp34wt mAbs precipitated truncation variant 1-124 amino acids.

Interesting, one can clearly see non-specific precipitation by the mouse IgG1 subtype, demonstrating remaining binding capacity of vFcγR gp34 to mouse IgG1 as mentioned earlier.

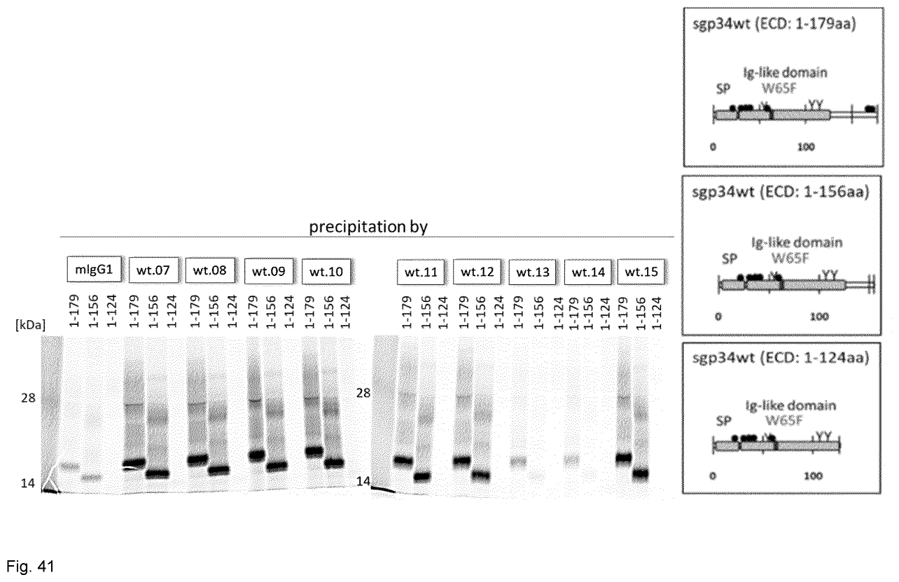

FIG. 41: epitope mapping of mAbs anti-gp34wt. To further determine binding epitopes recognized by various anti-gp34wt mAbs, truncated versions of sgp34wt protein were transiently transfected in HEK-293-T cells. One day post transfection cells were metabolically labeled with ³⁵S and lysates used for precipitation by indicated mAbs or mouse IgG1 isotype control. Samples were separated on SDS-gradient gel and analyzed on phosphoplate. Results confirmed data from ELISA binding studies: all mAbs anti-gp34wt recognize sgp34wt protein, whereby wt.13 and wt.14 exhibit weakest interaction. None of the mAbs recognizes the shortest truncation variant, comprising amino acids 1-124.

### Blocking Capacity of Anti-vFcγR gp34 and gp68 mAbs

Glycoproteins gp34 and gp68 are HCMV encoded type-I transmembrane molecules able to bind to the Fc-part of human immunoglobulin G (IgG).

By this, gp34 and gp68 can interact with IgG, as their human counterparts the human FcγRs. Through this interaction, they antagonize IgG-dependent host FcγR activation e.g. ADCC mediated by FcγRIII+-NK cells.

Since IgG-binding by vFcγRs gp34 and gp68 is mandatory for their inhibitory effect on host FcγRs we next wanted to identify the ability of our novel generated anti-vFcγR mAbs with respect to their capacity to block IgG binding.

### IgG-Blocking Capacity of Anti-vFcγR gp68-Specific mAbs

To test if anti-gp68 specific mAbs can interfere with IgG-binding ability of gp68 and human IgG. Therefore, recombinant, soluble gp68 was pre-incubated with indicated anti-gp68 specific mAbs, mouse IgG isotype or PBS only. Next, antibody bound sgp68 was incubated on IgG-immobilized ELISA plates. IgG-binding by sgp68 in presence or absence of potential blocking mAbs was determined detecting bound glycoprotein sgp68-V5-His using anti-V5 antibody.

Results indicated, that all anti-gp68 mAbs dose-dependently block IgG-binding ability of sgp68 in the following hierarchy: 68.01>68.03>68.07/68.08/68.04>68.02/68.05/68.06.

Incubation with mouse IgG1 and IgG2a isotypes only, did not influence IgG-binding by sgp68.

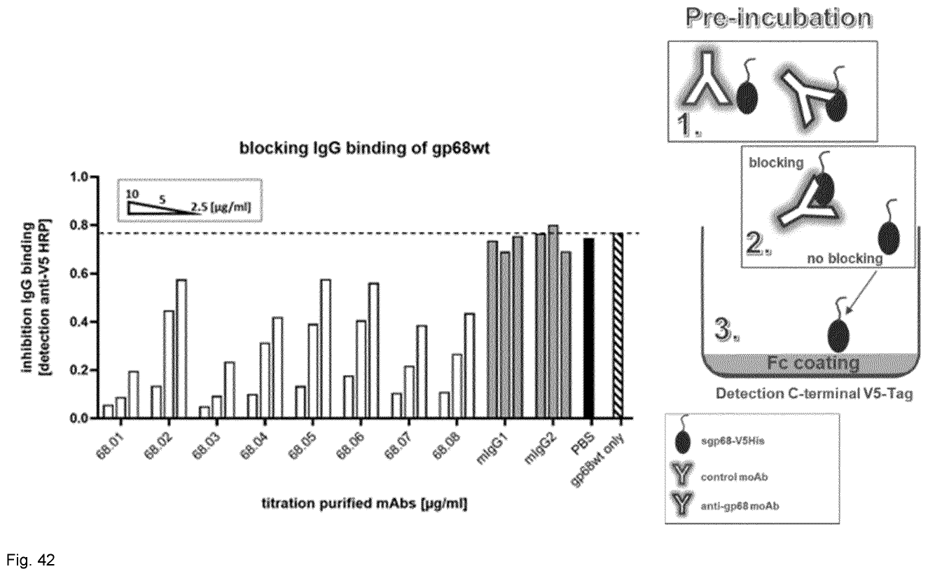

FIG. 42: IgG blocking ability by anti-gp68 mAbs. vFcγR gp68 is an HCMV encoded IgG-Fc-binding. mAbs were pre-incubated with recombinant, sgp68 and next added to IgG-immobilized ELISA plates. Bound sgp68 in absence or presence of blocking mAbs is detected using anti-V5-HRP antibody recognizing IgG-bound sgp68-V5-His. All anti-gp68 specific mAbs have the potential to block vFcγR gp68 IgG-binding in a dose-dependent manner. Effects by non-specific mouse IgG isotypes are indicated.

Finally, summarized in table 6 (Tab. 6), characteristics and potentials of anti-vFcγR gp68-specific mAbs are highlighted.

In the end, 8 anti-gp68 mAbs were provided, all of them specifically recognizing vFcγR gp68as soluble, recombinant protein but also in lysates of HCMV AD169 infected cells. More important, all of them have the potential to interfere with IgG-binding ability of gp68.

TABLE 6
Summary anti-gp68 moAbs
Summarizing properties of anti-gp68 mAbs. We generated
in total 8 anti-gp68-specific mAbs and original
motherwells (MW) are indicated from hybridomas further
sub-cloned by limiting dilutions. mAbs were
characterized in ELISA, WB and precipitation experiments.
In addition, all of them can block IgG binding
by gp68. For further purposes, mAbs are recombinant cloned
and sequences are determined including framework
regions (FR) and complementarity-determining regions (CDRs).

| | | | | | | | IgG |
|---|---|---|---|---|---|---|---|
| | MW | subclass | SEQ CDRs | ELISA | WB | IP | Blocking |
| gp68.01 | 3D7 | mlgG1 | + | +++ | +++ | ++ | +++ |
| gp68.02 | 3D7 | mlgG1 | + | +++ | +++ | ++ | + |
| gp68.03 | 5H2 | mlgG1 | + | +++ | + | ++ | +++ |
| gp68.04 | 5H2 | mlgG1 | + | +++ | ++ | ++ | ++ |
| gp68.05 | 6D7 | mlgG1 | + | ++ | − | + | + |
| gp68.06 | 6D7 | mlgG1 | + | ++ | − | + | + |
| gp68.07 | 4C10 | mlgG2a | + | +++ | ++ | ++ | ++ |
| gp68.08 | 4C10 | mlgG2a | + | ++ | ++ | ++ | ++ |

### IgG-Blocking Capacity of Anti-vFcγR gp34-Specific mAbs

First, binding capacity by various anti-gp34wt and anti-gp34mtrp mAbs towards recombinant sgp34wt protein will be recapitulated. Therefore, constant immobilized sgp34wt protein detection by constant concentration by indicated mAbs was measured in ELISA, highlighting differences upon antigen binding that might also affect blocking capacities. Bound mAbs were detected using goat anti-mouse IgG-HRP secondary antibody.

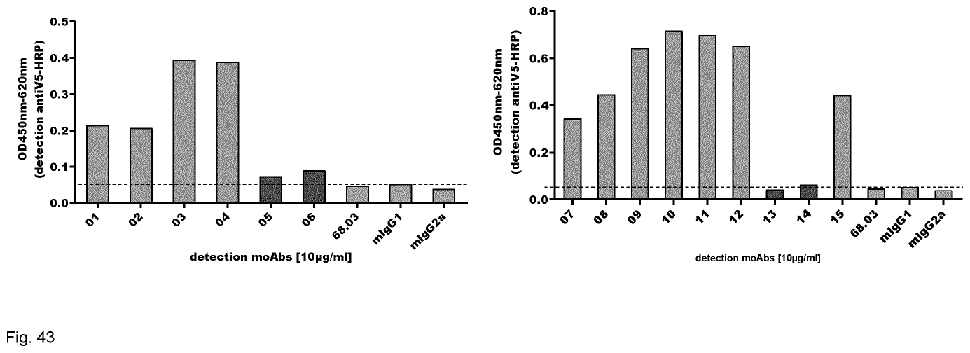

FIG. 43 shows the detection of immobilized sgp34wt-V5-His by various anti-gp34wt and anti-gp34mtrp mAbs. Purified anti-gp34 mAbs (both subgroups in comparison) at 10 μg/ml concentration were analyzed for binding of 1 μg/ml immobilized sgp34wt-V5-His protein in ELISA. Commercially available mouse IgG1 and IgG2a isotype antibody (Invitrogen) were used as negative control, as well as one anti-gp68-specific mAbs. Tested anti-gp34mtrp mAbs and all anti-gp34wt mAbs specifically recognize recombinant sgp34wt protein, except wt.13 and wt.14 and only to some less extend mtrp.05 and mtrp.06.

First, the blocking potential of anti-gp34mtrp mAbs (mtrp.01-mtrp.06) was investigated and afterwards anti-gp34wt mAbs (wt.07-wt.15).

To test if anti-gp34mtrp specific mAbs can interfere with IgG-binding ability of gp34 and human IgG, recombinant, sgp34wt (gp34mtrp is impaired for IgG-Fc-binding) was pre-incubated with indicated anti-gp34mtrp specific mAbs, mouse IgG isotype or PBS only. Next, antibody bound sgp34wt was incubated on IgG-immobilized ELISA plates. IgG-binding by sgp34wt in presence or absence of potential blocking mAbs was determined detecting bound glycoprotein sgp34wt-V5-His using anti-V5 antibody.

Results indicated, not all but some anti-gp34mtrp mAbs dose-dependently can block IgG-binding ability of sgp34wt in the following hierarchy: mtrp.03/mtrp.04>mtrp.01/mtrp.02>mtrp.05/mtrp.06.

Incubation with mouse IgG1 affects to some extend sgp34wt binding to immobilized human IgG, since gp34wt also shows binding ability predominantly to mouse IgG1. Mouse IgG2a isotype only, did not influence IgG binding by sgp34wt. Same holds true for PBS only. Nethertheless, blocking potential of anti-gp34mtrp mAbs is much stronger compared to mouse IgG1 isotype alone.

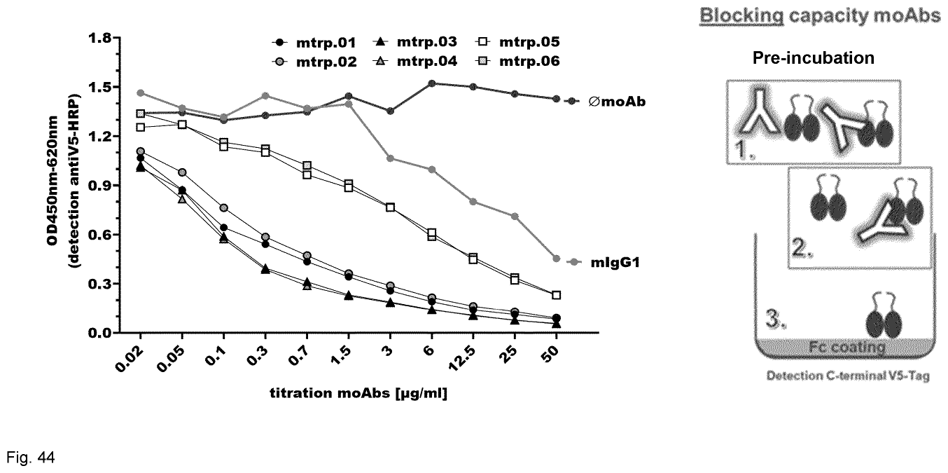

FIG. 44: IgG blocking ability by anti-gp34mtrp mAbs. vFcγR gp34 is an HCMV encoded IgG-Fc-binding. Anti-gp34mtrp-specific mAbs (in titration) were pre-incubated with recombinant, sgp34wt (constant amount 1 μg/ml) and added to IgG-immobilized ELISA plates. Bound sgp34wt in absence or presence of blocking mAbs is detected using anti-V5-HRP antibody recognizing IgG-bound sgp34wt-V5-His. All anti-gp34mtrp-specific mAbs have the potential to block vFcγR gp34 IgG-binding but with variations in efficiency. Dose-dependent potential of mAbs used for blocking is indicated. Effect by non-specific mouse IgG isotype IgG1 is shown. In addition, binding of sgp34wt to immobilized IgG in absence of any mAb is depicted.

Next, the blocking potential of anti-gp34wt mAbs (wt.07-wt.15) was investigated. To test if anti-gp34wt specific mAbs can interfere with IgG-binding ability of gp34 and human IgG. Therefore, recombinant, sgp34wt was pre-incubated with indicated anti-gp34wt specific mAbs, mouse IgG isotype or PBS only. Next, antibody bound sgp34wt was incubated on IgG-immobilized ELISA plates. IgG-binding by sgp34wt in presence or absence of potential blocking mAbs was determined detecting bound glycoprotein sgp34wt-V5-His using anti-V5 antibody.

Results indicated, not all but some anti-gp34wt mAbs dose-dependently can block IgG-binding ability of gp34 in the following hierarchy: wt.09/wt.10/wt.11/wt.12>wt.15>wt.07/wt.08.

mAbs wt.13 and wt.14 (both mouse IgG2a) does not significantly influence sgp34wt binding to immobilized IgG, even though mouse IgG1 shows stronger effects.

Incubation with mouse IgG1 affects to some extend sgp34wt binding to immobilized human IgG, since gp34wt also shows binding ability predominantly to mouse IgG1. Mouse IgG2a isotype only, did not influence IgG-binding by sgp34wt. Same holds true for PBS only. Nevertheless, blocking potential of anti-gp34wt mAbs is much stronger compared to mouse IgG1 isotype alone.

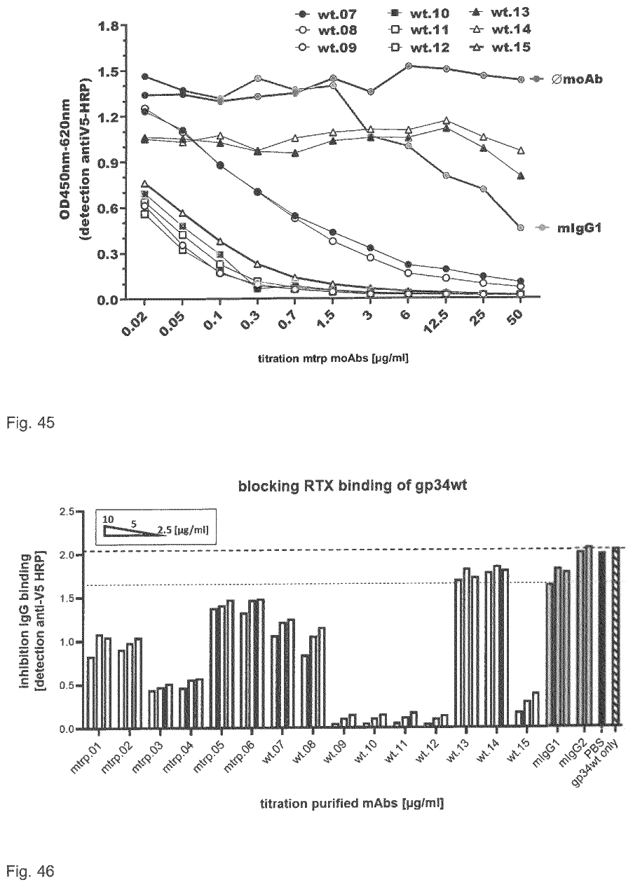

FIG. 45 shows the IgG blocking ability by anti-gp34wt mAbs. vFcγR gp34 is an HCMV encoded IgG-Fc-binding. Anti-gp34wt-specific mAbs (in titration) were pre-incubated with recombinant, sgp34wt (constant amount 1 μg/ml) and added to IgG-immobilized ELISA plates. Bound sgp34wt in absence or presence of blocking mAbs is detected using anti-V5-HRP antibody recognizing IgG-bound sgp34wt-V5-His. Most anti-gp34wt-specific mAbs have the potential to block vFcγR gp34 IgG-binding but with strong variations in efficiency. Dose-dependent potential of mAbs used for blocking is indicated. Also, effect of non-specific mouse IgG1 isotype is shown, highlighting that mAbs wt.13 and wt.14 cannot block sgp34wt-IgG-binding. In addition, binding of sgp34wt to immobilized IgG in absence of any mAb is depicted.

Finally, IC50 values for blocking capacities by various anti-gp34 mAbs are calculated and summarized in table 7 (Tab. 7).

TABLE 7
Summarizing blocking potential of anti-gp34 mAbs illustrated as
IC50 values. IC50 Values, indicating mAb concentration necessary
to inhibit at least 50% IgG-binding by sgp34wt calculated from
mAb titration experiments shown before. For comparison, Infuence
on human IgG binding by agp34wt is indicated as reference.

| | IC50 [ng/ml] |
|---|---|
| mtrp.01 | 239 |
| mtrp.02 | 330 |
| mtrp.03 | 152 |
| mtrp.04 | 147 |
| mtrp.05 | 5338 |
| mtrp.06 | 4456 |
| mtIgG1 | 9690 |
| wt.07 | 447 |
| wt.08 | 494 |
| wt.09 | 63 |
| wt.10 | 89 |
| wt.11 | 77 |
| wt.12 | 70 |
| wt.13 | NB |
| wt.14 | NB |
| wt.15 | 120 |
| mtIgG1 | 9690 |

*NB—non blocking

Finally, summarized in Tab. 8, characteristics and potentials of anti-vFcγR gp68-specific mAbs are highlighted.

In the end, we generated 15 anti-gp34wt mAbs (6 mAbs raised against sgp34mtrp protein: anti-gp34mtrp mAbs and 9 mAbs raised against sgp34wt protein: anti-gp34wt mAbs).

All of them specifically recognized vFcγR gp34wt as soluble, recombinant protein but also in lysates of cells infected with HCMV AD169 strain. Most importantly, all of them have the potential to interfere with IgG-binding ability of gp34wt.

Especially anti-gp34wt mAbs wt.09, wt.10, wt.11, wt.12 and wt.15 show most promising blocking potential to interfere with vFcγR gp34 binding towards human IgG.

TABLE 8
Summary anti-gp34 mAbs
Summarizing properties of anti-gp34 mAbs.
We generated in total 15 anti-gp34-specific mAbs and
original motherwells (MW) are indicated from hybridomas
further sub-cloned by limiting dilution.
mAbs anti-gp34mtrp (immunization with sgp34mtrp protein)
and mAbs anti-gp34 wt (immunization with
sgp34 wt protein) were further characterized in ELISA,
WB and precipitation experiments. In addition,
all of them were tested upon their potential to block gp34
wt IgG-binding resulting in candidates showing
promising potential to interfere with IgG-binding ability of
gp34 wt but also including mAbs that cannot
block IgG-binding by gp34. For further purposes, mAbs are
recombinant cloned and sequences are determined
including framework regions (FR) and
complementarity-determining regions (CDRs).

| | | | | ELISA | ELISA | WB | WB | IP on | |
|---|---|---|---|---|---|---|---|---|---|
| | MW | subclass | SEQ CDRs | 34 wt | mtrp | 34 wt | mtrp | HCMV | IgG Blocking |
| mtrp.01 | 2H3 | mlgG1 | + | +++ | +++ | ++ | ++ | + | + |
| mtrp.02 | 2H3 | mlgG1 | + | +++ | +++ | ++ | ++ | + | + |
| mtrp.03 | 3D11 | mlgG1 | + | +++ | +++ | ++ | ++ | ++ | ++ |
| mtrp.04 | 3D11 | mlgG1 | + | +++ | +++ | ++ | ++ | + | ++ |
| mtrp.05 | 4E8 | mlgG1 | + | +++ | +++ | + | (+) | + | (+) |
| mtrp.06 | 4E8 | mlgG1 | + | +++ | +++ | + | (+) | + | (+) |
| wt. 07 | 4B4 | mlgG1 | + | +++ | − | ++ | − | + | (+) |
| wt. 08 | 4B4 | mlgG1 | + | +++ | − | ++ | − | n.d. | (+) |
| wt. 09 | 4H7 | mlgG1 | + | +++ | − | ++ | − | n.d. | +++ |
| wt. 10 | 4H7 | mlgG1 | + | +++ | − | ++ | − | n.d. | +++ |
| wt. 11 | SA11 | mlgG1 | + | +++ | − | ++ | − | n.d. | +++ |
| wt. 12 | SA11 | mlgG1 | + | +++ | − | ++ | − | n.d. | +++ |
| wt. 13 | 6E3 | mlgG2a | + | + | − | ++ | − | n.d. | − |
| wt. 14 | 6E3 | mlgG2a | + | + | − | ++ | − | n.d. | − |
| wt. 15 | 3F5 | mlgG1 | + | +++ | − | − | − | n.d. | +++ |

Only recently, we identified additional interaction partners of vFcγR gp34: (i) interaction with the membrane-bound B-cell receptor (BCR) of IgG+ memory B-cells, (ii) C-reactive protein (CRP and (iii) the neonatal Fc-Receptor (FcRn). Interaction and binding of vFcγR gp34 was demonstrated earlier in this study. While in the beginning mAbs anti-vFcγR gp68 and gp34 were generated to potentially be able to block IgG-mediated antagonization by these molecules, we tested them also now for their ability to interact with gp34-CRP and gp34-FcRn interaction.

First, one experiment was performed demonstrating interference between gp34 and immobilized IgG as already shown before, this time testing anti-gp34wt and anti-gp34mtrp mAbs within the same experimental setup. Results obtained reflect previous results.

FIG. 46 shows the IgG blocking ability by anti-gp34-specific mAbs. Anti-gp34wt-specific mAbs (10 μg/ml; 5 μg/ml or 2.5 μg/ml) were pre-incubated with recombinant, sgp34wt (constant amount of 1 μg/ml) and added to IgG-immobilized ELISA plates. Bound sgp34wt-V5-His in absence or presence of blocking mAbs is detected using anti-V5-HRP antibody. Most anti-gp34wt-specific mAbs have the potential to efficiently block vFcγR gp34 IgG-binding but with strong variations in efficiency. In addition, some gp34mtrp mAbs show a tendency for inhibition, while some mAbs do not essentially affect IgG-binding by gp34. Mouse IgG1 and IgG2a isotypes were used for reference and should not specifically influence IgG-interaction. In addition, binding of sgp34wt to immobilized IgG in absence of any mAb is depicted.

Next, sgp34wt and CRP interaction was analyzed, included various anti-gp34 mAbs, and identified their potential to block this interaction. Experiments were performed as described before for IgG-blocking ELISA, but this time using immobilized CRP as gp34-ligand.

Those mAbs exhibiting the strongest blocking effect on IgG-binding did not at all block the interaction towards CRP. In contrast, mAbs raised against the sgp34mtrp variant, impaired for IgG-binding and structurally impeded to form higher oligomers at least block gp34-CRP-binding by ˜50%. Interestingly, also two mAbs raised against sgp34wt (wt.07 and wt.08) have the same blocking capacity as those mAbs. Based on phylogenetic tree analysis this makes sense, since they show closest relationship on protein level of their respective Fab-sequences (data not shown).

Further, this led to the assumption, that different epitopes of gp34wt protein are necessary for binding of vFcγR gp34 to IgG, compared to CRP. Moreover, this is underlined by unpublished data from our group (also shown here in this study): while gp34mtp protein is impaired for IgG-binding it remains binding ability towards CRP, although not to the same extend like gp34wt protein.

FIG. 47 shows the CRP blocking ability by anti-gp34-specific mAbs. Anti-gp34wt-specific mAbs (10 μg/ml; 5 μg/ml or 2.5 μg/ml or without any mAb) were pre-incubated with recombinant, sgp34wt (constant amount of 20 μg/ml) and added to CRP-immobilized ELISA plates. Bound sgp34wt-V5-His in absence or presence of blocking mAbs is detected using anti-V5-HRP antibody. Most anti-gp34mtrp-specific mAbs have the potential to block vFcγR gp34 CRP-binding up to 50% but with strong variations in efficiency. Here, mtrp.05 and mtrp.06 show less efficient blocking ability. Only wt.07 and wt.08 mAbs raised against sgp34wt protein have an effect on CRP-binding while all the other gp34wt mAbs and anti-gp68 mAbs (as control) does not block this interaction. Mouse IgG1 and IgG2a isotype controls were used for reference and should not influence CRP-interaction. In addition, binding of sgp34wt to immobilized CRP in absence of any mAb is depicted.

Furthermore, the gp34wt and FcRn interaction was investigated whereby various anti-gp34 mAbs were analyzed and their potential to block this interaction was identified. Experiments were performed as described before for IgG-blocking ELISA, but this time using immobilized FcRn as a novel gp34-ligand. Interaction of gp34 and FcRn was illustrated earlier.

Those mAbs exhibiting the strongest blocking effect on IgG-binding did not at all block the interaction towards FcRn. In contrast, mAbs raised against the gp34mtrp variant, impaired for IgG-binding and structurally impeded to form higher oligomers at least block gp34-CRP-binding by ˜50%. Interestingly, also two mAbs (wt.07 and wt.08) have the same blocking capacity as those mAbs.

Moreover, those mAbs showing effects on the gp34 interaction with FcRn are also affecting the gp34-CRP interaction. This led us to speculate that those epitopes implicated in the gp34 interaction with CRP might be overlapping with binding sites for gp34 and FcRn interaction but distant from regions necessary for binding of vFcγR gp34 to IgG.

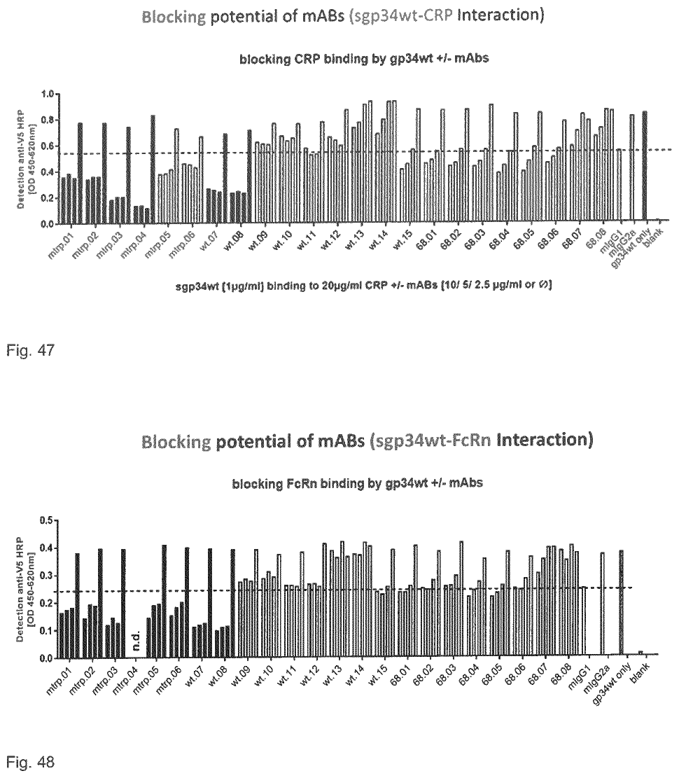

FIG. 48 shows the FcRn blocking ability by anti-gp34-specific mAbs. Anti-gp34wt-specific mAbs (10 μg/ml; 5 μg/ml or 2.5 μg/ml or without any mAb) were pre-incubated with recombinant, sgp34wt (constant amount of 1 μg/ml) and added to FcRn-immobilized ELISA plates. Bound sgp34wt-V5-His in absence or presence of blocking mAbs is detected using anti-V5-HRP antibody. All anti-gp34mtrp-specific mAbs have the potential to block vFcγR gp34 FcRn-binding up to 50% with similar efficiencies. Only wt.07 and wt.08 mAbs raised against sgp34wt protein have an effect on FcRn-binding while all the other anti-gp34wt mAbs and anti-gp68 mAbs (as control) does not block this interaction. Mouse IgG1 and IgG2a isotypes were used for reference and should not influence FcRn-interaction. In addition, binding of sgp34wt to immobilized FcRn in absence of any mAb is depicted.

In conclusion, mAbs raised against HCMV encoded vFcγRs gp34 and gp68 are provided, exhibiting various interesting properties and even more important have the potential to interfere with IgG-binding abilities of vFcγR gp68.

Regarding vFcγR gp34, powerful mAbs are provided interfering with binding partners of gp34 involving IgG, CRP and FcRn.

The properties and blocking potentials of the anti-vFcγR gp34 mAbs are shown in Table 9.

TABLE 9
Summary anti-gp34 mAbs
Summarizing properties of anti-gp34 mAbs
with respect to IgG, CRP and FcRn.
We generated in total 15 anti-gp34-specific
mAbs and original motherwells (MW)
are indicated from hybridomas further
sub-cloned by limiting dilutions. mAbs
anti-gp34mtrp (immunization with sgp34mtrp
protein) and mAbs anti-gp34 wt (immunization
with sgp34 wt protein) were further characterized
in ELISA, WB and precipitation experiments.
Finally, all of them were tested upon their
potential to block gp34 wt CRP-binding and
FcRn-binding resulting in candidates showing
promising potential to interfere with IgG-binding,
CRP-binding and/or FcRn-binding ability of gp34 wt.

| | | | | ELISA | ELISA | WB | WB | IP on | | | |
|---|---|---|---|---|---|---|---|---|---|---|---|
| | MW | subclass | SEQ CDRs | 34 wt | mtrp | 34 wt | mtrp | HCMV | IgG Blocking | CRP Blocking | FcRn Blocking |
| mtrp.01 | 2H3 | mlgG1 | + | +++ | +++ | ++ | ++ | + | + | + | + |
| mtrp.02 | 2H3 | mlgG1 | + | +++ | +++ | ++ | ++ | + | + | + | + |
| mtrp.03 | 3D11 | mlgG1 | + | +++ | +++ | ++ | ++ | ++ | ++ | ++ | + |
| mtrp.04 | 3D11 | mlgG1 | + | +++ | +++ | ++ | ++ | + | ++ | ++ | + |
| mtrp.05 | AE8 | mlgG1 | + | +++ | +++ | + | (+) | + | (+) | + | + |
| mtrp.06 | AE8 | mlgG1 | + | +++ | +++ | + | (+) | + | (+) | + | + |
| wt. 07 | 4B4 | mlgG1 | + | +++ | − | ++ | − | n.d. | (+) | − | + |
| wt. 08 | 4B4 | mlgG1 | + | +++ | − | ++ | − | n.d. | (+) | − | + |
| wt. 09 | 4H7 | mlgG1 | + | +++ | − | ++ | − | n.d. | +++ | − | − |
| wt. 10 | 4H7 | mlgG1 | + | +++ | − | ++ | − | n.d. | +++ | − | − |
| wt. 11 | SA11 | mlgG1 | + | +++ | − | ++ | − | n.d. | +++ | − | − |
| wt. 12 | SA11 | mlgG1 | + | +++ | − | ++ | − | n.d. | +++ | − | − |
| wt. 13 | 6E3 | mlgG2a | + | + | − | ++ | − | n.d. | − | − | − |
| wt. 14 | 6E3 | mlgG2a | + | + | − | ++ | − | n.d. | − | − | − |
| wt. 15 | 3F5 | mlgG1 | + | +++ | + | − | + | n.d. | +++ | − | − |

### Potential to Use Anti-vFcγR mAbs for Diagnostic Applications

Both vFcγRs gp34 and gp68 are present in the HCMV virion. Therefore, it was investigated whether vFcγR gp34 can be detected as indicator for HCMV virions. These experiments were carried out using anti-gp34 mAbs according to the present invention.

The data indicate a potential for usage of anti-vFcγR mAbs for diagnostic applications. Since vFcγR gp34 represents a glycoprotein binding to human IgG, human IgG-Fc-fragment was immobilized before incubated with sera of various patient samples.

Presence of gp34 was verified using anti-gp34 mAb mtrp.04 for detection. HCMV virions were further determined using commercial anti-gB antibody. Glycoprotein B is one important surface protein of HCMV, present also within the virion.

The data indicate a potential to use anti-gp34 mAbs for detection of HCMV positive sera. Data will be further analyzed since not all HCMV-positive sera were also positive for vFcγR gp34 indicating higher gp34 levels under different circumstances e.g. re-activation of the virus. All tested sera from diagnosed HCMV-negative patients were also negative in our ELISAs using mAb anti-gp34 for detection.

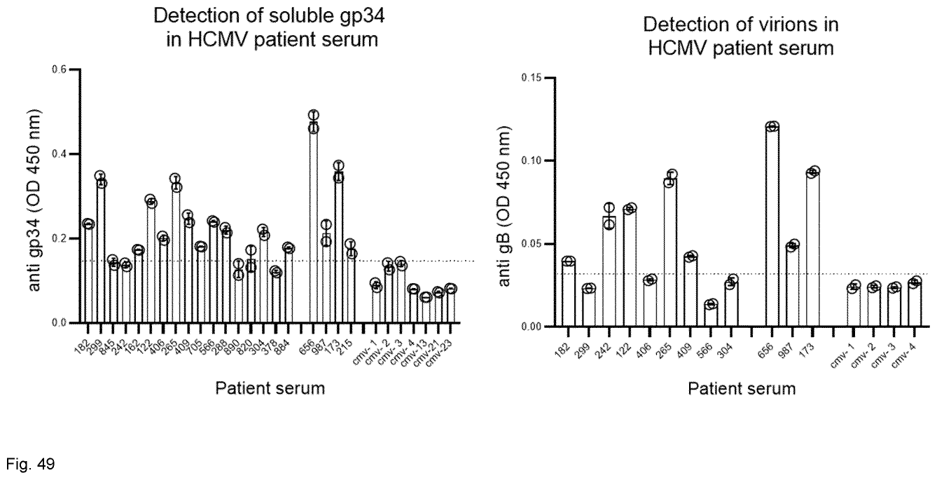

FIG. 49: Detection of vFcγRs gp34 by anti-gp34mtrp-specific mAb mtrp.04 in various patient sera. Ultra-centrifuged (at 100,000 g for 45 minutes at 4° C.) sera from various donors were investigated for their HCMV-sero-positivity by usage of either commercially available anti-HCMV gB antibody or using our anti-gp34 mAb mtrp.04. Respective secondary antibody (goat-anti-mouse IgG HRP) was added to detect any gp34 or gB present in each serum indicating HCMV seropositive donors. Sera from HCMV-negative donors served as control.

Next, the data were validated further and used different sera from HCMV donors after primary infection and sera from donors after re-activation or re-infection. Again, sera from HCMV negative patients were used as negative control. This time, ELISA was performed using only anti-gp34 mAb for detection. Not all, but many sera correlated with gp34 expression and sero-positivity. This needs to be further validated and viral load within these sera has to be measured to validate if it correlates with gp34 levels.

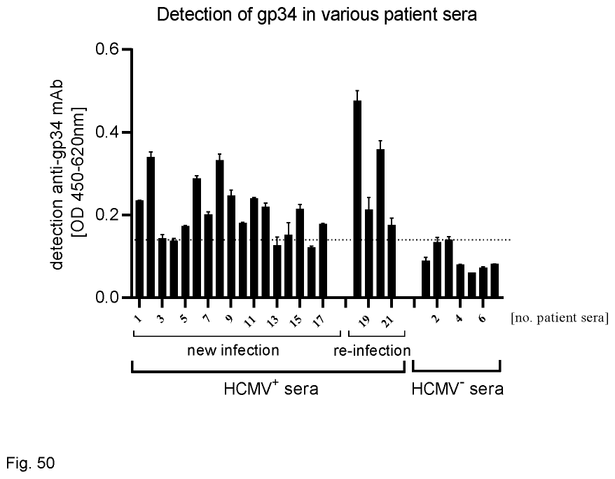

FIG. 50: Detection of vFcγRs gp34 by anti-gp34-specific mAb mtrp.04 in HCMV+ vs. HCMV− patient sera. Ultra-centrifuged (at 100,000 g for 45 minutes at 4° C.) sera from HCMV+ or HCMV− donors were incubated on an IgG-Fc coated (4 μg/ml) ELISA plate overnight at 4 degrees. Only supernatants after ultracentrifugation were analyzed, while the sediment should contain virions, exosomes and cell membranes.

The plate was blocked and incubated with antigp34mtrp mAb mtrp.04 at room temperature for 4 hrs. A secondary antibody (goat-anti-mouse IgG-HRP) was used to detect any gp34 present in each serum by spectrophotometry.

### Recombinant Fab Cloning

For further analysis Fab fragments were provided based on the methodology as known to the skilled person.

In brief, hybridomas of mAbs were cultivated. RNA was isolated and transcribed into cDNA. This cDNA was used for amplification of either variable light chain or variable heavy chain und further ligated into plasmids containing either mouse IgG1 heavy chain with an intact Poly-His-Tag (pOPINVH plasmid for variable heavy chain) at the C-terminus or mouse IgG kappa light chain (pOPINVL plasmid for variable light chain). Within the light chain plasmid, a stop codon is introduced, resulting in a frame shift leading to a non-functional poly-His-Tag. Therefore, only functional heavy chain fab-fragments expresses a poly-His-Tag. In addition, heavy chains only stabilized and secreted into cell culture supernatants if connected to their respective light chains.

Recombinant Fab-fragments of anti-vFcγR gp34 and anti-gp68 mAbs were cloned and verified (i) for their secretion into supernatants after double transfection of pOPINVL- and pOPINVH-plasmids in 293-T cells detected by anti-His, (ii) their specific recognition of their appropriate antigen (comparison with full length mAbs) and (iii) recombinant fab-fragments were send for sanger-sequencing. By help of sequencing and online tools (e.g. IgBLAST from NCBI) nucleotides and amino acids of framework regions (FR1-4) and complementarity-determining regions (CDRs 1-3) of each individual mAb were determined. Sequences were applied for phylogenetic tree analysis using Genious to identify relationship and similarities of all generated anti-vFcγR mAbs (data not shown).

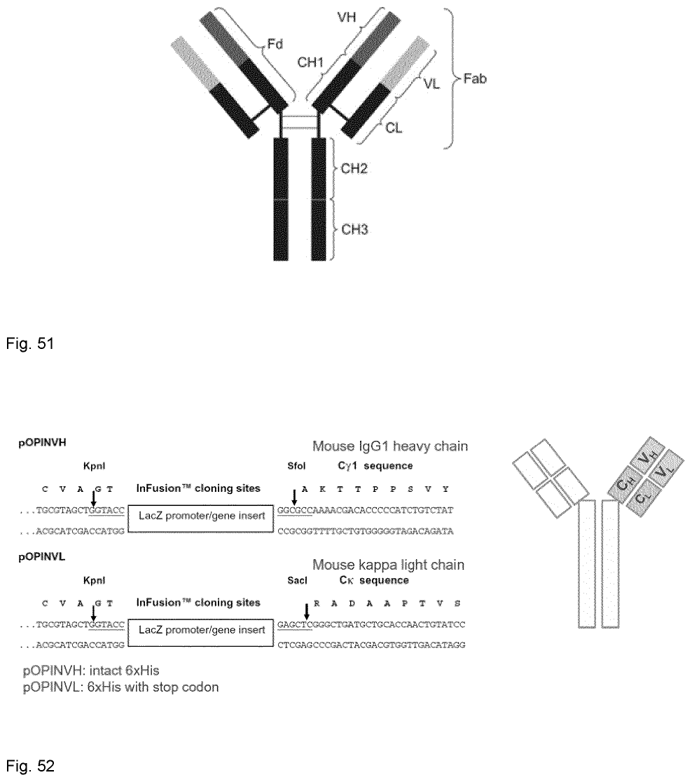

FIG. 51 is a schematic representation of human IgG1. Human immunoglobulin 1 subclass is illustrated and Fab regions are indicated comprising VH (variable heavy chain), VL (variable light chain). VH and VL of respective mAbs were amplified by PCR from hybridoma cDNA. Sequences were generated by in-Fusion cloning and inserted into pOPINVL or pOPINVH plasmids, containing in-frame mouse IgG1 heavy chain or mouse IgG kappa light chain, respectively.

FIG. 52: In-fusion cloning using pOPINVL and pOPINVH plasmids. Detailed sequencing informations of pOPINVL or pOPINVH plasmids are depicted. Those include informations like restriction enzymes used for in-fusion ligation and in-frame added elements for both plasmids: either mouse IgG1 heavy chain (pOPINVH, with intact poly-His-Tag) or mouse IgG kappa light chain (pOPINVL, with non-functional poly-His-Tag due to stop mutation introduced into the plasmids in frame in front of the original poly-His-Tag).

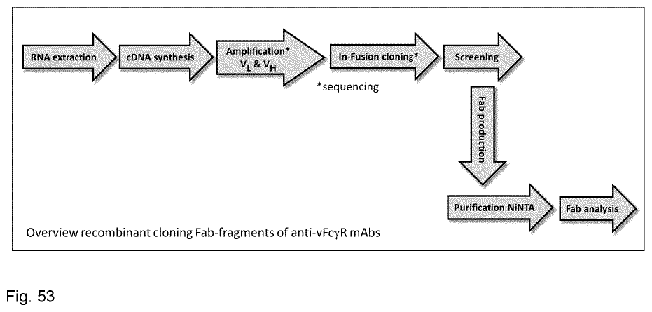

FIG. 53: Workflow generation recombinant fab-fragments of anti-vFcγR mAbs. Essential steps in cloning Fab-fragments (Fabs) of anti-vFcγR mAbs are shown, including RNA isolation from hybridoma cell culture; PCR-amplification of VL and VH fragments using hybridoma cDNAs. Constructs were cloned by in-fusion technology. Correct and functional Fab-fragments were tested for antigen-recognition and fab-expression after co-transfection of VL and VH plasmids into HEK-293-T cells. Appropriate amounts of cell culture supernatants were collected under starvation conditions (no FCS within the media) and sub following purified using NiNTA chromatography. Fab fragment were further analyzed by sequencing and functional studies.

Within a first experiment, a selection of newly generated Fab-fragments was analyzed if they remain their blocking capacity regarding IgG binding by vFcγR gp34.

Binding of sgp34wt to immobilized IgG was investigated in the presence or absence of blocking candidate. Within the experiment, full length mAbs were compared to their respective recombinant, purified Fab-fragment.

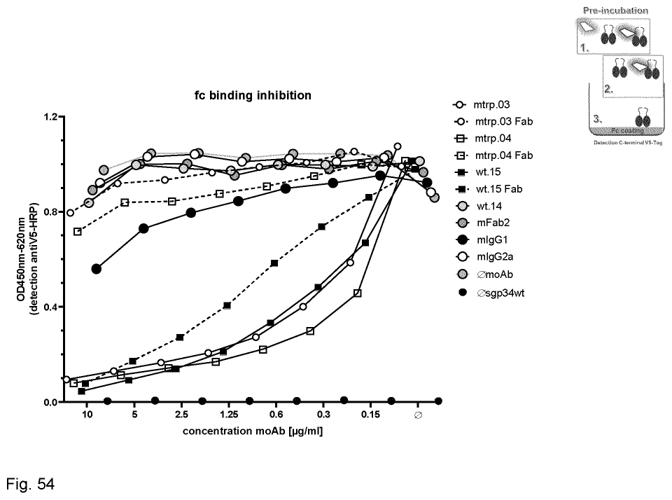

FIG. 54: IgG blocking ability by anti-gp34 mAbs. Anti-gp34-specific mAbs (members of both groups) or cloned recombinant Fab-fragments thereof were pre-incubated with recombinant, soluble sgp34wt (constant amount 1ug/ml) and added to IgG-immobilized ELISA plates. Bound sgp34wt in absence or presence of blocking full-length mAbs or fab-fragments only is detected using anti-V5-HRP antibody recognizing IgG-bound sgp34wt-V5-His. Most tested anti-gp34wt-specific full length mAbs (except wt.13 and wt.14) have the potential to block vFcγR gp34 IgG-binding and for the Fab-fragment of anti-gp34wt mAb wt.15 blocking capacity remains although with a bit reduced efficiency. Whereas, blocking capacity by the fab-fragment derived from anti-gp34mtrp mAbs mtrp.03 and mtrp.04, which were sufficient before to block gp34wt-IgG binding as full length mAb, is gone. Dose-dependent IgG-blocking potential is indicated. Also, non-specific effect of total mouse IgG1, as well as mouse IgG1 fab-fragment isotype control is shown. In addition, binding of sgp34wt to immobilized IgG in absence of any full-length mAb or fab-fragment is depicted.

The first experiment verified that anti-gp34 mAb wt.15 still remains its blocking ability regarding the sgp34wt-IgG interaction, although it lost a bit of its potential. In contrast, mtrp.03 and mtrp.04 mAb as Fab only fragments completely seemed to lack their blocking capacity. This result indicates that blocking effects seen before in our ELISAs were maybe due to the Fc-part. Nevertheless, experiments need to be repeated and results further analyzed in more detail. Also, mtrp mAbs, compared to some of the sgp34wt mAbs never had such a drastically blocking effect up to nearly no sgp34wt-Fc-binding in presence of those mAbs.

By cloning all respective Fab-fragments further analysis can be done. In addition, by sequencing our plasmids containing variable heavy and light chain fragments of our mAbs, we were able to determine their sequences and predict their framework regions (FR) and complementary-determining-regions (CDRs), both are essential for humanization of mAbs. Following, one representative example is shown, illustrating sequence analysis (using GeneWiz) and predictions of variable regions (using IgBlast).

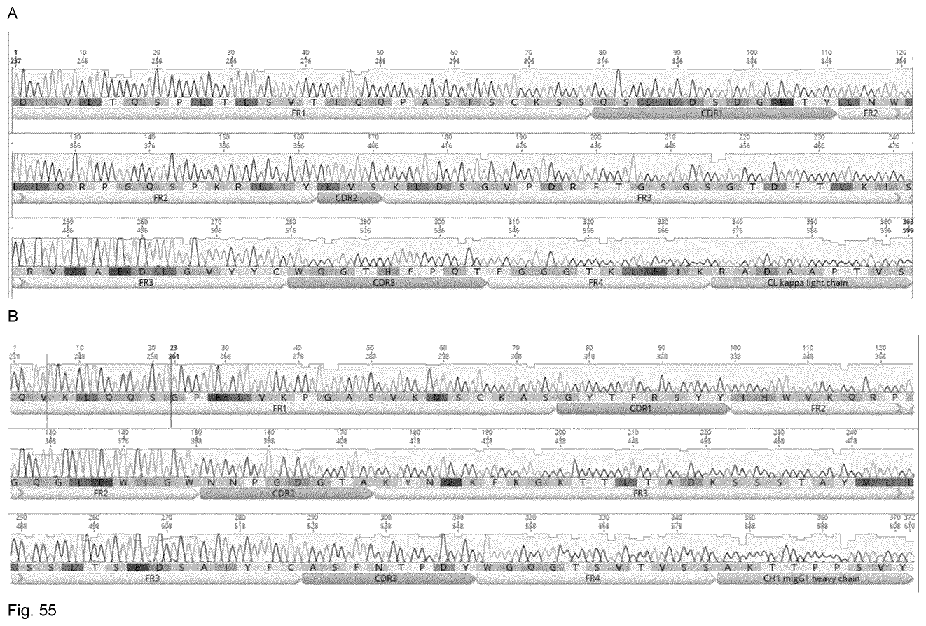

FIG. 55 shows representative variable light chain (VL) and variable heavy chain (VH) of anti-gp34 wt.15. Fab-fragment consist of one variable light chain (A) and one variable heavy chain (B). Variable light chain of hybridoma gp34wt.15 was cloned and send for sequencing (FIG. 52A). Obtained sequences were analyzed using Geneious software and IgBlast. Framework-regions (FR) as well as complementarity-determining-regions (CDRs) are indicated. Also, mouse IgG kappa light chain is indicated, contained in-frame on pOPINVL plasmids. (B) Variable heavy chain of hybridoma gp34wt.15 was cloned and send for sequencing. Obtained sequences were analyzed using Geneious software and IgBlast. Framework-regions (FR) as well as complementarity-determining-regions are indicated. Also, mouse IgG1 heavy chain is indicated, contained in-frame on pOPINVH plasmids. Also, heavy chain fragments contain a function poly-His-Tag on the C-terminus, used for detection and purification approaches.

Since all anti-vFcγR mAbs were successfully cloned, sequences were available and were analyzed e.g. by phylogenetic tree analysis comparing protein sequences to decipher relationships (data not shown).

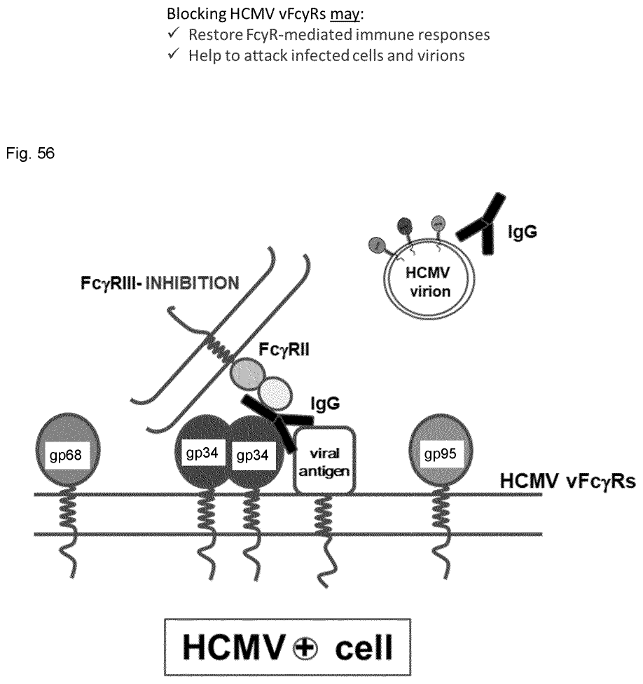

FIG. 56 shows schematically the immune evasion mechanism performed by HCMV encoded vFcγRs. HCMV-encoded vFcγRs like gp34 and gp68 expressed during infection mediate immune evasion from IgG-triggered immune responses by antagonizing host FcγR-activation. This is enabled by their property to bind human IgG-Fc thereby counteracting simultaneously interactions of host FcγRs. At least, vFcγRs gp34 and gp68 are also part of the HCMV virion, potentially protecting the virion from Antibody-mediated immune-recognition.

The amino acid sequences of the preferred monoclonal antibodies are provided as follows:

Sequences including CRDs of monoclonal anti-gp68 and anti-gp34 antibodies:
1) Anti gp34 clone mtrp.01
Heavy chain variable region polypeptide sequence, 145 amino acids
FR1-CDR1-FR2-CDR2-FR3-CDR3-FR4
SEQ mtrp.01 HC1_.ab1:
(SEQ ID NO: 14)
MGILPSPGMPALLSLVSLLSVLLMGCVAQVKLEESGAELVKPGASVKLSCTGSGFNIKDIYM
DWVKQRPEQGLEWIGRIDPANGNSKYDPKFQGKATITVDTSSNTAYLQLSSLTSEDTAVYY
CASYYGSSYNYWGQGTTLTVSS
Light chain variable region polypeptide sequence, 149 amino acids
FR1-CDR1-FR2-CDR2-FR3-CDR3-FR4
SEQ mtrp.01 LC15_.ab1:
(SEQ ID NO: 15)
MGILPSPGMPALLSLVSLLSVLLMGCVADIVITQTPPSVPVTPGESVSISCRSSKSLLHSNGN
TYLYWFLQRPGQSPQLLIYRMSNLASGVPDRFSGSGSGTAFTLRISRVEAEDVGVYYCMQ
HLEYPLTFGAGTKLELKRADAAPTVS
2) Anti gp34 clone mtrp.02
Heavy chain variable region polypeptide sequence, 145 amino acids
FR1-CDR1-FR2-CDR2-FR3-CDR3-FR4
SEQ mtrp.02 HC3_.ab1:
(SEQ ID NO: 16)
MGILPSPGMPALLSLVSLLSVLLMGCVAQVKLEQSGAELVKPGASVKLSCTGSGFNIKDIYM
DWVKQRPEQGLEWIGRIDPANGNSKYDPKFQGKATITVDTSSNTAYLQLSSLTSEDTAVYY
CASYYGSSYNYWGQGTTLTVSS
Light chain variable region polypeptide sequence, 149 amino acids
FR1-CDR1-FR2-CDR2-FR3-CDR3-FR4
SEQ mtrp.02 LC5_.ab1:
(SEQ ID NO: 17)
MGILPSPGMPALLSLVSLLSVLLMGCVADIVITQTPPSVPVTPGESVSISCRSSKSLLHSNGN
TYLYWFLQRPGQSPQLLIYRMSNLASGVPDRFSGSGSGTAFTLRISRVEAEDVGVYYCMQ
HLEYPLTFGAGTKLELKRADAAPTVS
3) Anti gp34 clone mtrp.03
Heavy chain variable region polypeptide sequence, 143 amino acids
FR1-CDR1-FR2-CDR2-FR3-CDR3-FR4
SEQ mtrp.03 HC1_.ab1:
(SEQ ID NO: 18)
MGILPSPGMPALLSLVSLLSVLLMGCVAEVQLEESGTVLARPGASVKMSCKASGYTFTSYW
MHWVKQRPGQGLEWIGAIYPGNSDTGYNQKFKGKARLTAVTSTSTAYLELSSLTDEDSAV
YFCTRLGYGDYWGQGTTLTVSS
Light chain variable region polypeptide sequence, 135 amino acids
SEQ_mtrp.03 LC1_.ab1:
FR1-CDR1-FR2-CDR2-FR3-CDR3-FR4
(SEQ ID NO: 19)
MGILPSPGMPALLSLVSLLSVLLMGCVADIVLTQTPSSMYASLGERVTITCKASQDINSYLTW
FQQKPGKSPKTLIYRANRLVDGVPSRFSGSGSGQDYSLTINSLDYEDMGIYYCLQYDEFPP
TFGGGTKLEIK
4) Anti gp34 clone mtrp.04
Heavy chain variable region polypeptide sequence, 143 amino acids
FR1-CDR1-FR2-CDR2-FR3-CDR3-FR4
SEQ mtrp.04_HC1_.ab1:
(SEQ ID NO: 20)
MGILPSPGMPALLSLVSLLSVLLMGCVAEVKLVQSGTVLARPGASVKMSCKASGYTFTSYW
MHWVKQRPGQGLEWIGAIYPGNSDTGYNQKFKGKARLTAVTSTSTAYLELSSLTDEDSAV
YFCTRLGYGDYWGQGTTLTVSS
Light chain variable region polypeptide sequence, 135 amino acids
SEQ_mtrp.04 LC3_.ab1:
FR1-CDR1-FR2-CDR2-FR3-CDR3-FR4
(SEQ ID NO: 21)
MGILPSPGMPALLSLVSLLSVLLMGCVADIVMTQTPSSMYASLGERVTITCKASQDINSYLT
WFQQKPGKSPKTLIYRANRLVDGVPSRFSGSGSGQDYSLTINSLDYEDMGIYYCLQYDEFP
PTFGGGTKLEIK
5) Anti gp34 clone mtrp.05
Heavy chain variable region polypeptide sequence, 145 amino acids
FR1-CDR1-FR2-CDR2-FR3-CDR3-FR4
SEQ mtrp.05_HC3_.ab1:
(SEQ ID NO: 22)
MGILPSPGMPALLSLVSLLSVLLMGCVAEVKLLQSGPEVKKPGETVKISCKASGYTFTNYGM
SWLRQAPGKGLKWMGWINTYTGEPTYADDFKGRFAFSLETSASTAYFQINNLKDEDTTTYF
CARRAFGGLDYWGQGTSVTVSS
Light chain variable region polypeptide sequence, 140 amino acids
SEQ_mtrp.05 LC4_.ab1:
FR1-CDR1-FR2-CDR2-FR3-CDR3-FR4
(SEQ ID NO: 23)
MGILPSPGMPALLSLVSLLSVLLMGCVADIVMTQSPLSLPVSLGDQASISCRSSQSIVYTNG
NTYLEWYLQKPGQSPKLLIYKVSNRFSGVPDRFSGSGSGTDFTLKISRVEAEDLGVYYCFQ
GSHVPWTFGGGTKLEIK
6) Anti gp34 clone mtrp.06
Heavy chain variable region polypeptide sequence, 145 amino acids
FR1-CDR1-FR2-CDR2-FR3-CDR3-FR4
SEQ mtrp.06_HC1_.ab1:
(SEQ ID NO: 24)
MGILPSPGMPALLSLVSLLSVLLMGCVAEVQLLQSGPEVKKPGETVKISCKASGYTFTNYG
MSWLRQAPGKGLKWMGWINTYTGEPTYADDFKGRFAFSLETSASTAYFQINNLKNEDTTT
YFCARRAFGGLDYWGQGTSVTVSS
Light chain variable region polypeptide sequence, 140 amino acids
SEQ_mtrp.06 LC1_.ab1:
FR1-CDR1-FR2-CDR2-FR3-CDR3-FR4
(SEQ ID NO: 25)
MGILPSPGMPALLSLVSLLSVLLMGCVADIVLTQSPLSLPVSLGDQASISCRSSQSIVYTNGN
TYLEWYLQKPGQSPKLLIYKVSNRFSGVPDRFSGSGSGTDFTLKISRVEAEDLGVYYCFQG
SHVPWTFGGGTKLEIK
7) Anti gp34 clone wt.07
Heavy chain variable region polypeptide sequence, 149 amino acids
FR1-CDR1-FR2-CDR2-FR3-CDR3-FR4
SEQ wt.07 HC2_.ab1:
(SEQ ID NO: 26)
MGILPSPGMPALLSLVSLLSVLLMGCVAQVKLEESGPELVKTGASVKISCKASGYSFTSYQI
YWVKQSHGKSLEWIGHISCYNGATSYNQKFKDKATFTVDTSSSTAYMQFNSLTSEDSAVY
FCARYDYAPDYYAMDYWGQGTSVTVSS
Light chain variable region polypeptide sequence, 139 amino acids
SEQ_wt.07 LC_.ab1:
FR1-CDR1-FR2-CDR2-FR3-CDR3-FR4
(SEQ ID NO: 27)
MGILPSPGMPALLSLVSLLSVLLMGCVADIVLTQTPASLAVSLGQRATISCRASESVDSYGN
SFMHWYQQKPGQPPKLLIYLASNLESGVPARFSGSGSRTDFTLTIDPVEAEDAAIYYCQQN
NEDPLTFGGGTKLELK
8) Anti gp34 clone wt.08
Heavy chain variable region polypeptide sequence, 149 amino acids
FR1-CDR1-FR2-CDR2-FR3-CDR3-FR4
SEQ wt.08 HC3_.ab1:
(SEQ ID NO: 28)
MGILPSPGMPALLSLVSLLSVLLMGCVAEVQLEQSGPELVKTGASVKISCKASGYSFTSYQI
YWVKQSHGKSLEWIGHISCYNGATSYNQKFKDKATFTVDTSSSTAYMQFNSLTSEDSAVY
FCARYDYAPDYYAMDYWGQGTSVTVSS
Light chain variable region polypeptide sequence, 139 amino acids
SEQ_wt.08 LC3_.ab1:
FR1-CDR1-FR2-CDR2-FR3-CDR3-FR4
(SEQ ID NO: 29)
MGILPSPGMPALLSLVSLLSVLLMGCVADIVMTQTPASLAVSLGQRATISCRASESVDSYGN
SFMHWYQQKPGQPPKLLIYLASNLESGVPARFSGSGSRTDFTLTIDPVEAEDAAIYYCQQN
NEDPLTFGGGTKLELK
9) Anti gp34 clone wt.09
Heavy chain variable region polypeptide sequence, 143 amino acids
FR1-CDR1-FR2-CDR2-FR3-CDR3-FR4
SEQ wt.09 HC4_.ab1:
(SEQ ID NO: 30)
MGILPSPGMPALLSLVSLLSVLLMGCVAEVQLEQSGPELVKPGASVKMSCKASGYTFTRPYI
HWVKQRPGQGLEWIGWIYPGDGITKYNEKFKGKITLTADKSSSAAYMLLSSLTSEDSAIYFC
ASFTTPVYWGQGTSVTVSS
Light chain variable region polypeptide sequence, 140 amino acids
SEQ_wt.09 LC4_.ab1:
FR1-CDR1-FR2-CDR2-FR3-CDR3-FR4
(SEQ ID NO: 31)
MGILPSPGMPALLSLVSLLSVLLMGCVADIVMTQTPLTLSVTIGQPASISCKSSQSLLDSDGK
TYLNWLFQRPGQSPKRLIYLVSKLDSGVPDRFTGSGSGTDFTLKISRVEAEDLGVYYCWQG
THFPQTFGGGTKLEIK
10) Anti gp34 clone wt.10
Heavy chain variable region polypeptide sequence, 143 amino acids
FR1-CDR1-FR2-CDR2-FR3-CDR3-FR4
SEQ wt.10 HC1_.ab1:
(SEQ ID NO: 32)
MGILPSPGMPALLSLVSLLSVLLMGCVAEVKLEQSGPELVKPGASVKMSCKASGYTFTRPYI
HWVKQRPGQGLEWIGWIYPGDGITKYNEKFKGKITLTADKSSSAAYMLLSSLTSEDSAIYFC
ASFTTPVYWGQGTSVTVSS
Light chain variable region polypeptide sequence, 140 amino acids
SEQ_wt.10 LC1_.ab1:
FR1-CDR1-FR2-CDR2-FR3-CDR3-FR4
(SEQ ID NO: 33)
MGILPSPGMPALLSLVSLLSVLLMGCVADIVLTQTPLTLSVTIGQPASISCESSQSLLDSDGK
TYLNWLFQRPGQSPKRLIYLVSKLDSGVPDRFTGSGSGTDFTLKICRVEAEDLGVYYCWQ
GTHFPQTFGGGTKLEIK
11) Anti gp34 clone wt.11
Heavy chain variable region polypeptide sequence, 143 amino acids
FR1-CDR1-FR2-CDR2-FR3-CDR3-FR4
SEQ wt.11 HC1_.ab1:
(SEQ ID NO: 34)
MGILPSPGMPALLSLVSLLSVLLMGCVAQVQLEESGPELVKPGASVKMSCKASGYTVTRSY
IHWVKQRPGQGLEWIGWIYPGDGSTKYNEKFKGKTTLTVDKSSSTAYMLLSSLTSEDSAIYF
CASFTTPVYWGQGTSVTVSS
Light chain variable region polypeptide sequence, 140 amino acids
SEQ_wt.11 LC1_.ab1:
FR1-CDR1-FR2-CDR2-FR3-CDR3-FR4
(SEQ ID NO: 35)
MGILPSPGMPALLSLVSLLSVLLMGCVADIVLTQSPLTLSVTIGQPASISCKSSQSLLDSDGE
TYLNWLLQRPGQSPKRLIYLVSKLDSGVPDRFTGSGSGTDFTLKISRVEAEDLGVYYCWQG
THEPQTFGGGTKLEIK
12) Anti gp34 clone wt.12
Heavy chain variable region polypeptide sequence, 143 amino acids
FR1-CDR1-FR2-CDR2-FR3-CDR3-FR4
SEQ wt.12 HC4_.ab1:
(SEQ ID NO: 36)
MGILPSPGMPALLSLVSLLSVLLMGCVAEVKLQESGPELVKPGASVKMSCKASGYTVTRSYI
HWVKQRPGQGLEWIGWIYPGDGSTKYNEKFKGKTTLTVDKSSSTAYMLLSSLTSEDSAIYF
CASFTTPVYWGQGTSVTVSS
Light chain variable region polypeptide sequence, 140 amino acids
SEQ_wt.12 LC1_.ab1:
FR-CDR1-FR2-CDR2-FR3-CDR3-FR4
(SEQ ID NO: 37)
MGILPSPGMPALLSLVSLLSVLLMGCVADIVLTQTPLTLSVTIGQPASISCKSSQSLLDSDGE
TYLNWLLQRPGQSPKRLIYLVSKLDSGVPDRFTGSGSGTDFTLKISRVEAEDLGVYYCWQG
THFPQTFGGGTKLEIK
13) Anti gp34 clone wt.13
Heavy chain variable region polypeptide sequence, 145 amino acids
FR1-CDR1-FR2-CDR2-FR3-CDR3-FR4
SEQ wt.13 HC2_.ab1:
(SEQ ID NO: 38)
MGILPSPGMPALLSLVSLLSVLLMGCVAEVOLVESGPELKKPGETVKISCKASGYAFTNYG
MNWVKQTPGKGLKWMGWINTYTGKPTYADDFKGRFAFSLETSASSAYLQINNLKNEDTAT
YFCARRRGNSFDYWGQGTTLTVSS
Light chain variable region polypeptide sequence, 141 amino acids
SEQ_wt.13 LC4_.ab1:
FR1-CDR1-FR2-CDR2-FR3-CDR3-FR4
(SEQ ID NO: 39)
MGILPSPGMPALLSLVSLLSVLLMGCVADIVLTQSPSSLTVTAGEKVTMSCKSSQSLLNSGN
QENCLTWYQQKPGQPPRLLIYWASTRESGVPDRFTGSGSGTDFTLTISSVQAEDLAVYYC
QNDYTYPLTFGAGTKLELK
14) Anti gp34 clone wt.14
Heavy chain variable region polypeptide sequence, 145 amino acids
FR1-CDR1-FR2-CDR2-FR3-CDR3-FR4
SEQ wt.14 HC4_.ab1:
(SEQ ID NO: 40)
MGILPSPGMPALLSLVSLLSVLLMGCVAQVKLLQSGPELKKPGETVKISCKASGYAFTNYG
MNWVKQTPGKGLKWMGWINTYTGKPTYADDFKGRFAFSLETSASSAYLQINNLKNEDTAT
YFCARRRGNSFDYWGQGTTLTVSS
Light chain variable region polypeptide sequence, 141 amino acids
SEQ_wt.14 LC1_.ab1:
FR-CDR1-FR2-CDR2-FR3-CDR3-FR4
(SEQ ID NO: 41)
MGILPSPGMPALLSLVSLLSVLLMGCVADIVITQSPSSLTVTAGEKVTMSCKSSQSLLNSGN
QENCLTWYQQKPGQPPRLLIYWASTRESGVPDRFTGSGSGTDFTLTISSVQAEDLAVYYC
QNDYTYPLTFGAGTKLELK
15) Anti gp34 clone wt.15
Heavy chain variable region polypeptide sequence, 143 amino acids
FR1-CDR1-FR2-CDR2-FR3-CDR3-FR4
SEQ wt.15 HC1_.ab1:
(SEQ ID NO: 42)
MGILPSPGMPALLSLVSLLSVLLMGCVAQVKLQQSGPELVKPGASVKMSCKASGYTFRSYY
IHWVKQRPGQGLEWIGWNNPGDGTAKYNEKFKGKTTLTADKSSSTAYMLLSSLTSEDSAIY
FCASFNTPDYWGQGTSVTVSS
Light chain variable region polypeptide sequence, 140 amino acids
SEQ_wt.15 LC1_.ab1:
FR1-CDR1-FR2-CDR2-FR3-CDR3-FR4
(SEQ ID NO: 43)
MGILPSPGMPALLSLVSLLSVLLMGCVADIVLTQSPLTLSVTIGQPASISCKSSQSLLDSDGE
TYLNWLLQRPGQSPKRLIYLVSKLDSGVPDRFTGSGSGTDFTLKISRVEAEDLGVYYCWQG
THFPQTFGGGTKLEIK
16) Anti gp68 clone 68.01
Heavy chain variable region polypeptide sequence, 146 amino acids
FR1-CDR1-FR2-CDR2-FR3-CDR3-FR4
SEQ 68.01 HC2_.ab1:
(SEQ ID NO: 44)
MGILPSPGMPALLSLVSLLSVLLMGCVAEVKLLESGPELVKPGSSVKISCKASGYSFADHIIL
WVKQSHGKSLEWIGNINPYSGNTIYNLNFKGKATLTVDKSSSTAYMQLNSLTSEDSAVYYC
ARIEGYYSMDYWGQGTSVTVSS
Light chain variable region polypeptide sequence, 140 amino acids
SEQ_68.01 LC4_.ab1:
FR1-CDR1-FR2-CDR2-FR3-CDR3-FR4
(SEQ ID NO: 45)
MGILPSPGMPALLSLVSLLSVLLMGCVADIVMTQTPLSLPVSLGDQASISCRSSQSIVHSNG
NTYLEWYLQKPGQSPKVLIYKVSKRFSGVPDRFSGSGSGTDFTLKISRVEAEDMGVYYCFQ
GSHVPLTFGAGTKLELK
17) Anti gp68 clone 68.02
Heavy chain variable region polypeptide sequence, 146 amino acids
FR1-CDR1-FR2-CDR2-FR3-CDR3-FR4
SEQ 68.02 HC2_.ab1:
(SEQ ID NO: 46)
MGILPSPGMPALLSLVSLLSVLLMGCVAEVKLLQSGPELVKPGSSVKISCKASGYSFADHIIL
WVKQSHGKSLEWIGNINPYSGNTIYNLNFKGKATLTVDKSSSTAYMQLNSLTSEDSAVYYC
ARIEGYYSMDYWGQGTSVTVSS
Light chain variable region polypeptide sequence, 140 amino acids
SEQ_68.02 LC2_.ab1:
FR1-CDR1-FR2-CDR2-FR3-CDR3-FR4
(SEQ ID NO: 47)
MGILPSPGMPALLSLVSLLSVLLMGCVADIVLTQTPLSLPVSLGDQASISCRSSQSIVHSNGN
TYLEWYLQKPGQSPKVLIYKVSKRFSGVPDRFSGSGSGTDFTLKISRVEAEDMGVYYCFQ
GSHVPLTFGAGTKLELK
18) Anti gp68 clone 68.03
Heavy chain variable region polypeptide sequence, 146 amino acids
FR1-CDR1-FR2-CDR2-FR3-CDR3-FR4
SEQ 68.03 HC2_.ab1:
(SEQ ID NO: 48)
MGCVAEVKLEESGPEVKKPGETVKISCKASGYTFTNYGMSWLRQAPGKGLKWMGWINTY
TGEPTYADDFKGRFAFSLETSASTAYFQINNLKDEDTTTYFCARRAFGGLDYWGQGTSVT
VFSAKTTPPSVY
Light chain variable region polypeptide sequence, 149 amino acids
SEQ_68.03 LC8_.ab1:
FR1-CDR1-FR2-CDR2-FR3-CDR3-FR4
(SEQ ID NO: 49)
MGILPSPGMPALLSLVSLLSVLLMGCVADIVLTQTPLSLPVSLGDQASISCRSSQSIVHSNGN
TYLEWYLQKPGQSPKVLIYKVSKRFSGVPDRFSGSGSETDFTLKISRVEAEDMGVYYCFQG
SHVPLTFGAGTKLELKRADAAPTVS
19) Anti gp68 clone 68.04
Heavy chain variable region polypeptide sequence, 154 amino acids
FR1-CDR1-FR2-CDR2-FR3-CDR3-FR4
SEQ 68.04 HC4 1/3_.ab1:
(SEQ ID NO: 50)
MGILPSPGMPALLSLVSLLSVLLMGCVAEVQLQESGPEVKKPGETVKISCKASGYTFTNYG
MSWLRQAPGKGLKWMGWINTYTGEPTYADDFKGRFAFSLETSASTAYFQINNLKNEDTTT
YFCARRAFGGLDYWGQGTSVTVSSAKTTPPSVY
Light chain variable region polypeptide sequence, 146 amino acids
FR1-CDR1-FR2-CDR2-FR3-CDR3-FR4
SEQ 68.04 LC3_.ab1:
(SEQ ID NO: 51)
MGILPSPGMPALLSLVSLLSVLLMGCVADIVLTQTPLSLPVSLGDQASISCRSSQNIVHSNGN
TYLEWYLQKPGQSPKLLIYKVSNRFSGVPDRFSGSGSGTDFTLKISRVEAEDLGVYYCFQG
SHVPYTFGGGTKLEVKRADAAPTVS
20) Anti gp68 clone 68.05
Heavy chain variable region polypeptide sequence, 158 amino acids
FR1-CDR1-FR2-CDR2-FR3-CDR3-FR4
SEQ 68.05 HC8_.ab1:
(SEQ ID NO: 52)
MGILPSPGMPALLSLVSLLSVLLMGCVAQVKLEESGAELMKPGASVKISCTATGYTFSGYWI
EWVKQRPGHGLEWIGEILPGSGGTNYNEKFKGKATFTADTSSNTAYMQLSSLTSEDSAVY
YCARGIHYFGYYYFDYWGQGTTLTVSSAKTTPPSVY
Light chain variable region polypeptide sequence, 144 amino acids
FR1-CDR1-FR2-CDR2-FR3-CDR3-FR4
SEQ 68.05 LC1_.ab1:
(SEQ ID NO: 53)
MGILPSPGMPALLSLVSLLSVLLMGCVADIVLTQTPSSLSASLGDRVTISCRASQDISNYLNW
YQLKPDGTVKLLIYYTSRLHSGVPSRFSGSGSGTDYSLTISNLEQEDIATYFCQQGNTLPPT
FGGGTKLEIKRADAAPTVS
21) Anti gp68 clone 68.06
Heavy chain variable region polypeptide sequence, 158 amino acids
FR1-CDR1-FR2-CDR2-FR3-CDR3-FR4
SEQ 68.06 HC8_.ab1:
(SEQ ID NO: 54)
MGILPSPGMPALLSLVSLLSVLLMGCVAQVKLEQSGAELMKPGASVKISCTATGYTFSGYWI
EWVKQRPGHGLEWIGEILPGSGGTNYNEKFKGKATFTADTSSNTAYMQLSSLTSEDSAVY
YCARGIHYFGYYYFDYWGQGTTLTVSSAKTTPPSVY
Light chain variable region polypeptide sequence, 144 amino acids
FR1-CDR1-FR2-CDR2-FR3-CDR3-FR4
SEQ 68.06 LC2_.ab1:
(SEQ ID NO: 55)
MGILPSPGMPALLSLVSLLSVLLMGCVADIVLTQSPSSLSASLGDRVTISCRASQDISNYLNW
YQLKPDGTVKLLIYYTSRLHSGVPSRFSGSGSGTDYSLTINNLEQEDIATYFCQQGNTLPPT
FGGGTKLEIKRADAAPTVS
22) Anti gp68 clone 68.07
Heavy chain variable region polypeptide sequence, 159 amino acids
FR1-CDR1-FR2-CDR2-FR3-CDR3-FR4
SEQ 68.07 HC1_.ab1:
(SEQ ID NO: 56)
MGILPSPGMPALLSLVSLLSVLLMGCVAEVKLVQSGGGLVQPGGSLRLSCATSGFTFSDFY
MEWVRQPPGKRLEWIAASRNKANDYATEYSASVKGRFIVSRDTSQSILYLQMNALRAEDT
AIYYCARDAGDYWGYFNVWGAGTTVTVSSAKTTPPSVY
Light chain variable region polypeptide sequence, 150 amino acids
FR1-CDR1-FR2-CDR2-FR3-CDR3-FR4
SEQ 68.07 LC1_.ab1:
(SEQ ID NO: 57)
MGILPSPGMPALLSLVSLLSVLLMGCVADIVMTQTPSSLGVSVGEKVTMSCKSSQSLLYSS
NQKNYLAWYQQKPGQSPKLLIYWASTRESGVPDRFTGSGSGTDFTLTISSVKAEDLAVYY
CQQYYSYPLTFGAGTKLELKRADAAPTVS
23) Anti gp68 clone 68.08
Heavy chain variable region polypeptide sequence, 159 amino acids
FR1-CDR1-FR2-CDR2-FR3-CDR3-FR4
SEQ 68.08 HC8_.ab1:
(SEQ ID NO: 58)
MGILPSPGMPALLSLVSLLSVLLMGCVAQVQLVESGGGLVQPGGSLRLSCATSGFTFSDFY
MEWVRQPPGKRLEWIAASRNKANDYATEYSASVKGRFIVSRDTSQSILYLQMNALRAEDT
AIYYCARDAGDYWGYFNVWGAGTTVTVSSAKTTPPSVY
Light chain variable region polypeptide sequence, 150 amino acids
FR1-CDR1-FR2-CDR2-FR3-CDR3-FR4
SEQ 68.08 LC4_.ab1:
(SEQ ID NO: 59)
MGILPSPGMPALLSLVSLLSVLLMGCVADIVITQSPSSLGVSVGEKVTMSCKSSQSLLYSSN
QKNYLAWYQQKPGQSPKLLIYWASTRESGVPDRFTGSGSGTDFTLTISSVKAEDLAVYYC
QQYYSYPLTFGAGTKLELKRADAAPTVS.

In the above represented sequences, the CDRs are underlined and the amino acids forming the CDRs are printed in bold. From the sequences it is evident for a person skilled in the art how the CDRs are oriented. Moreover, it goes without saying that it is possible to combine the CDRs as shown in the above sequences with each other. The sequences and the CDRs must match together. The above provided sequences represent preferred embodiments of the invention.

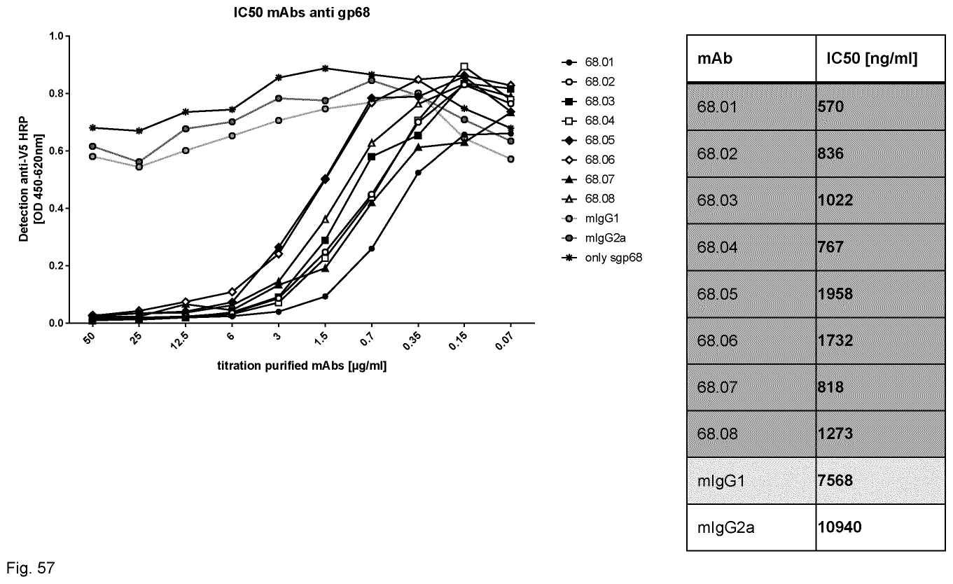

FIG. 57 shows the efficiency of the preferred antibodies directed against glycoprotein gp68. As controls the following antibodies were used: mIgG1, mIgG2a and only sgp68. It can be seen that no inhibition takes place. By using the preferred antibodies against gp68, namely 68.01; 68.02; 68.03; 68.04; 68.05; 68.06; 68.07 and 68.08 a substantial inhibition could be obtained. The IC50 values for the different monoclonal antibodies have been measured whereby the best results were obtained with mAb 68.01.

### Characterization of Anti-vFcγR moAbs in Flow Cytometry (FACS)

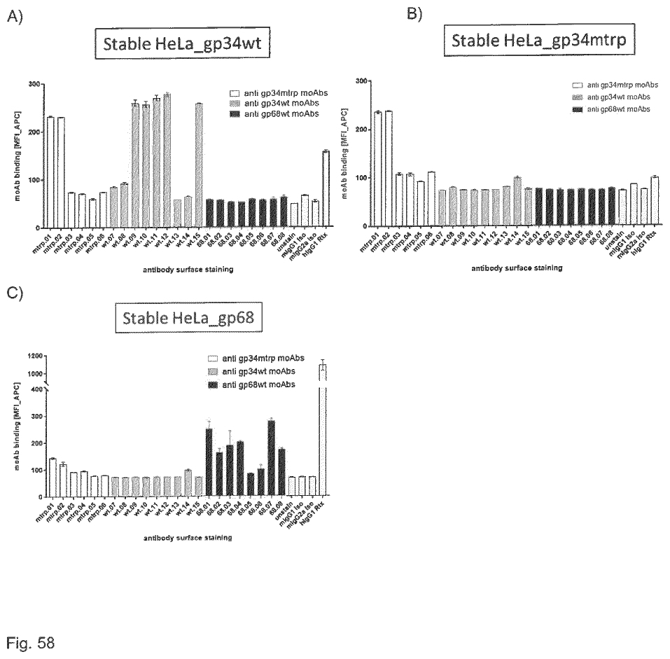

Beside the application of the moAbs in ELISA and western blot (WB) it was tested whether the moAbs are also suitable for application in flow cytometry using transfected cells. Specifically, the potential of moAbs to recognize plasma membrane exposed gp34wt, gp34mtrp or gp68 on stably transduced Hela cells. Indicated moAbs (1 μg/ml) or control antibodies were incubated with Hela cells either expressing the gp34wt protein (derived from HCMV strain AD169), the point mutant gp34mtrp (deficient for IgG Fc-binding, containing one amino acid exchange at position 65 from Trp to Phe) or gp68 (derived from HCMV strain AD169). After an incubation time of 1 hour on ice, cells were washed and stained with a secondary goat anti mouse IgG-APC labeled antibody. Staining was performed one hour on ice, before cells were washed and analyzed (FACS Fortessa). A representative data set out of 3 experiments is shown in FIG. 58.

FIG. 58 is a characterization of anti-vFcγR moAbs in flow cytometry using stably transduced Hela cells. Monoclonal antibodies were tested for their ability to recognize surface exposed gp34wt (A), gp34mtrp (B) or gp68 (C) expressed by stably transduced HeLa cells as indicated.

Monoclonal antibodies as indicated were incubated with respective stable cells. Binding of respective antibodies was determined using secondary goat anti-mouse IgG-APC (or goat anti human IgG-APC for Fc binding control) measured by FACS Fortessa. Human IgG1 (Rituximab, RTX) followed by goat anti-human IgG-APC is used as a positive control for IgG-binding in case of gp34wt and gp68. Mouse IgG isotype control antibodies served as a negative control. gp34mtrp is impaired for IgG-binding.

The binding studies using flow cytometry identified the following anti-gp34 moAbs to have the potential to recognize native folded gp34wt antigen on the cell surface: mtrp.01/02,wt.09/10/11/12 and wt.15 (see FIG. 58A). While all moAbs were proved to be antigen-antigen specific before in ELISA and Western blot (WB), these moAbs identified here are additionally suitable for application in flow cytometry (FIG. 58A).

As shown before in ELISA and WB, none of the anti-gp34 moAbs immunized by the gp34wt protein are capable to recognize the gp34mtrp protein. Accordingly, moAbs gp34wt.07-gp34wt.15 did not bind to Hela cells expressing the gp34mtrp protein by FACS (FIG. 58B). With regard to the anti-gp34mtrp moAbs only the pair gp34mtrp.01/02 was able to bind in flow cytometry (FIG. 58A, B) which is in clear contrast to the earlier ELISA data, where all anti-gp34mtrp moAbs did recognize the gp34mtrp protein with a similar efficacy. These data demonstrate that the anti-gp34mtrp moAbs have different conformational requirements for binding.

Next, we characterized the anti-gp68 moAbs in flow cytometry with the following order of signal strength: gp68.07>gp68.01>gp68.02/gp68.03/gp68.04/gp68.08 (FIG. 58C). This result confirms the results obtained before in ELISA binding studies and titration experiments. In conclusion, anti-gp68.01/.02/.03/.04/.07/.08 are suitable for flow cytometry based assays.

The anti-gp68 monoclonal antibodies gp68.05/gp68.06 were unable to detect gp68 on the plasma membrane of stably transduced cells. Both moAbs were also weaker in gp68 detection by ELISA, but in contrast to the FACS data still able to recognize gp68 antigen in the ELISA detection system (see earlier results). In conclusion, for flow cytometry analysis anti-gp68.05/.06 are not applicable.

### Epitope Mapping for Anti-gp68 moAbs

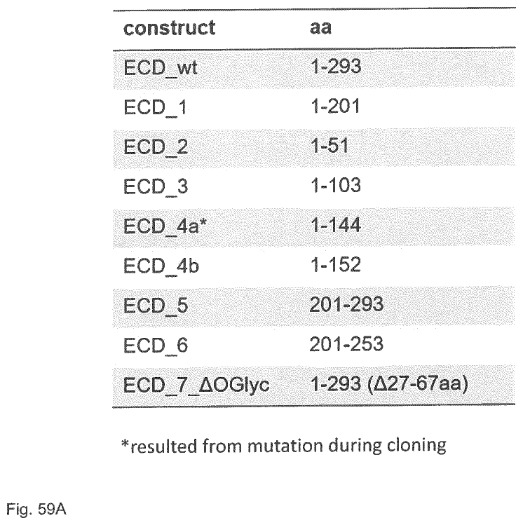

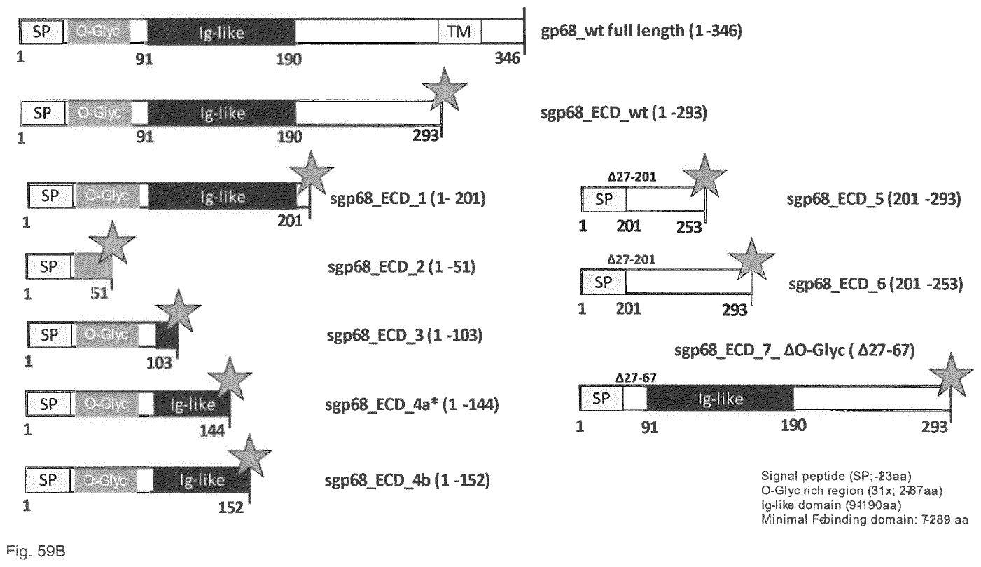

To narrow down the antigenic regions on gp68 recognized by the anti-gp68 moAbs we generated several truncation mutants of the gp68wt extracellular domain (gp68_ECD_wt). Plasmids were used for transfection of HEK293T cells using a polyethylenimine (PEI)-based transfection protocol. gp68 proteins comprising the ECD but lacking the transmembrane segment (TM) and cytoplasmic tail are secreted into the cell supernatant. In a first set of experiments, supernatants were used for direct coating of ELISA plates. In FIG. 59A ECD-constructs are depicted. ECD_wt and ECD_variants as indicated were successfully cloned and the correct sequence was confirmed by DNA sequencing. All truncation variants of the gp68_ECD_wt contain a C-terminal 6xHis-epitope Tag to allow for expression control.

FIGS. 59A and B are an overview cloning strategy gp68_ECD_wt and truncation variants thereof. Several variants of gp68_ECDs were planned to narrow down potential epitopes recognized by anti-gp68 moAbs (A).

Schematic representation of gp68_ECD constructs is highlighted and C-terminal 6× His-Tag is indicated (grey star, B). The signal peptide (SP, light grey box), an O-glycosylation-rich region located in the N-terminal part of gp68 is indicated (O-Glyc, dark grey box), as well as a predicted Ig-like domain (Ig-like, black box).

All gp68_ECD constructs were successfully cloned and correct DNA sequence was confirmed by sequencing are highlighted and a C-terminal 6× His-Tag is indicated (grey star, in FIG. 59B).

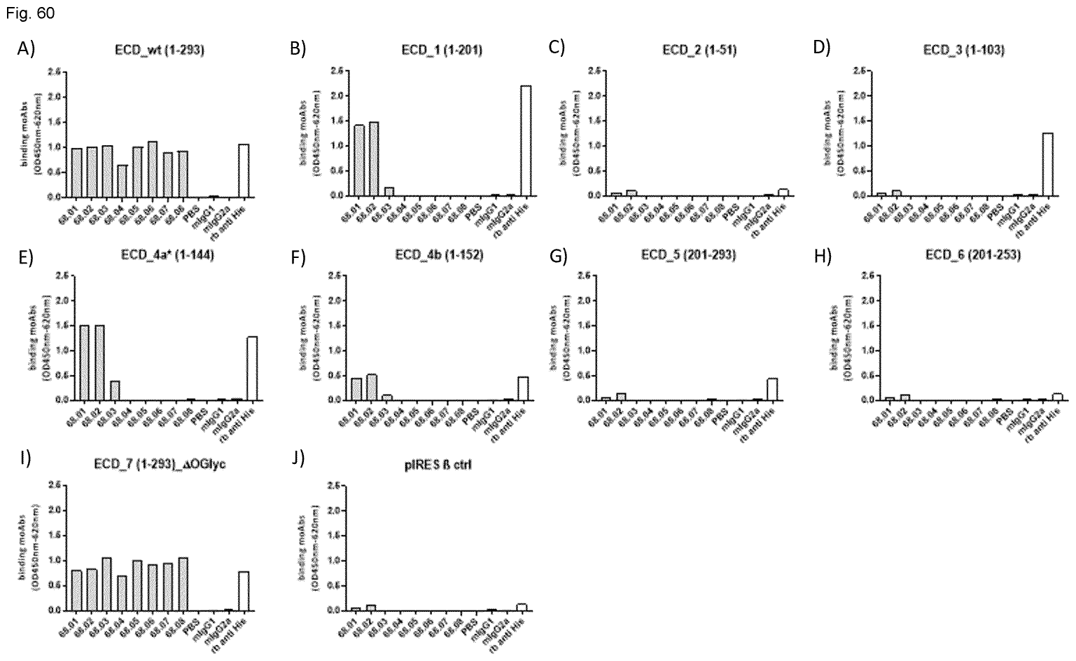

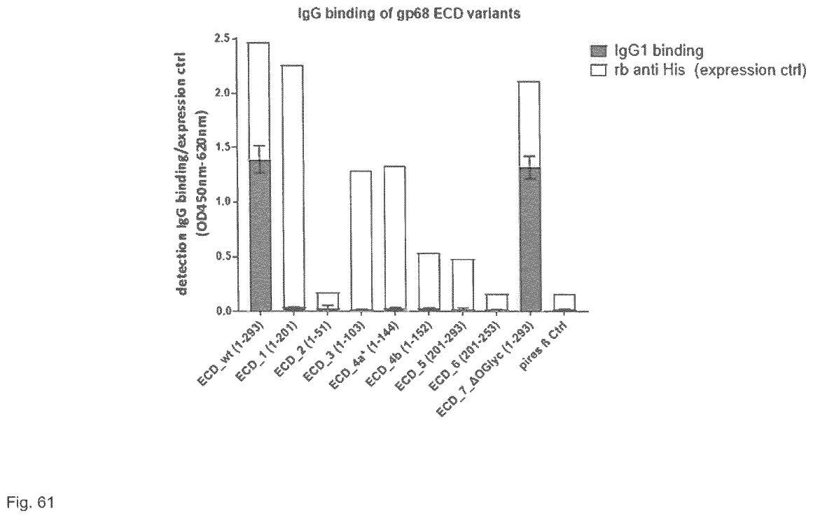

200 μl/well supernatant harvested on day 4 after transfection of HEK293T was used for coating. After blocking, plates were incubated with 1 μg/ml anti-gp68 moAbs, as well as mouse IgG1 and mouse IgG2a as isotype controls and mouse anti-His-Tag for control of protein expression after transfection. Binding of moAbs was determined using goat anti-mouse IgG conjugated to POD (horse radish peroxidase, HRP; GAM-POD 1:3000). Plates were measured at wavelength 450-620 nm in Tecan reader. Binding pattern of all anti-gp68 moAbs to gp68_ECD_wt (same antigen as used for immunization) is similar to that measured by ELISA assays earlier (FIG. 60A). There is no unspecific binding seen when incubated on supernatants of HEK293T cells transfected with empty plasmid (pIRES_ß_EGFP Ctrl, FIG. 60B). Supernatants containing potential gp68_ECD_2 (1-51aa) and gp68_ECD_6 (201-253aa) revealed that the proteins were not released into the supernatant or not stable after synthesis albeit the sequence was confirmed to be correct (from Start codon to C-terminal 6×His Tag). Gp68_ECD_1, gp68_ECD_3, gp68_ECD_4* (1-144aa, due to mutation event that occurred during cloning a stop codon was inserted shortening the originally planned gp68-ECD4 construct), gp68_ECD4b (1-52aa) and gp68_ECD_7 (1-293, lacking highly O-glycosylation region on the N-terminus of gp68, 27-67aa) however, were expressed. Binding to epitope regions covered by gp68-ECD_1, gp68_ECD4a* and gp68_ECD4b could be confirmed for anti-gp68 moAbs 68.01 and 68.02 (FIG. 60C and FIG. 61C). All anti-gp68 moAbs recognized gp68_ECD_wt as well as gp68_ECD_7 (gp68 AO-glyc, lacking the highly O-glycosylation region at the N-terminus of gp68, 27-67aa) with the same strength, indicating also that none of the anti-gp68 antibodies recognized this particular region.

FIG. 60 shows binding to direct-coated gp68_ECD_wt and truncation variants thereof by anti-gp68 moAbs. Several truncation variants of gp68 were expressed after HEK293T transfection and secreted into cell culture supernatants. Supernatants were used for direct coating onto ELISA plates and incubated with indicated anti-gp68 moAbs. Binding to gp68_ECD_1 (B), gp68_ECD_2 (C), gp68_ECD_3 (D), gp68_ECD_4a* (E), gp68_ECD_4b (F), gp68_ECD_5 (G), gp68_ECD_6 (H) and gp68_ECD_7 (I) was detected using goat anti mouse IgG POD antibody. For expression control of gp68_ECDs rabbit anti His primary antibody was used followed by goat anti rabbit IgG POD antibody. After addition of substrate, plates were measured using Tecan reader. Binding is compared to supernatants containing gp68_ECD_wt (A) as positive control. No unspecific binding was observed to supernatants from HEK293T cells transfected with empty plasmid control (J). All anti-gp68 moAbs showed similar binding to gp68_ECD_wt and gp68_ECD_7 (gp68 ΔO-glyc, lacking the highly O-glycosylated region at the N-terminus of gp68, 27-67aa; FIG. 60 I). Only moAbs 68.01/.02 showed in addition binding to ECD_1 (B), ECD_4a* (E) and ECD_4b (F) comprising amino acids 1-152 of gp68.

To test IgG-Fc binding potential of generated gp68 ECDs, SNs generated after293T transfection as mentioned above were directly coated into ELISA plates and either incubated with human IgG1 (Sigma Aldrich, 1 μg/ml) or without. IgG binding by gp68_ECD_wt and gp68 ECDs variants as indicated was measured and analyzed. For expression control, SNs of respective supernatants containing gp68_ECD_wt or gp68_ECD_variants after transfection were detected using rabbit anti-His antibody followed by goat-anti rabbit IgG POD and measured using Tecan reader.

FIG. 61 shows IgG-binding to direct-coated gp68_ECD_wt and truncation variants thereof. Several truncation variants of gp68 were expressed after HEK293T transfection and secreted into cell culture supernatants. Supernatants were used for direct coating onto ELISA plates and incubated human IgG (1 μg/ml) or rabbit-anti His antibody for expression control. After addition of substrate, plates were measured using Tecan reader. IgG-binding is compared to supernatants containing gp68_ECD_wt as positive control. Supernatants from HEK293T cells transfected with empty plasmid control served as negative control (no IgG binding). Only gp68_ECD_wt and gp68_ECD_7 (gp68 ΔO-glyc, lacking highly O-glycosylation region on the N-terminus of gp68, 27-67aa) show IgG-binding whereas all other gp68_ECDs lost their IgG-binding ability. Data for expression of His-tagged proteins in supernatants (white bars) combined to IgG binding potential (black bars) is shown. Presumably, gp68-ECD_1 to gp68_ECD_6 are impaired for IgG binding. Compared to gp68_ECD_wt, only gp68_ECD_7 (ΔO-glyc) kept IgG-binding ability. All anti-gp68 moAbs recognize in addition to gp68wt, also gp68 ΔO-glyc, lacking the highly O-glycosylated region at the N-terminus of gp68. Due to our results, this region on gp68 seems to be negligible for IgG-binding by gp68. Also, previous experiments by our group determined the minimal IgG-Fc-binding domain of gp68, comprising amino acids 68-289 aa (lacking the putative highly O-glycosylated-rich protein region (31× putative O-glycosylation sites) at the N-terminus of gp68, but apart from that covering the whole remaining ECD of gp68, including the predicted Ig-like domain (91-190 aa).

In conclusion, the data indicate that anti-gp68 moAb 68.01/.02 recognize an epitope different from the other anti-gp68 moAbs and that the epitope recognized is located within amino acids 1-152 of the ectodomain (ECD_4). It is tempting to speculate that anti-gp68 moAbs 68.03-68.08 recognize an epitope located within 202-293 amino acid, due to the fact that none of them bound to gp68_ECD_1 (1-201 aa) as 68.01/68.02 did. It is also possible that anti-gp68moAbs 68.03-68.08 recognize a structural epitope not formed by each individual gp68_ECD variant.

In summary, epitopes on the gp68 protein can be further narrowed down regarding our ELISA data for anti-gp68 moAbs as the following:
(i) epitopes recognized by anti-gp68.01/.02 comprises amino acids: 1-201 (ECD_1; EC_4a* and ECD_4b)
(ii) all anti-gp68 moAbs also recognize a gp68 variant gp68 (ΔO-glyc) lacking a O-glycosylation-rich region at the N-terminus of gp68 (ECD_7, lacking the highly O-glycosylated region at the N-terminus of gp68, 27-67aa).

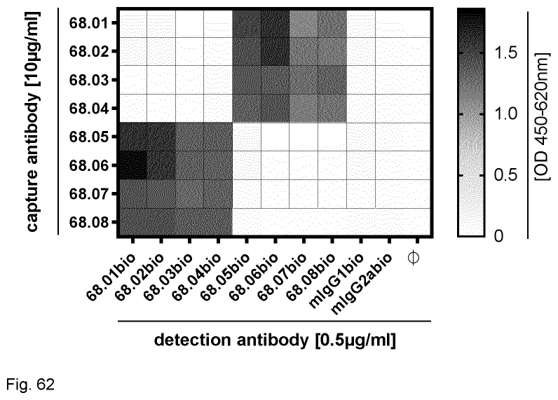

To further narrow down potential epitopes, to compare independent moAb recognizing gp68 and thereby distinguishing the anti-gp68 moAbs further, we decided to perform competition ELISAs based on a sandwich set up using unconjugated anti-gp68 moAbs for coating in a concentration of 10 μg/ml followed by the addition of the respective anti-gp68 moAbs in their biotinylated form (0.5 μg/ml). Only biotinylated anti-gp68 moAbs that do not compete with the capture moAbs should be able to detect recombinant sgp68wt protein added to this sandwich ELISA setup (see FIG. 62) in an unimpaired manner.

In brief, capture moAbs were coated at 10 μg/ml overnight as indicated. After blocking, 1 μg/ml recombinant sgp68wt protein was added and allowed for binding overnight at 4° C. Plates were washed and biotinylated anti-gp68 detection moAbs-were added (0.5 μg/ml for 30 mins only). After intense washing, bound moAbs were detected using Strept-POD. Plates were analyzed using Tecan reader (OD 450-620 nm).

Based on the competition ELISA we concluded that when anti-gp68 moAbs 68.01-68.04 are used as capture antibody, only anti-gp68 moAbs 68.05-68.08 are potent to to bind. The same is true vice versa, when anti-gp68 moAbs 68.05-68.08 are already bound to gp68 protein, binding by anti-gp68 moAbs 68.01-68.04 is not abolished indicating that 68.01-68.04 and 68.05-68.08 recognized at least overlapping epitopes, respectively.

FIG. 62 is a competition sandwich-ELISA using anti-gp68 moAbs as capture and anti-gp68-biotinylated moAbs for detection. As indicated, unconjugated anti-gp68 moAbs were used for coating (10 μg/ml) overnight, followed by incubation with purified sgp68wt protein as antigen. Biotinylated anti-gp68 moAbs were added at 0.5 μg/ml for 30 mins. After washing, Strept-POD was used to determine binding of the detection antibodies towards gp68wt protein. Biotinylated mouse IgG1 and IgG2a were used as negative control (n=3 replicates).

In conclusion, also based on our data from the ECD-epitope mapping, we suggest the following groups of anti-gp68 moAbs:
Group I: 68.01/68.02 (the only anti-gp68 moAbs detecting also denatured gp68 antigen in western blot analysis)
Group II: 68.03/68.04 (recognize both gp68_ECD4 comprising amino acids 1-152aa)
Group III: 68.05/68.06/68.07/68.08

### Epitope Mapping for Anti-gp34wt and Anti-gp34mtrp moAbs

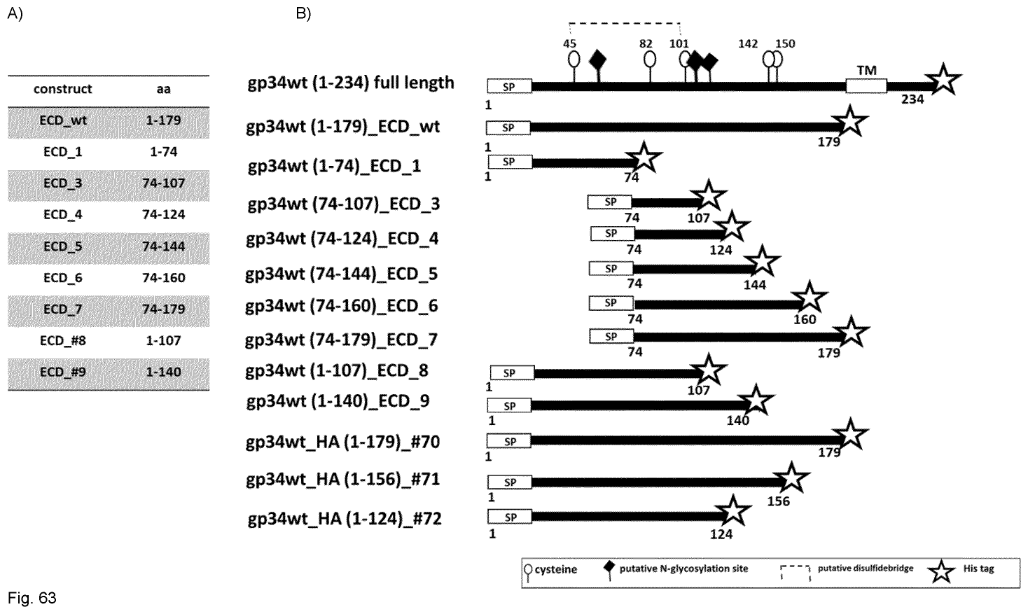

To narrow down the epitopes on gp34wt and gp34mtrp protein recognized by the anti-gp34 moAbs several truncation mutants of the gp34wt extracellular domain (gp34_ECD_wt) were generated. Plasmids were used for transfection of either HEK293T or Hela cells, as indicated, using the PEI transfection protocol. gp34 mutants consisting of the ECD and lacking the transmembrane (TM) domain and cytoplasmic tail are expected to be secreted into the supernatant of cells. In a first set of experiments, supernatants were either used for direct coating or for coating onto Nickel-plates as indicated. An overview of planned ECD-constructs is listed in FIG. 63. All truncation variants of the gp34_ECD_wt contain a C-terminal 6×His-Tag to allow for expression control.

FIG. 63 is an overview of the cloning strategy of gp34_ECD_wt and truncation variants thereof. Several variants of gp34wt protein (comprising amino acids 1-234), named gp34_ECDs, were generated to narrow down potential antigenic regions recognized by anti-gp34 moAbs (A). Schematic representation of gp34_ECD constructs are highlighted and C-terminal 6×His-Tag is indicated (open star, B). Cysteines are indicated by open circle, putative N-glycosylation sites are shown in black diamonds and a putative disulfide-bride is indicated in dotted line.

Expression of cloned gp34_ECD_wt and truncated variants thereof was tested in western blot and dot blot experiments. gp34_ECD_1 and gp34_ECD_3 seem to be not sufficiently stable for detection albeit DNA sequence was confirmed to be correct. This could be due to instable gp34 truncation molecules.

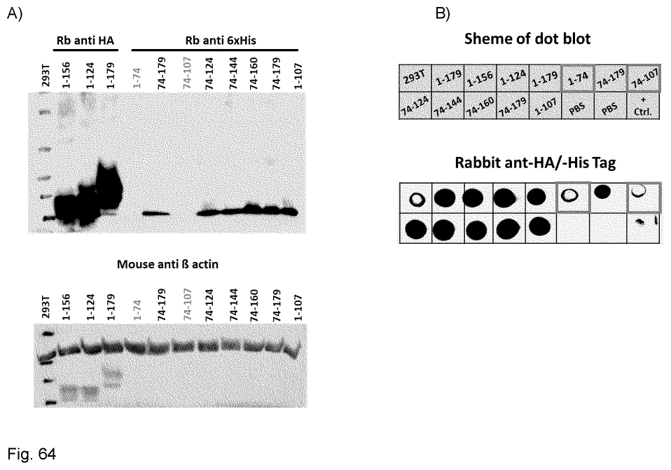

FIG. 64 shows the detection of protein expression of gp34_ECD_wt and truncation variants thereof. Several variants of gp34_ECDs were generated to narrow down potential epitopes recognized by anti-gp34 moAbs to distinguish moAbs among each other. Hela cells were transfected with indicated plasmids encoding for various gp34_ECDs. Cell lysates were analyzed in western blot using anti-Tag antibodies (either HA-Tag or 6×His-Tag) or mouse anti-ß actin as loading control. In addition, supernatants of transfected cells expressing various gp34_ECDs were investigated in dot blot assays using rabbit-anti HA or rabbit anti His-antibody, followed by goat-anti rabbit IgG POD for detection. Except for gp34 ECD_1 (1-74aa) and gp34_ECD_3 (indicated by grey boxes) expression of proteins could be demonstrated.

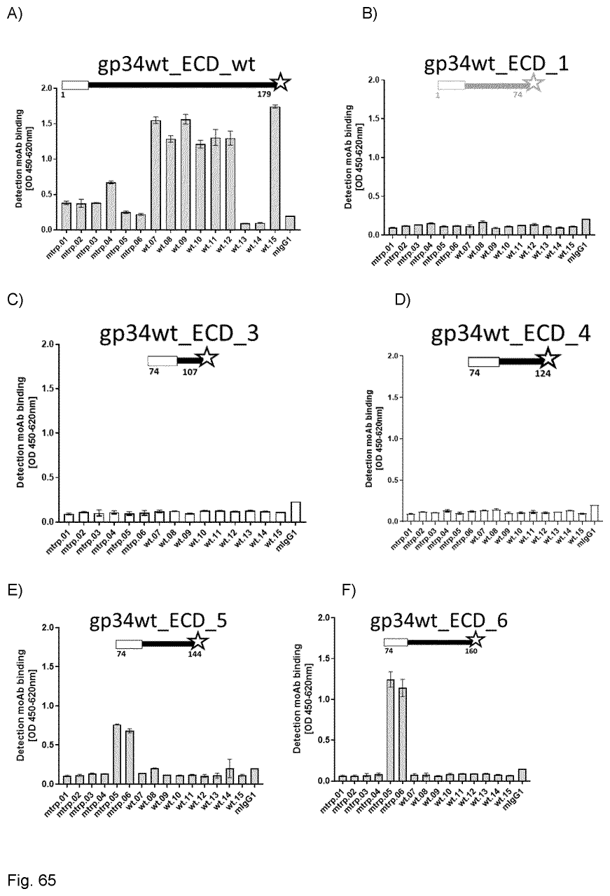

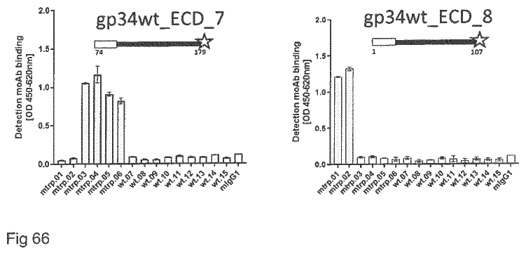

200 μl/well of supernatant after transfection of Hela cells (harvested at d4 after transfection) were used for coating. After blocking, plates were incubated with 1 μg/ml anti-gp34mtrp or anti-gp34wt moAbs, as well as mouse IgG1 as isotype control. Binding of moAbs was determined using goat anti-mouse IgG conjugated to POD (horse radish peroxidase, HRP; GAM-POD 1:2000). Plates were measured at wavelength 450-620 nm in Tecan reader. Binding pattern of all anti-gp34 moAbs to gp34_ECD_wt (same antigen as used for immunization) is similar to that seen in ELISA assays earlier. There is no unspecific binding seen when incubated with supernatants of Hela cells transfected with empty plasmid (pires_ß_EGFP). Supernatants containing potential gp34_ECD_1 and gp34_ECD_3 (see FIGS. 65 and 66) seem to be not expressed (as based on WB and dot blot results, (see FIG. 64) or not released to the supernatant albeit the sequence was correct (from start codon to C-terminal 6×His Tag).

FIG. 65 is a binding to direct-coated gp34_ECD_wt and truncation variants thereof by anti-gp34 moAbs. Several truncation variants of gp34 were expressed after transfection of HeLa cells and secreted into cell culture supernatant. Supernatants were used for direct coating onto ELISA plates and incubated with indicated anti-gp34 moAbs. Binding to gp34_ECD_1 (B), gp34_ECD_3 (C), gp34_ECD_4 (D), gp34_ECD_5 (E), gp34_ECD_6 (F), and FIG. 66, gp34_ECD_7 (G) and gp34_ECD_8 (H) was detected using goat anti mouse IgG POD antibody. After addition of substrate, plates were measured using Tecan reader. Binding is compared to supernatants containing gp34_ECD_wt (A) as positive control. Except to gp34_ECD_wt only anti-gp34mtrp moAbs showed binding to selective, truncated gp34_ECDs as shown allowing to further narrow down epitopes recognized by anti-gp34mtrp moAbs. None of the anti-gp34wt moAbs was able to recognize truncated forms of gp34_ECD.

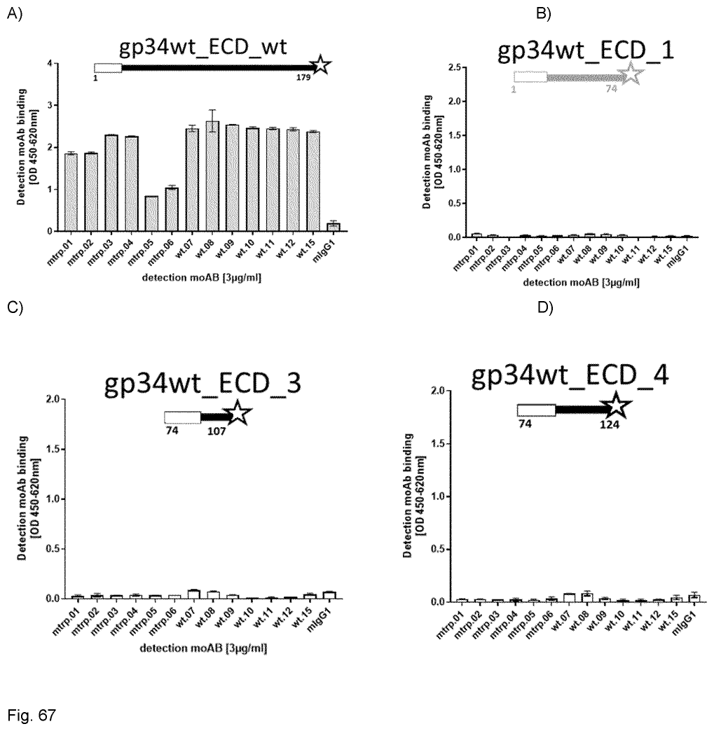

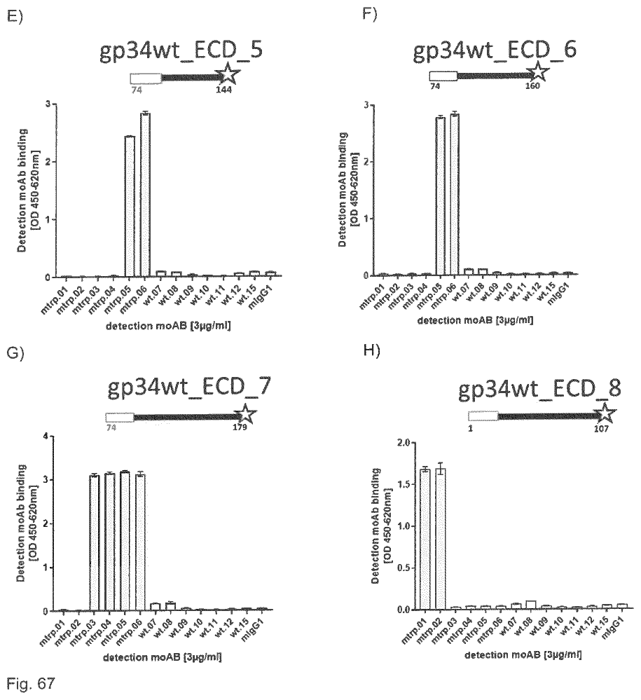

FIG. 66 and FIG. 67 show binding of anti-gp34 moAbs to nickel-coated gp34_ECD_wt and truncation variants thereof. Several truncation variants of gp34 were expressed after HeLa transfection and secreted into cell culture supernatant. Supernatants were used for binding to nickel-plates and incubated with indicated anti-gp34 moAbs. Binding to gp34_ECD_1 (B), gp34_ECD_3 (C), gp34_ECD_4 (D), gp34_ECD_5 (E), gp34_ECD_6 (F), gp34_ECD_7 (G) and gp34_ECD_8 (H) was detected using goat anti mouse IgG POD antibody. After addition of substrate, plates were measured using Tecan reader. Binding is compared to supernatants containing gp34_ECD_wt as positive control. Except to gp34_ECD_wt only anti-gp34mtrp moAbs showed binding to selective, truncated gp34_ECDs as shown allowing to further narrow down epitopes recognized by anti-gp34mtrp moAbs. None of the anti-gp34wt moAbs was able to recognize truncated forms of gp34_ECD as seen before (see FIG. 65).

In summary, epitopes on gp34wt protein can be further narrowed down regarding our ELISA data for anti-gp34mtrp moAbs as the following:
(i) epitopes recognized by anti-gp34mtrp.01/.02 comprises amino acids: 1-107
(ii) epitopes recognized by anti-gp34mtrp.03/.04 comprises amino acids: 74-179
(iii) epitopes recognized by anti-gp34mtrp.01/.02 comprises amino acids: 74-144
(iv) epitopes recognized by anti-gp34wt.07-gp34wt.15 is only present in sgp34wt comprising amino acids 1-179, indicating potentially structural epitopes.

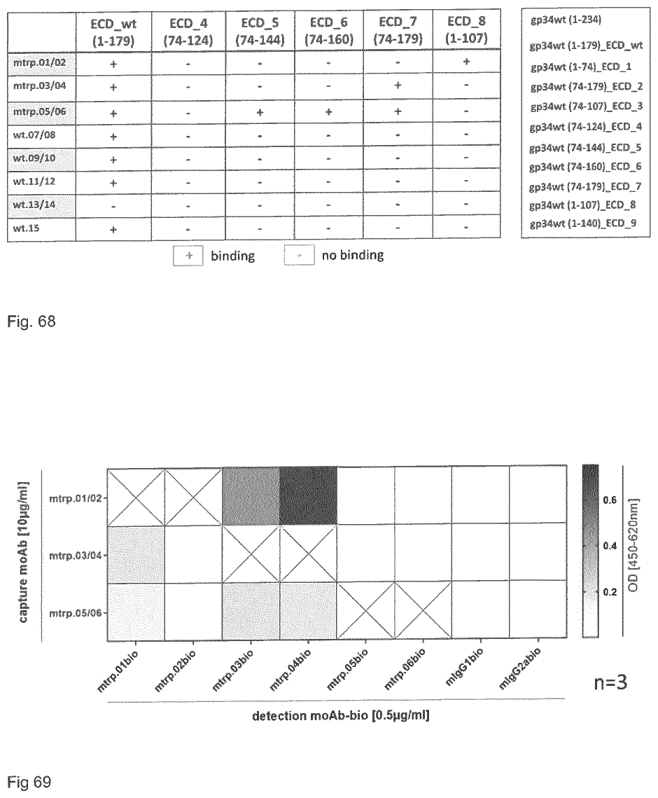

FIG. 68 is a summary of anti-gp34 moAbs binding to various gp34wt_ECDs_. Several truncation variants of gp34 were expressed after transfection of Hela cells and secreted into cell culture supernatant. Supernatants were analyzed regarding anti-gp34 moAb binding in ELISA and summarized results are illustrated. Binding of gp34_ECD_wt served as positive control (same protein was used for immunization) and comprises amino acids 1-179. Binding by respective moAbs is highlighted (+). No binding by the moAb is also listed in the table (−).

To narrow down potential epitopes and to distinguish binding requirements between the anti-gp34 moAbs further, a competition ELISAs approach was pursued which was based on a sandwich set up using unconjugated anti-gp34 moAbs for coating in a concentration of 10μg/ml followed by the addition of the respective anti-gp34 moAbs in their biotinylated form (0.5μg/ml). Only biotinylated anti-gp34 moAbs that do not compete with the capture moAbs should be able to detect recombinant sgp34wt protein added to this sandwich ELISA setup.

Capture moAbs were coated at 10 μg/ml overnight as indicated. After blocking, 1 μg/ml recombinant sgp34wt protein was added and allowed for binding overnight at 4° C. Plates were washed and respective anti-gp34 moAbs in their biotinylated form were added (0.5 μg/ml for 30 mins only). After intense washing, bound moAbs-biotinylated were detected using Strept-POD. Plates were analyzed using Tecan reader (OD 450-620 nm).

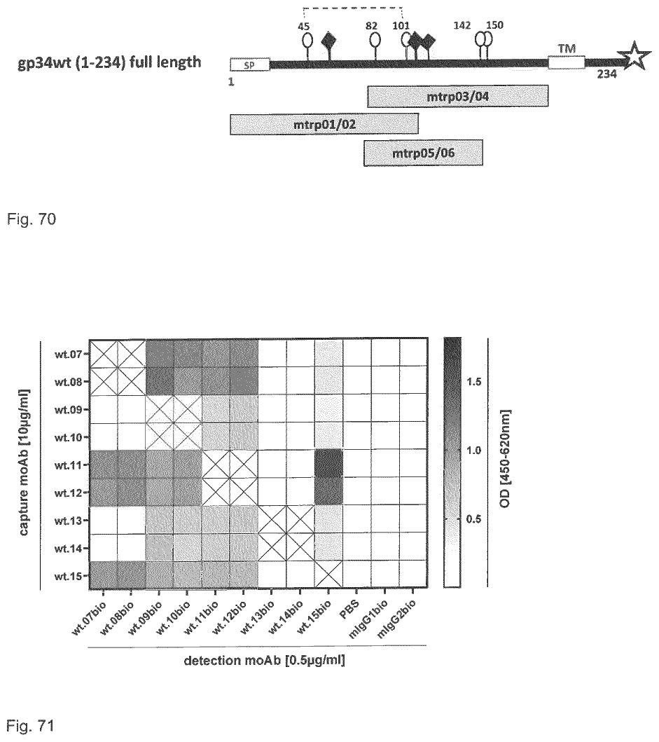

Results for competition assays are shown for anti-gp34mtrp moAbs (FIGS. 68 and 69) and for anti-gp34wt moAbs (FIG. 70).

FIG. 69 is a competition sandwich-ELISA using anti-gp34mtrp moAbs as capture and anti-gp34mtrp-biotinylated moAbs for detection. As indicated, unconjugated anti-gp34mtrp moAbs were used for overnight coating (10 μg/ml), followed by incubation with purified sgp34wt protein. Anti-gp34mtrp moAbs in their biotinylated form were added at 0.5 μg/ml for 30 mins. After washing, Strept-POD was used to determine binding of the detection antibodies towards gp34wt protein. Biotinylated mouse IgG1 and IgG2a were used as negative control (n=3replicates). Combinations in which the same pairs of antibodies were used in its unlabeled version (capture) and in its biotinylated form (detection) are indicated (X).

Based on the data from competition assays and from bindings assays using various gp34wt-ECDs mutants we suggest the following distinction of anti-gp34mtrp moAbs. A schematic representation of the epitope mapping is summarized in FIG. 70).
(i) mtrp.01/.02 bind to sgp34wt protein when mtrp.03/.04/.05/.06 are already pre-bound
(ii) mtrp.03/.04 bind strong to sgp34wt protein when mtrp.01/.02 are already pre-bound
(iii) mtrp.03/.04 bind to sgp34wt protein when mtrp.05/.06 are already pre-bound but binding is affected, indicating interference with mtrp.05/.06 binding
(iv) mtrp.05/.06bio binding is weaker, as seen before in ELISA binding experiments before, but different from mtrp.01/02 and mtrp.03/.04.

FIG. 70 is an antigenic region of gp34wt as mapped for anti-gp34mtrp moAbs. Potential epitopes for anti-gp34mtrp moAbs on the gp34wt full length protein (amino acids 1-234) are indicated (grey boxes, followed by names of the moAbs). Assumptions are based on ELISA binding studies using various gp34wt-ECDs and on competition ELISAs using unconjugated moAbs for capture and biotinylated moAbs for detection of gp34wt. The predicted signal peptide (SP) and transmembrane region (TM) is illustrated. C-terminal 6×His-Tag is indicated (open star). Cysteines are indicated by open circle, putative N-glycosylation sites are shown in black diamonds and a putative disulfide-bride is indicated by dotted line.

Based on the data from competition assays and from bindings assays using various gp34wt-ECDs mutants it is suggested the following distinction of anti-gp34mtrp moAbs. A schematic representation of the epitope mapping is summarized in FIG. 70).
(i) mtrp.01/.02 bind to sgp34wt protein when mtrp.03/.04/.05/.06 are already pre-bound
(ii) mtrp.03/.04 bind strong to sgp34wt protein when mtrp.01/.02 are already pre-bound
(iii) mtrp.03/.04 bind to sgp34wt protein when mtrp.05/.06 are already pre-bound but binding is affected, indicating interference with mtrp.05/.06 binding
(iv) mtrp.05/.06bio binding is weaker, as seen before in ELISA binding experiments before, but different from mtrp.01/02 and mtrp.03/.04.

FIG. 70 shows the antigenic regions of gp34wt as mapped for anti-gp34mtrp moAbs. Potential epitopes for anti-gp34mtrp moAbs on the gp34wt full length protein (amino acids 1-234) are indicated (grey boxes, followed by names of the moAbs). Assumptions are based on ELISA binding studies using various gp34wt-ECDs and on competition ELISAs using unconjugated moAbs for capture and biotinylated moAbs for detection of gp34wt. The predicted signal peptide (SP) and transmembrane region (TM) is illustrated. C-terminal 6×His-Tag is indicated (open star). Cysteines are indicated by open circle, putative N-glycosylation sites are shown in black diamonds and a putative disulfide-bride is indicated by dotted line.

FIG. 71 is a competition sandwich-ELISA using anti-gp34wt moAbs as capture and (biotinylated) anti-gp34wt moAbs for detection. As indicated, unconjugated anti-gp34mtrp moAbs were used for overnight coating (10 μg/ml), followed by incubation with purified sgp34wt protein. Anti-gp34wt moAbs in their biotinylated form were added at 0.5 μg/ml for 30 mins. After washing, Strept-POD was used to determine binding of the detection antibodies towards gp34wt protein. Biotinylated mouse IgG1 and IgG2a were used as negative control (n=3 replicates). Combinations were antibodies from the same moAb were used in its unlabeled version (capture) and in its biotinylated form (detection) are indicated (X).

Altogether, based on the competition ELISA presented herein it can be concluded that complex interaction pattern exist regarding the binding of independent gp34wt moAbs, which could be due to overlapping epitopes or conformational changes induced upon moAb binding when compared to anti-gp34mtrp moAbs.

From above experiments the following can be concluded:
(i) wt.07/.08 still bind in parallel to gp34wt protein when wt. 11/.12 and wt.15 already pre-bound
(ii) wt.07/.08 binding is abrogated when wt.09/.10/.13/.14 are already pre-bound
(iii) wt.09/.10/11/12 still show strong binding independent of competitor antibody used for capture
(iv) wt. 13/14 show no binding, as seen in binding ELISAs before
(v) wt.15 strongly binds in parallel to gp34wt protein when wt.11/.12 are already pre-bound
(vi) wt.15 binding is reduced when wt.07/08/.13/.14 are already pre-bound to sgp34wt protein
(vii) wt.15 binding is most affected when wt.09/10 are already pre-bound to sgp34wt protein

Based on the data from the ECD-epitope mapping and the competition ELISA experiments, the following grouping of anti-gp34 moAbs considering the binding studies and the competition assay data is suggested:
Group I: mtrp.01/02 (recognize an epitope on gp34wt protein comprising amino acids 1-107aa)
Group II: mtrp.03/04 (recognize an epitope on gp34wt protein comprising amino acids 74-179aa)
Group III: mtrp.05/06 (recognize an epitope on gp34wt protein comprising amino acids 74-144aa)
Group IV: wt.07/08
Group V: wt.09/10/11/12
Group VI: wt13/14 (at least showed weak binding in immunoprecipitation experiments, western blots and in higher conc. in ELISA)
Group VII: wt.15

Furthermore, the data indicate that anti-gp34wt moAbs recognized structural epitopes lacking in the gp34mtrp protein, which is also impaired for oligomerization and lacks Fc-binding.

The above experiments serve as characterization of the epitopes on gp34 and gp68.Moreover, it shows that the combined usage of at least one mAb against gp34 and at least one mAb against gp68 may result in superior effects in the preferred uses as disclosed in this application.

### Proof of Concept: Selected Anti-gp68 moAbs Restore gp68-Mediated Inhibition of Human FcγRIII Activation

To fight against invading pathogens like viruses or bacteria our immune system, branched into the innate and the adaptive part, is equipped with powerful immune functions.

Interactions of IgG with Fc-γ-Receptors (FcγRs), expressed on many immune cells, are essential for opsonization, antibody dependent cellular phagocytosis (ADCP, e.g. by macrophages, dendritic cells, neutrophils) and antibody-dependent cellular cytotoxicity (ADCC) by natural killer cells (NK-cells). Most immune cells express either one specific Fc receptor or a combination of several of them. As soon as those molecules specifically bind to the IgG-Fc-part on target cells, this opsonization results in powerful antiviral effector responses, leading to the elimination of infected cells from the human body and control of pathogen replication. Accordingly, immune cells such as NK-cells and macrophages, naturally would help to control infection and dissemination e.g. of viruses.

In the case of certain herpesviruses including human cytomegalovirus (HCMV), a member of the herpesvirus family, such immune responses seem to be inefficient. To establish a successful infection as well as a lifelong persistence in the presence of immune responses like antibodies, HCMV deploys an arsenal of immune evasion molecules that allow reactivation from latency and support also superinfection. Among these immune evasines are HCMV encoded Fcy receptors (vFcγRs) which antagonize IgG-mediated effector functions of host Fcγ receptors. Four of such Fcγ receptors have been identified and characterized in HCMV, namely gp34/RL11, gp68/UL119-118, gp95/RL12 and gpRL13/RL13.

The existence of those viral vFcγRs might explain why herpesviruses such as HCMV are readily able to superinfect immune individuals and to reactivate and spread quickly unimpressed by the presence of immune IgG.

Hence, we have documented that the HCMV-encoded viral FcγR gp68 is able to antagonize IgG-mediated activation of human FcγRs e.g. of human FcγRIII/CD16 (Corrales-Aguilar et al., 2014; Kolb et al., 2021), which is the principal FcγR-receptor found on human NK-cells.

As a proof of principle, we examined in another experiment the potential of the anti-gp68 moAbs not only to block binding between the Fc-binding molecule gp68 and human IgG (as shown before: all anti-gp68 moAbs can efficiently block binding between gp68 and human IgG) but moreover to have the potential to restore human FcγRIII-activation. To this end we took advantage of our reporter cell activation assay (BW-Assay; Corrales-Aguilar et al. 2013). The reporter cell assay is based on mouse BW 5147 thymoma cells stably transduced to express individual human FcγRs as CD3-ζ chain chimeras where the FcγR ectodomain is fused to a cytoplasmic signaling module of the T-cell receptor complex, the CD3zeta chain. Upon FcγR-mediated binding to an antigen-antibody complex, reporter cells are activated and secrete mouse IL2 (mIL2) into cell culture supernatants. mIL2-levels thus indicate host FcγR-activation. In presence of HCMV vFcγRs like gp68, mIL2 levels are reduced due to antagonization by gp68. In brief, HEK293T cells were co-transfected with one plasmid encoding for gp68-CD4 (the gp68 transmembrane domain and cytoplasmic tail were exchanged by human CD4, abolishing internalization of gp68 which in turn remains surface exposed) and one plasmid encoding for human HER2 as a model antigen. 24 hours post transfection, cells were opsonized with indicated anti-gp68 moAbs (10 μg/100 μl/well) or controls for overnight. Next day, Herceptin antibody was added (1 μg/50 μl/well) for 1 hour at 37° C. Afterwards, medium from cells was completely removed before reporter cells, expressing human FcγRIII, were added (100.000 cells/in 200 μl/well) overnight. Supernatants, containing mIL2 were further analyzed by ELISA.

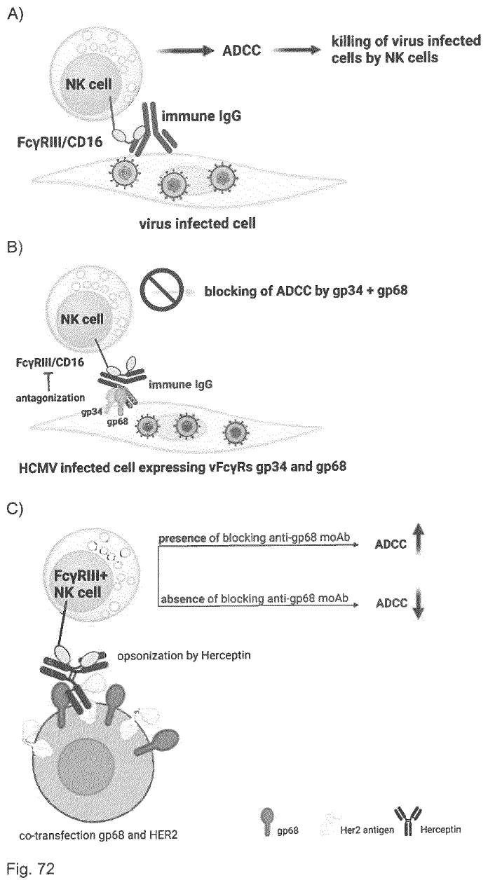

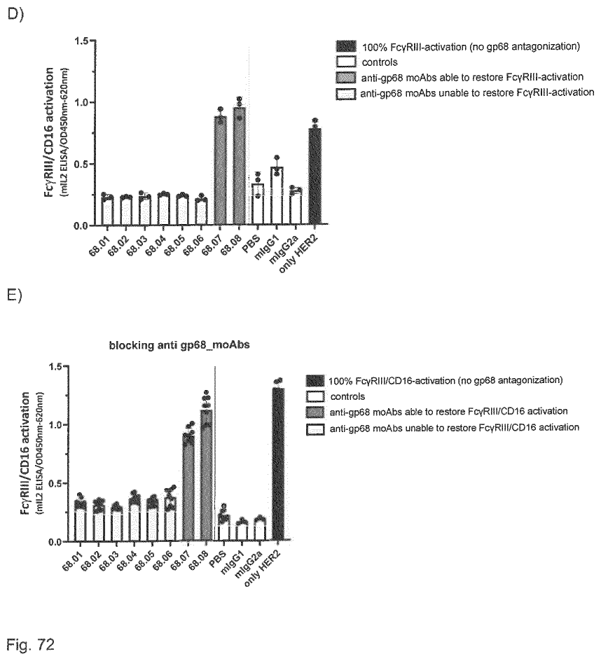

The mechanism of IgG-mediated FcγRIII/CD16-activation (A), the antagonization of IgG-mediated FcγRIII/CD16-activation by gp68 (B) and the assay principle (C) is illustrated in FIG. 72. Results of this FcγRIII-activation in presence of gp68 +/− potential blocking anti-gp68 moAbs are shown for n=1 experiment (FIG. 72D) and n=3 experiments (FIG. 72E).

FIG. 72 shows Anti-gp68 moAbs and their potential to block gp68-antagonization of human FcγRIII-activation and restoring FcγRIII receptor function. HEK293T cells were co-transfected with plasmids encoding for gp68-CD4 and HER2 antigen. One day after transfection cells were opsonized with indicated anti-gp68 moAbs (10 μg/well) or controls overnight. Anti-HER2 antibody Herceptin was added (1 μg/ml) for 1hour. Next, medium was removed before 100.000 BW-FcγRIII reporter cells were added. Next day supernatants were analyzed for mIL2 by ELISA. The mechanism of IgG-mediated FcγRIII/CD16-activation (FIG. 72A), the antagonization of IgG-mediated FcγRIII/CD16-activation by gp68 (FIG. 72B) and the experimental set up is illustrated (FIG. 72C). Levels of human FcγRIII-activation are shown for one (FIG. 72D) or 3 independent experiments (FIG. 72E). Figures were illustrated using BioRender license (created with BioRender.com).

In presence of anti-gp68 moAbs 68.01-68.06 the vFcγR gp68 is still capable to antagonized FcγRIII-activation and to similar extent when pre-incubated with PBS only or non-relevant mouse IgG1 or mouse IgG2a isotype ctrl. However, the function of gp68 pre-bound by anti-gp68 moAbs 68.07 and 68.08 is completely neutralized and FcγRIII-activation is restored to similar levels as without gp68 transfection (only HER2 antigen control, see FIGS. 72D and E).

In conclusion, anti-gp68 moAbs 68.07 and 68.08 are effective to neutralize gp68-antagonization and restore human FcγRIII/CD16-activation, which in turn will result in the killing of virus infected cells by natural killer cells.

### Proof of Concept for Anti-gp34 moAbs to Interfere with gp34 Antagonization of Human FcγRIII Activation and Restore gp34-Mediated Inhibition of Human FcγRIII Activation

We have documented that the HCMV-encoded viral FcγR gp34 is efficient to antagonize IgG-mediated activation of human FcγRs e.g. of human FcγRIII/CD16 (Corrales-Aguilar et al., 2014; Kolb et al., 2021), which is the principal FcγR-receptor found on human NK-cells.

As a proof of principle, the potential of the anti-gp34 moAbs to block the gp34-mediated antagonization of human FcγRIII-activation was tested. To this end we took advantage of the reporter cell activation assay. Due to the fact, that gp34wt itself binds to mouse IgG1, and to lesser extent to mouse IgG2a, ficin-generated Fab2-fragments of selected anti-gp34 moAbs were generated and applied. In brief, full length moAbs were treated with ficin (ThermoFisher Scientific: mouse IgG Fab2-preparation kit) according to the manufacturer's instruction, leading to digestion of the Fc-tail. Following digestion, samples are column-purified to remove remaining Fc-fragments or cleavage products thereof. By this means, Fab2-fragment of anti-vFcγR antibodies were successfully generated.

The FcγRIII-activation assay was performed in the following way:

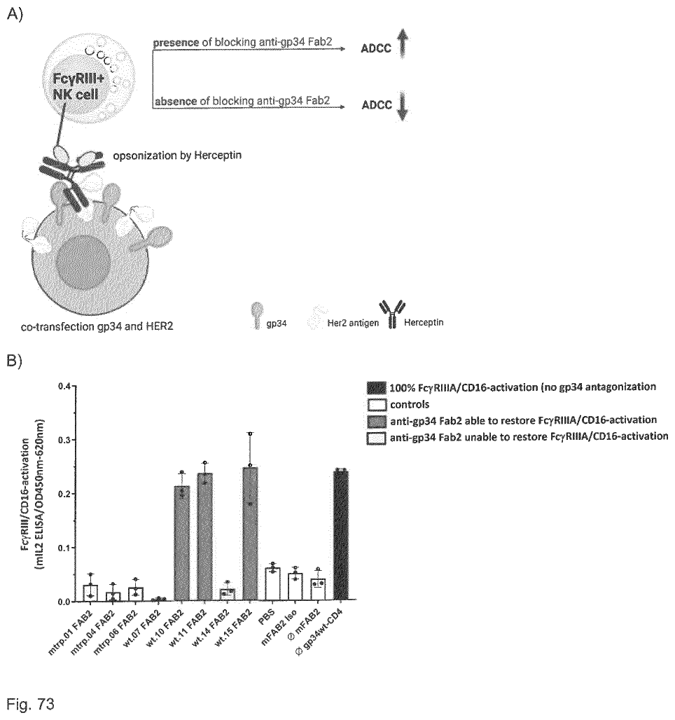

HEK293T cells were co-transfected with one plasmid encoding for gp34-CD4 (CD4 tail, abolishing internalization of gp34, which in turn remains surface exposed) and one plasmid encoding for human HER2 antigen. 24 hours post transfection, cells were opsonized with indicated mouse Fab2-fragments of our anti-gp34 moAbs (10 μg/100 μl/well) or controls overnight. Next day, Herceptin antibody was added (1 μg/50 μl/well) for 1 hour at 37° C. Afterwards, medium from cells was completely removed before reporter cells expressing human FcγRIII were added (100.000 cells/in 200 μl/well) overnight. Supernatants were analyzed by mIL2-sandwich ELISA. Results of FcγRIII-activation in presence of gp34wt +/− potential blocking anti-gp34 mouse Fab2-fragments are shown in FIG. 73.

FIG. 73 shows a Fab2 fragments of anti-gp34 moAbs and their potential to block gp34-antagonization of human FcγRIII-activation. HEK293T cells were co-transfected with plasmids encoding for gp34-CD4 and HER2 antigen. One day after transfection cells were opsonized with indicated Fab2-fragments of selected anti-gp34 moAbs (10 μg/well) or controls overnight. Anti-HER2 antibody Herceptin was added (1 μg/ml) for 1hour. After incubation time, medium was removed before 100.000 BW-FcγRIII reporter cells/well were added. Next day supernatants were analyzed in mIL2-sandwich ELISA (A). Levels of human FcγRIII-activation are shown in triplicates (B). Figures were illustrated using BioRender license (created with BioRender.com).

In presence of anti-gp34 Fab2-fragments derived from mtrp.01/mtrp.04/mtrp.06/wt.07/wt.14 as well as control Fab2-fragments or in absence of any fab2-fragment the vFcγR gp34 is still efficiently able to antagonize FcγRIII-activation to a similar extend as after pre-incubation with PBS only or non-relevant mouse Fab2 isotype ctrl. However, gp34-CD4 function is completely neutralized when gp34 is pre-bound by anti-gp34wt Fab2-fragments derived from wt.10, wt.11 and wt.15, i.e. FcγRIII-activation is restored to similar levels as without gp34 co-transfection (no gp34wt-CD4 co-transfection, only HER2 antigen).

In conclusion, Fab2-fragments derived from anti-gp34 wt.10, wt.11 and wt.15 are efficiently neutralizing gp34-antagonization and restore human FcγRIII-activation. This may serve as evidence that the antibodies according to the invention prevent HCMV infections.

### Proof of Concept for Anti-gp34 moAbs to Interference with gp34 Mediated Inhibition of IgG⁺-B-Cell Plasmablast Formation

Activation of IgG⁺ memory B cells by cognate antigen leads to differentiation and expansion of so-called CD38⁺CD27⁺ plasmablasts. These immune cells are characterized by the secretion of high affinity IgG antibodies during secondary immune responses. We found that gp34 antagonizes plasmablast formation resulting in B-cells that fail to secrete immunoglobulins. The inhibition is not limited to IgG but includes also IgM and IgA. Moreover, our data demonstrate that due to crosslinking by soluble gp34 the IgG+ BCR is only ‘suboptimally’ activated. The consequence of this gp34-induced ‘suboptimal’ stimuli leads unresponsive or anergic B-cells. As a result, such cells were unable to i) proliferate, ii) differentiate into plasmablasts and iii) secrete immunoglobulins in the presence of T cell help (CD40L and IL21). These findings now shed light on IgG⁺ B cells as a novel target for gp34 Also, this shows for the first time that gp34 represents a powerful immune modulator regulating the activation and maturation of switched memory B cells. By this mechanism, gp34, which poorly and ‘suboptimally’ activates memory B cells triggers an inefficient and ineffective humoral immune response, presumably essential during HCMV reactivation from latency.

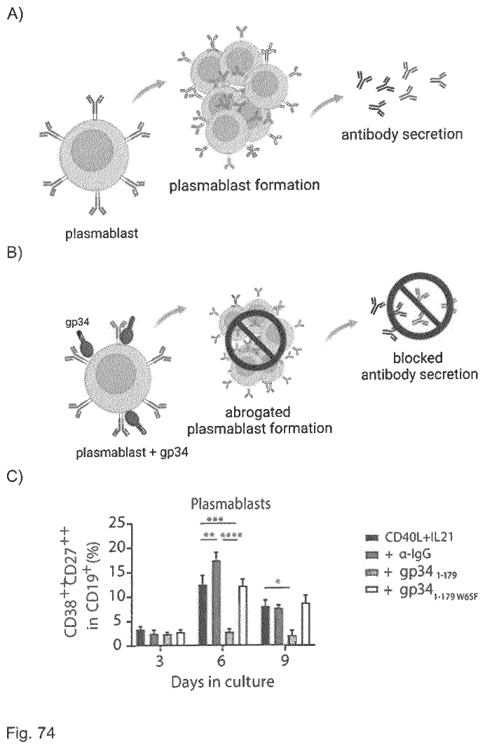

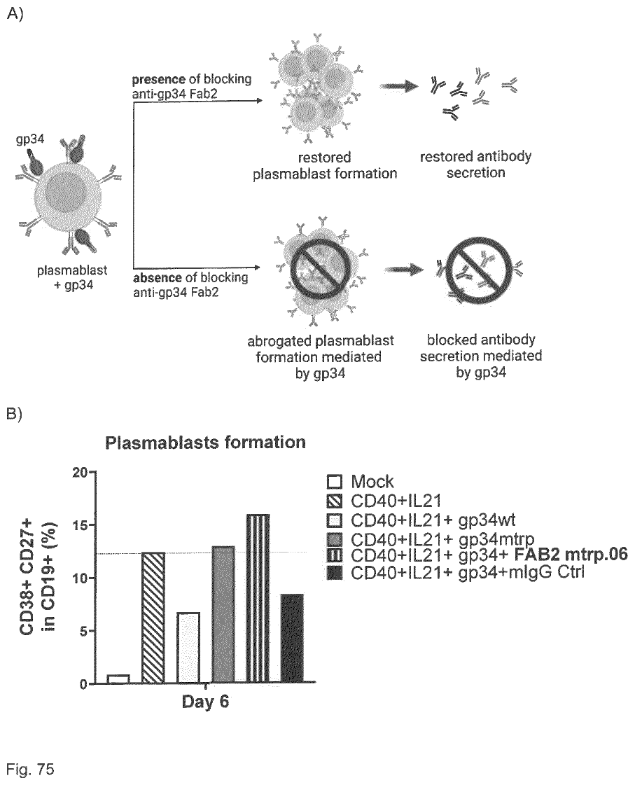

Importantly, it was demonstrated that moAbs targeting gp34 can rescue plasmablast formation in the presence of gp34 (FIG. 74). Therefore, the administration of moAbs targeting gp34 can potentially rescue B cell activity in HCMV infected individuals.

To study the impact of sgp34wt on effector functions of such IgG⁺ B cells, plasmablast formation in CD40L/IL21 stimulated B cells in the presence of sgp34wt (gp34₁₋₁₇₉) or its non-Fcγ-binding variant was assessed over a nine-day period. CD40L/IL21 acts as a T cell stimulus or the second stimulus needed for successful plasmablasts formation. 3.0×10⁵ cells/mL B cells were seeded and rested for at least 2 hrs at 37° C. The cells were stimulated with CD40L and IL21 in the presence of 10 μg/ml of α-IgG, gp34₁₋₁₇₉, and gp34_{1-179W65F} for 9 days. On days 3, 6 and 9 the supernatants were collected and stored at −20° C. and the cells analyzed for plasmablasts.

The principle of plasmablast formation and antibody secretion (A) as well as the abrogation of plasmablast formation and inhibition of antibody secretion by gp34 is illustrated in FIGS. 74(A) and (B). It was demonstrated that the cells treated with sgp34wt in the presence of CD40L/IL21 were not proliferating anymore, compared to sgp34mtrp addition (Fc-impaired gp34 point mutant with amino acid exchange at position 65 W→F) and controls (FIG. 74, C). In conclusion, we show that gp34wt inhibits plasmablast formation.

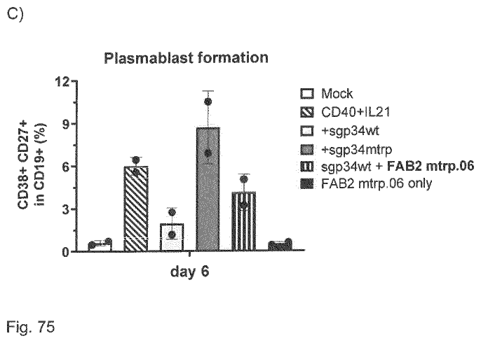

FIG. 75 is an sgp34wt abrogates plasmablast formation of memory IgG+ B cells. In-vitro stimulation of B cells showing percentage of CD38⁺⁺CD27⁺⁺ plasmablasts in the live/CD19⁺ gate over culture period. The principle of plasmablast formation and antibody secretion (A) as well as the abrogation of plasmablast formation and inhibition of antibody secretion by gp34 is illustrated (B). All cells were stimulated by CD40L and IL21. Stimulated B-cells were incubated in addition either with 10 μg/ml of sgp34wt (gp34₁₋₁₇₉), sgp34mtrp (gp34_{1-179W65F}) or α-IgG and percentage of plasmablast formation was measured by ELISA (FIG. 75C). sgp34wt, but not its Fc-impaired point-mutant sgp34mtrp, abrogates plasmablast formation of memory IgG⁺ B-cells. Figures were illustrated using BioRender license (created with BioRender.com).

Next, as a proof of concept selected Fab-2 fragments of anti-gp34 moAbs were tested regarding their potential to restore plasmablast formation even in the presence of functional sgp34wt molecules (see illustrated in FIG. 75A). Experiments were performed as before. In brief, freshly isolated PBMCs from healthy donors were left untreated or stimulated with CD40L and IL21 in absence or presence of sgp34wt, sgp34mtrp (Fc-impaired point mutant), sgp34wt pre-incubated for 1 hour with anti-gp34 moAb derived Fab2 fragment (10μg/ml) or controls. As before, on day 6 of culture, plasmablast formation (CD38⁺⁺CD27⁺⁺ plasmablasts in the live/CD19 gate) was measured by ELISA. Representative data are shown for one donor (B) or two independent B-cell donors (C).

FIG. 75 is a Fab2 of anti-gp34 mtrp.06 blocks sgp34wt-mediated inhibition of plasmablasts formation. In-vitro stimulation of B cells showing percentage of CD38⁺⁺CD27⁺⁺ plasmablasts in the live/CD19 gate on day 6 of co-culture. Cells were left untreated or stimulated by CD40L and IL21. Stimulated B-cells were incubated in addition either with 10 μg/ml of sgp34wt, sgp34mtrp, sgp34wt pre-incubated with Fab2-fragment of 10 μg/ml anti-gp34 mtrp.06 or fab2-fragment of mtrp.06 alone and percentage of plasmablast formation was measured by ELISA. sgp34wt, but not its Fc-impaired point-mutant sgp34mtrp, abrogates plasmablast formation of memory IgG⁺ B-cells. However, in presence of fab2-fragment of anti-gp34 mtrp.06 plasmablast formation is completely or at least partially restored (donor-dependent). A model of our working hypothesis is illustrated (A). Results are shown for one individual donor (B) or for another experiment with two independent donors (C). Figures were illustrated using BioRender license (created with BioRender.com).

In conclusion, the Fab2-fragment of anti-gp34 mtrp.06 blocks sgp34wt-mediated inhibition of plasmablasts formation and restores plasmablast formation.

### Proof of Concept for Anti-gp34 and Anti-gp68 moAbs to Interfere with vFcγRs gp34 and gp68 Mediated FcRn-Dependent Transcytosis of HCMV Virions

In healthy individuals, primary infection with HCMV is asymptomatic. In immunosuppressed individuals, such as transplant recipients and HIV-infected patients, HCMV infection or reactivation is associated with increased morbidity and mortality. Most importantly, human cytomegalovirus is the most common viral infection acquired in-utero, resulting in miscarriage of the infected fetus and lifelong neurological disorders in newborns (Ludwig and Hengel, 2009).

Congenital HCMV infection (cCMV) is one of the most devastating complications during pregnancy with thousands of mothers and children annually suffering from the medical and psychological consequences of this disease worldwide. As there is no vaccine available and antiviral chemotherapeutics against HCMV are not safe for administration during pregnancy, the development of Intravenous Immunoglobulin Therapy (IVIg)-based intervention strategies has been at the center of many clinical studies aiming for the prevention or treatment of cCMV.

Using a novel self-developed experimental platform, we demonstrate that HCMV virions undergo rapid transport via transcytosis across syncytiotrophoblast cells of the placenta barrier in an IgG-dependent manner before intact and infectious particles are actively delivered at the basolateral surface of the cell. This transport is mediated by the neonatal Fc receptor (FcRn), which is also tasked with the transport of IgG from mother to fetus to build up passive maternal immunity. Transcytosis is most efficient in the presence of non-neutralizing human IgG but also neutralizing human IgG.

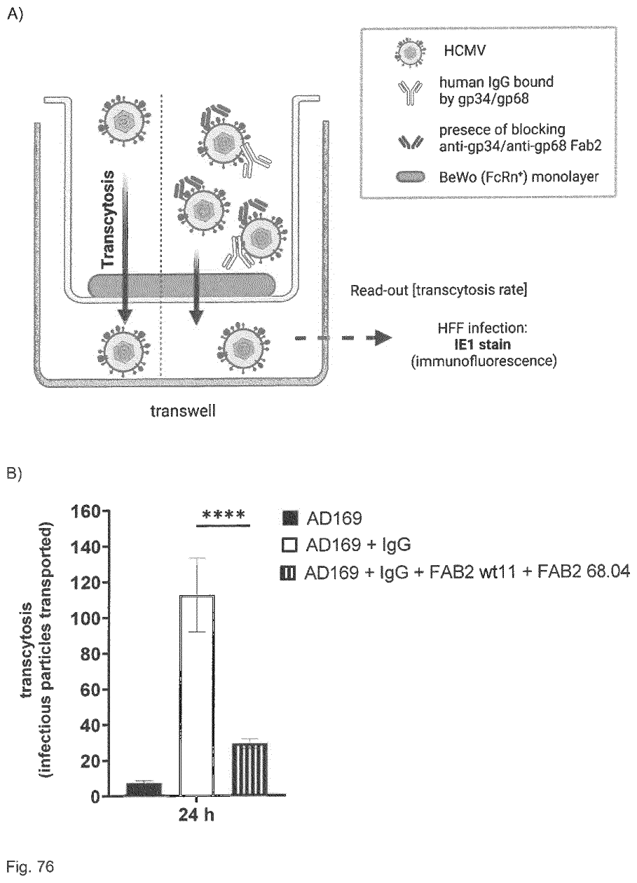

As gp34 and gp68 are found in the virion envelope and bind to IgG, we hypothesized that they are able to exploit FcRn mediated diaplacental transmission to achieve virion migration across the human placenta. Accordingly, as a proof-of-principle approach, we tested if moAbs targeting gp34 and gp68 can be used to limit virion transcytosis across the human placenta and the human syncytiotrophoblast in particular (FIG. 76). The experimental set-up was as follows: 40.000 BeWo b30 cells (expressing FcRn) were seeded into transwells. This placental cell layer is used as a model to study the barrier for maternal-fetal exchange of, e.g., IgG antibodies. The assay principle is illustrated in FIG. 76A.

Dense BeWo b30 cell layers were incubated with HCMV AD169 virions (gp34⁺ and gp68⁺) in absence or presence of a recombinant human IgG (Ofatumumab, therapeutic humanized IgG1 anti-CD20 moAb), or in presence of Ofatumumab and two selected anti-vFcγR moAbs (Fab2-fragments of one anti-gp34 and one anti-gp68 moAb). As a read-out for HCMV trancytosis bottom chamber samples were tested for transported infectious HCMV particles by detection of HCMV immediate early 1 protein in human foreskin fibroblasts cells (HFF, permissive for HCMV infection) using fluorescence microscopy. In absence of human IgG, no transmission of HCMV AD169 virions is detectable (transcytosis of infectious particles), whereas in presence of Ofatumumab representing human IgG, HCMV virions undergo transcytosed. However, IgG-mediated transcytosis of HCMV virions pre-incubated with Fab2-fragments of one anti-gp34 (wt 11) and one anti-gp68 moAbs (gp68.04) is efficiently prevented (FIG. 76, B).

FIG. 76 shows that anti-gp34/gp68 Fab2 fragments prevent gp34/gp68-mediated transcytosis. The assay principle is illustrated (FIG. 76A). 40.000 BeWo b30 were seeded in Corning Transwell™ Polycarbonate permeable supports (3 μm pore size) and incubated at MOI=1 with HCMV AD169 virions+human IgG (Ofatumumab)+/−anti HCMV vFcγR Fab2 fragments as indicated (FIG. 76B). Virions were pre-incubated with IgG in HBSS pH6 for 30 min before they were added to the BeWo b30 monolayer. Monolayer integrity was determined via transepithelial/transendothelial electrical resistance (TEER) whereby the electrical resistance across a cellular monolayer is measured. Monolayer formation was reached at TEER >400 Ohms/0.33 cm². Bottom chamber samples were tested for infectious particles via titration on HFF cells and anti-IE1 stain detection using fluorescence microscopy. 2 independent experiments are shown. Error bars=range. 2-way ANOVA (Tukey). Figures were illustrated using BioRender license (created with BioRender.com).

In summary, a combination of anti-gp34 and anti-gp68 moAbs (Fab2-fragments tested) has the potential to efficiently prevent gp34/gp68-mediated and FcRn-dependent transcytosis of HCMV virions.

1. An antibody or antigen binding fragment thereof which binds to the extracellular domain of an Fc-gamma-binding glycoprotein of human cytomegalovirus, whereby the glycoprotein is gp34 or gp68.

2. The antibody or antigen binding fragment thereof according to claim 1 wherein the Fc-gamma-binding glycoprotein is gp34.

3. The antibody or antigen binding fragment thereof according to claim 2 which binds to an extracellular domain of glycoprotein gp34 having SEQ ID NO:1.

4. The antibody or antigen binding fragment thereof according to claim 2 which binds to a mutated extracellular domain of glycoprotein gp34 having SEQ ID NO:2.

5. The antibody or antigen binding fragment thereof according to claim 1 which blocks binding of glycoprotein gp34 to an Fc part of immunoglobulin gamma or to an IgG B cell receptor.

6. The antibody or antigen binding fragment thereof according to claim 1 which blocks binding of glycoprotein gp34 to C-reactive protein.

7. The antibody or antigen binding fragment thereof according to claim 6 whereby the blocking of glycoprotein gp34 to C-reactive protein restores the function of C-reactive protein.

8. The antibody or antigen binding fragment thereof according to claim 1 which blocks binding of glycoprotein gp34 to a neonatal Fc receptor.

9. The antibody or antigen binding fragment thereof according to claim 8 wherein the antibody blocking the binding of glycoprotein gp34 to the neonatal Fc receptor restores the natural function of the neonatal Fc receptor.

10. The antibody or antigen binding fragment thereof according to claim 1 wherein the Fc-gamma-binding glycoprotein is gp68.

11. The antibody or antigen binding fragment thereof according to claim 10 which binds to the extracellular domain of glycoprotein gp68 (SEQ ID NO:3).

12. The antibody or antigen binding fragment thereof according to claim 11 which blocks the binding of glycoprotein gp68 to an Fc part of immunoglobulin gamma.

13. A humanized antibody or antigen binding fragment thereof which is derived from the antibody according to claim 1.

14. The humanized antibody or antigen binding fragment thereof according to claim 13 having the CDRs of an antibody according to claim 1.

15. A method of detecting the presence of human cytomegalovirus comprising the steps of:
reacting a biological sample derived from a potentially infected subject with an antibody or antigen binding fragment thereof according to claim 1, whereby the antibodies or antigen binding fragments thereof are labeled; and
quantitatively or qualitatively identifying a signal emitted by the label when subsequently unbound antibodies or antigen binding fragments thereof are separated from the sample, whereby the signal correlates with the presence or amount of Fc-gamma-binding glycoprotein in the sample.

16. A method of treating or preventing an HCMV infection in an subject in need thereof, said method comprising the step of administering an antibody or antigen binding fragment thereof according to claim 1 to said subject. 17. The method according to claim 16 characterized in that at least one antibody against gp34 and at least one antibody against gp68 are used in combination.

18. The method according to claim 16, wherein administration of said antibody or antigen binding fragment to said subject prevents an intra uterine infection of a fetus during pregnancy.

19. The antibody or antigen binding fragment thereof according to claim 9 wherein the antibody blocking the binding of glycoprotein gp34 to the neonatal Fc receptor prevents HCMV virion transcytosis and diaplacental transport through the neonatal Fc receptor.


SEQUENCE LISTING (73 sequences)

SEQ ID NO: 1 - protein, 182 aa, Artificial Sequence
  gp34wt
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQYTGDVEAFYLIIHPRSFCRALETRRCFYPGPGRVVVTDSQEADRAIISDLKRQWSGKPIPNPLLGLDSTRTGHHHHHH

SEQ ID NO: 2 - protein, 202 aa, Artificial Sequence
  sol gp34 mtrp
MQTYSTPLTLVIVTSLFLFTTQGSSSNAVEPTKKPLKLANYRATCEDRTRTLVTRLNTSHHSVVFQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSGKPIPNPLLGLDSTRTGHHHHHH

SEQ ID NO: 3 - protein, 315 aa, Artificial Sequence
  sol gp68
MCSVLAIALVVALLGDMHPGVKSSTTSAVTSPSNTTVTSTTSISTSNNVTSAVTTTVQTSTSSASTSVIATTQKEGHLYTVNCEASYSHDQVSLNATCKVILLNNTKNPDILSVTCYARTDCKGPFTQVGYLSAFPPDNEGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFEDGKPIPNPLLGLDSTRTGHHHHHH

SEQ ID NO: 4 - DNA, 30 bp, Artificial Sequence
  primer
cgcgctcgagatgcagacctacagcacccc

SEQ ID NO: 5 - DNA, 97 bp, Artificial Sequence
  primer
cgcgggatcctcaatggtgatggtgatgatgaccggtacgcgtagaatcgagaccgaggagagggttagggataggcttaccggaccactggcgttt

SEQ ID NO: 6 - DNA, 34 bp, Artificial Sequence
  primer
gcgcctcgagatgcggctgggcagtcctggactg

SEQ ID NO: 7 - DNA, 94 bp, Artificial Sequence
  primer
gcgcggatcctcaatggtgatggtgatgatgaccggtacgcgtagaatcgagaccgaggagagggttagggataggcttacccgtggccgcgtt

SEQ ID NO: 8 - DNA, 30 bp, Artificial Sequence; identical to SEQ ID NO: 4
  oligo used for amplification

SEQ ID NO: 9 - DNA, 97 bp, Artificial Sequence; identical to SEQ ID NO: 5
  oligo used for amplification

SEQ ID NO: 10 - DNA, 21 bp, Artificial Sequence
  oligo used for amplification
cgcgctcgagatgtgttccgt

SEQ ID NO: 11 - DNA, 97 bp, Artificial Sequence
  oligo used for amplification
cgcgggatcctcaatggtgatggtgatgatgaccggtacgcgtagaatcgagaccgaggagagggttagggataggcttaccgtcctcgaacagcgg

SEQ ID NO: 12 - DNA, 34 bp, Artificial Sequence; identical to SEQ ID NO: 6
  oligo used for amplification

SEQ ID NO: 13 - DNA, 94 bp, Artificial Sequence; identical to SEQ ID NO: 7
  oligo used for amplification

SEQ ID NO: 14 - protein, 145 aa, Artificial Sequence
  gp34 mtrp.01 heavy chain
MGILPSPGMPALLSLVSLLSVLLMGCVAQVKLEESGAELVKPGASVKLSCTGSGFNIKDIYMDWVKQRPEQGLEWIGRIDPANGNSKYDPKFQGKATITVDTSSNTAYLQLSSLTSEDTAVYYCASYYGSSYNYWGQGTTLTVSS

SEQ ID NO: 15 - protein, 149 aa, Artificial Sequence
  mtrp.01 light chain
MGILPSPGMPALLSLVSLLSVLLMGCVADIVITQTPPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELKRADAAPTVS

SEQ ID NO: 16 - protein, 145 aa, Artificial Sequence
  mtrp.01 heavy chain
MGILPSPGMPALLSLVSLLSVLLMGCVAQVKLEQSGAELVKPGASVKLSCTGSGFNIKDIYMDWVKQRPEQGLEWIGRIDPANGNSKYDPKFQGKATITVDTSSNTAYLQLSSLTSEDTAVYYCASYYGSSYNYWGQGTTLTVSS

SEQ ID NO: 17 - protein, 149 aa, Artificial Sequence; identical to SEQ ID NO: 15
  mtrp.02, light chain

SEQ ID NO: 18 - protein, 143 aa, Artificial Sequence
  mtrp.03heavy chain
MGILPSPGMPALLSLVSLLSVLLMGCVAEVQLEESGTVLARPGASVKMSCKASGYTFTSYWMHWVKQRPGQGLEWIGAIYPGNSDTGYNQKFKGKARLTAVTSTSTAYLELSSLTDEDSAVYFCTRLGYGDYWGQGTTLTVSS

SEQ ID NO: 19 - protein, 135 aa, Artificial Sequence
  mtrp.03,light chain
MGILPSPGMPALLSLVSLLSVLLMGCVADIVLTQTPSSMYASLGERVTITCKASQDINSYLTWFQQKPGKSPKTLIYRANRLVDGVPSRFSGSGSGQDYSLTINSLDYEDMGIYYCLQYDEFPPTFGGGTKLEIK

SEQ ID NO: 20 - protein, 143 aa, Artificial Sequence
  mtrp.04, heavy chain
MGILPSPGMPALLSLVSLLSVLLMGCVAEVKLVQSGTVLARPGASVKMSCKASGYTFTSYWMHWVKQRPGQGLEWIGAIYPGNSDTGYNQKFKGKARLTAVTSTSTAYLELSSLTDEDSAVYFCTRLGYGDYWGQGTTLTVSS

SEQ ID NO: 21 - protein, 135 aa, Artificial Sequence
  mtrp.04, light chain
MGILPSPGMPALLSLVSLLSVLLMGCVADIVMTQTPSSMYASLGERVTITCKASQDINSYLTWFQQKPGKSPKTLIYRANRLVDGVPSRFSGSGSGQDYSLTINSLDYEDMGIYYCLQYDEFPPTFGGGTKLEIK

SEQ ID NO: 22 - protein, 145 aa, Artificial Sequence
  mtrp.05, heavy chain
MGILPSPGMPALLSLVSLLSVLLMGCVAEVKLLQSGPEVKKPGETVKISCKASGYTFTNYGMSWLRQAPGKGLKWMGWINTYTGEPTYADDFKGRFAFSLETSASTAYFQINNLKDEDTTTYFCARRAFGGLDYWGQGTSVTVSS

SEQ ID NO: 23 - protein, 140 aa, Artificial Sequence
  mtrp.05, light chain
MGILPSPGMPALLSLVSLLSVLLMGCVADIVMTQSPLSLPVSLGDQASISCRSSQSIVYTNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 24 - protein, 145 aa, Artificial Sequence
  mtrp.06 heavy chain
MGILPSPGMPALLSLVSLLSVLLMGCVAEVQLLQSGPEVKKPGETVKISCKASGYTFTNYGMSWLRQAPGKGLKWMGWINTYTGEPTYADDFKGRFAFSLETSASTAYFQINNLKNEDTTTYFCARRAFGGLDYWGQGTSVTVSS

SEQ ID NO: 25 - protein, 140 aa, Artificial Sequence
  mtrp.06 , light chain
MGILPSPGMPALLSLVSLLSVLLMGCVADIVLTQSPLSLPVSLGDQASISCRSSQSIVYTNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 26 - protein, 149 aa, Artificial Sequence
  gp34 wt 07, heavy chain
MGILPSPGMPALLSLVSLLSVLLMGCVAQVKLEESGPELVKTGASVKISCKASGYSFTSYQIYWVKQSHGKSLEWIGHISCYNGATSYNQKFKDKATFTVDTSSSTAYMQFNSLTSEDSAVYFCARYDYAPDYYAMDYWGQGTSVTVSS

SEQ ID NO: 27 - protein, 139 aa, Artificial Sequence
  wt.07, light chain
MGILPSPGMPALLSLVSLLSVLLMGCVADIVLTQTPASLAVSLGQRATISCRASESVDSYGNSFMHWYQQKPGQPPKLLIYLASNLESGVPARFSGSGSRTDFTLTIDPVEAEDAAIYYCQQNNEDPLTFGGGTKLELK

SEQ ID NO: 28 - protein, 149 aa, Artificial Sequence
  gp34 wt 08, heavy chain
MGILPSPGMPALLSLVSLLSVLLMGCVAEVQLEQSGPELVKTGASVKISCKASGYSFTSYQIYWVKQSHGKSLEWIGHISCYNGATSYNQKFKDKATFTVDTSSSTAYMQFNSLTSEDSAVYFCARYDYAPDYYAMDYWGQGTSVTVSS

SEQ ID NO: 29 - protein, 139 aa, Artificial Sequence
  wt.08, light chain
MGILPSPGMPALLSLVSLLSVLLMGCVADIVMTQTPASLAVSLGQRATISCRASESVDSYGNSFMHWYQQKPGQPPKLLIYLASNLESGVPARFSGSGSRTDFTLTIDPVEAEDAAIYYCQQNNEDPLTFGGGTKLELK

SEQ ID NO: 30 - protein, 143 aa, Artificial Sequence
  gp34 wt 09,heavy chain
MGILPSPGMPALLSLVSLLSVLLMGCVAEVQLEQSGPELVKPGASVKMSCKASGYTFTRPYIHWVKQRPGQGLEWIGWIYPGDGITKYNEKFKGKITLTADKSSSAAYMLLSSLTSEDSAIYFCASFTTPVYWGQGTSVTVSS

SEQ ID NO: 31 - protein, 140 aa, Artificial Sequence
  wt.09, light chain
MGILPSPGMPALLSLVSLLSVLLMGCVADIVMTQTPLTLSVTIGQPASISCKSSQSLLDSDGKTYLNWLFQRPGQSPKRLIYLVSKLDSGVPDRFTGSGSGTDFTLKISRVEAEDLGVYYCWQGTHFPQTFGGGTKLEIK

SEQ ID NO: 32 - protein, 143 aa, Artificial Sequence
  gp34  wt.10,heavy chain
MGILPSPGMPALLSLVSLLSVLLMGCVAEVKLEQSGPELVKPGASVKMSCKASGYTFTRPYIHWVKQRPGQGLEWIGWIYPGDGITKYNEKFKGKITLTADKSSSAAYMLLSSLTSEDSAIYFCASFTTPVYWGQGTSVTVSS

SEQ ID NO: 33 - protein, 140 aa, Artificial Sequence
  wt.10,light chain
MGILPSPGMPALLSLVSLLSVLLMGCVADIVLTQTPLTLSVTIGQPASISCESSQSLLDSDGKTYLNWLFQRPGQSPKRLIYLVSKLDSGVPDRFTGSGSGTDFTLKICRVEAEDLGVYYCWQGTHFPQTFGGGTKLEIK

SEQ ID NO: 34 - protein, 143 aa, Artificial Sequence
  gp34 wi.11,heavy chain
MGILPSPGMPALLSLVSLLSVLLMGCVAQVQLEESGPELVKPGASVKMSCKASGYTVTRSYIHWVKQRPGQGLEWIGWIYPGDGSTKYNEKFKGKTTLTVDKSSSTAYMLLSSLTSEDSAIYFCASFTTPVYWGQGTSVTVSS

SEQ ID NO: 35 - protein, 140 aa, Artificial Sequence
  wt.11
MGILPSPGMPALLSLVSLLSVLLMGCVADIVLTQSPLTLSVTIGQPASISCKSSQSLLDSDGETYLNWLLQRPGQSPKRLIYLVSKLDSGVPDRFTGSGSGTDFTLKISRVEAEDLGVYYCWQGTHFPQTFGGGTKLEIK

SEQ ID NO: 36 - protein, 143 aa, Artificial Sequence
  wt.12, heavy chain
MGILPSPGMPALLSLVSLLSVLLMGCVAEVKLQESGPELVKPGASVKMSCKASGYTVTRSYIHWVKQRPGQGLEWIGWIYPGDGSTKYNEKFKGKTTLTVDKSSSTAYMLLSSLTSEDSAIYFCASFTTPVYWGQGTSVTVSS

SEQ ID NO: 37 - protein, 140 aa, Artificial Sequence
  wt.12, light chain
MGILPSPGMPALLSLVSLLSVLLMGCVADIVLTQTPLTLSVTIGQPASISCKSSQSLLDSDGETYLNWLLQRPGQSPKRLIYLVSKLDSGVPDRFTGSGSGTDFTLKISRVEAEDLGVYYCWQGTHFPQTFGGGTKLEIK

SEQ ID NO: 38 - protein, 145 aa, Artificial Sequence
  wt13,heavy chain
MGILPSPGMPALLSLVSLLSVLLMGCVAEVQLVESGPELKKPGETVKISCKASGYAFTNYGMNWVKQTPGKGLKWMGWINTYTGKPTYADDFKGRFAFSLETSASSAYLQINNLKNEDTATYFCARRRGNSFDYWGQGTTLTVSS

SEQ ID NO: 39 - protein, 141 aa, Artificial Sequence
  wt 13, light chain
MGILPSPGMPALLSLVSLLSVLLMGCVADIVLTQSPSSLTVTAGEKVTMSCKSSQSLLNSGNQENCLTWYQQKPGQPPRLLIYWASTRESGVPDRFTGSGSGTDFTLTISSVQAEDLAVYYCQNDYTYPLTFGAGTKLELK

SEQ ID NO: 40 - protein, 145 aa, Artificial Sequence
  gp34,wt14, heavy chain
MGILPSPGMPALLSLVSLLSVLLMGCVAQVKLLQSGPELKKPGETVKISCKASGYAFTNYGMNWVKQTPGKGLKWMGWINTYTGKPTYADDFKGRFAFSLETSASSAYLQINNLKNEDTATYFCARRRGNSFDYWGQGTTLTVSS

SEQ ID NO: 41 - protein, 141 aa, Artificial Sequence
  wt14, light chain
MGILPSPGMPALLSLVSLLSVLLMGCVADIVITQSPSSLTVTAGEKVTMSCKSSQSLLNSGNQENCLTWYQQKPGQPPRLLIYWASTRESGVPDRFTGSGSGTDFTLTISSVQAEDLAVYYCQNDYTYPLTFGAGTKLELK

SEQ ID NO: 42 - protein, 143 aa, Artificial Sequence
  gp34,wt.15
MGILPSPGMPALLSLVSLLSVLLMGCVAQVKLQQSGPELVKPGASVKMSCKASGYTFRSYYIHWVKQRPGQGLEWIGWNNPGDGTAKYNEKFKGKTTLTADKSSSTAYMLLSSLTSEDSAIYFCASFNTPDYWGQGTSVTVSS

SEQ ID NO: 43 - protein, 140 aa, Artificial Sequence; identical to SEQ ID NO: 35
  wt.15,light chain

SEQ ID NO: 44 - protein, 146 aa, Artificial Sequence
  gp68, 68.01, heavy chain
MGILPSPGMPALLSLVSLLSVLLMGCVAEVKLLESGPELVKPGSSVKISCKASGYSFADHIILWVKQSHGKSLEWIGNINPYSGNTIYNLNFKGKATLTVDKSSSTAYMQLNSLTSEDSAVYYCARIEGYYSMDYWGQGTSVTVSS

SEQ ID NO: 45 - protein, 140 aa, Artificial Sequence
  68.01, light chain
MGILPSPGMPALLSLVSLLSVLLMGCVADIVMTQTPLSLPVSLGDQASISCRSSQSIVHSNGNTYLEWYLQKPGQSPKVLIYKVSKRFSGVPDRFSGSGSGTDFTLKISRVEAEDMGVYYCFQGSHVPLTFGAGTKLELK

SEQ ID NO: 46 - protein, 146 aa, Artificial Sequence
  gp68 clone 68.02, heavy chain
MGILPSPGMPALLSLVSLLSVLLMGCVAEVKLLQSGPELVKPGSSVKISCKASGYSFADHIILWVKQSHGKSLEWIGNINPYSGNTIYNLNFKGKATLTVDKSSSTAYMQLNSLTSEDSAVYYCARIEGYYSMDYWGQGTSVTVSS

SEQ ID NO: 47 - protein, 140 aa, Artificial Sequence
  68.02, light chain
MGILPSPGMPALLSLVSLLSVLLMGCVADIVLTQTPLSLPVSLGDQASISCRSSQSIVHSNGNTYLEWYLQKPGQSPKVLIYKVSKRFSGVPDRFSGSGSGTDFTLKISRVEAEDMGVYYCFQGSHVPLTFGAGTKLELK

SEQ ID NO: 48 - protein, 131 aa, Artificial Sequence
  gp68, clone 68.03, heavy chain
MGCVAEVKLEESGPEVKKPGETVKISCKASGYTFTNYGMSWLRQAPGKGLKWMGWINTYTGEPTYADDFKGRFAFSLETSASTAYFQINNLKDEDTTTYFCARRAFGGLDYWGQGTSVTVFSAKTTPPSVY

SEQ ID NO: 49 - protein, 149 aa, Artificial Sequence
  68.03, light chain
MGILPSPGMPALLSLVSLLSVLLMGCVADIVLTQTPLSLPVSLGDQASISCRSSQSIVHSNGNTYLEWYLQKPGQSPKVLIYKVSKRFSGVPDRFSGSGSETDFTLKISRVEAEDMGVYYCFQGSHVPLTFGAGTKLELKRADAAPTVS

SEQ ID NO: 50 - protein, 154 aa, Artificial Sequence
  gp68,clone 68.04, heavy chain
MGILPSPGMPALLSLVSLLSVLLMGCVAEVQLQESGPEVKKPGETVKISCKASGYTFTNYGMSWLRQAPGKGLKWMGWINTYTGEPTYADDFKGRFAFSLETSASTAYFQINNLKNEDTTTYFCARRAFGGLDYWGQGTSVTVSSAKTTPPSVY

SEQ ID NO: 51 - protein, 149 aa, Artificial Sequence
  68.04, light chain
MGILPSPGMPALLSLVSLLSVLLMGCVADIVLTQTPLSLPVSLGDQASISCRSSQNIVHSNGNTYLEWYLQKPGQSPKLLIYKVSNRFSGVPDRFSGSGSGTDFTLKISRVEAEDLGVYYCFQGSHVPYTFGGGTKLEVKRADAAPTVS

SEQ ID NO: 52 - protein, 158 aa, Artificial Sequence
  gp68, clone 68.05, heavy chain
MGILPSPGMPALLSLVSLLSVLLMGCVAQVKLEESGAELMKPGASVKISCTATGYTFSGYWIEWVKQRPGHGLEWIGEILPGSGGTNYNEKFKGKATFTADTSSNTAYMQLSSLTSEDSAVYYCARGIHYFGYYYFDYWGQGTTLTVSSAKTTPPSVY

SEQ ID NO: 53 - protein, 144 aa, Artificial Sequence
  68.05, light chain
MGILPSPGMPALLSLVSLLSVLLMGCVADIVLTQTPSSLSASLGDRVTISCRASQDISNYLNWYQLKPDGTVKLLIYYTSRLHSGVPSRFSGSGSGTDYSLTISNLEQEDIATYFCQQGNTLPPTFGGGTKLEIKRADAAPTVS

SEQ ID NO: 54 - protein, 158 aa, Artificial Sequence
  gp68, clone 68.06,heavy chain
MGILPSPGMPALLSLVSLLSVLLMGCVAQVKLEQSGAELMKPGASVKISCTATGYTFSGYWIEWVKQRPGHGLEWIGEILPGSGGTNYNEKFKGKATFTADTSSNTAYMQLSSLTSEDSAVYYCARGIHYFGYYYFDYWGQGTTLTVSSAKTTPPSVY

SEQ ID NO: 55 - protein, 144 aa, Artificial Sequence
  68.06, light chain
MGILPSPGMPALLSLVSLLSVLLMGCVADIVLTQSPSSLSASLGDRVTISCRASQDISNYLNWYQLKPDGTVKLLIYYTSRLHSGVPSRFSGSGSGTDYSLTINNLEQEDIATYFCQQGNTLPPTFGGGTKLEIKRADAAPTVS

SEQ ID NO: 56 - protein, 159 aa, Artificial Sequence
  gp68 clone 68.07, heavy chain
MGILPSPGMPALLSLVSLLSVLLMGCVAEVKLVQSGGGLVQPGGSLRLSCATSGFTFSDFYMEWVRQPPGKRLEWIAASRNKANDYATEYSASVKGRFIVSRDTSQSILYLQMNALRAEDTAIYYCARDAGDYWGYFNVWGAGTTVTVSSAKTTPPSVY

SEQ ID NO: 57 - protein, 150 aa, Artificial Sequence
  68.07, light chain
MGILPSPGMPALLSLVSLLSVLLMGCVADIVMTQTPSSLGVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELKRADAAPTVS

SEQ ID NO: 58 - protein, 159 aa, Artificial Sequence
  gp68, clone 68.08, heavy chain
MGILPSPGMPALLSLVSLLSVLLMGCVAQVQLVESGGGLVQPGGSLRLSCATSGFTFSDFYMEWVRQPPGKRLEWIAASRNKANDYATEYSASVKGRFIVSRDTSQSILYLQMNALRAEDTAIYYCARDAGDYWGYFNVWGAGTTVTVSSAKTTPPSVY

SEQ ID NO: 59 - protein, 150 aa, Artificial Sequence
  68.08, light chain
MGILPSPGMPALLSLVSLLSVLLMGCVADIVITQSPSSLGVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELKRADAAPTVS

SEQ ID NO: 60 - protein, 17 aa, Artificial Sequence
  V5 tag
GKPIPNPLLGLDSTRTG

SEQ ID NO: 61 - protein, 6 aa, Artificial Sequence
  poly - His tag
HHHHHH

SEQ ID NO: 62 - protein, 5 aa, Artificial Sequence
  pOPINVL
CVAGT

SEQ ID NO: 63 - protein, 9 aa, Artificial Sequence
  mouse heavy chain
AKTTPPSVY

SEQ ID NO: 64 - DNA, 15 bp, Artificial Sequence
  pOPINVH
tgcgtagctggtacc

SEQ ID NO: 65 - DNA, 30 bp, Artificial Sequence
  Mouse IGG 1 heavy chain
ggcgccaaaacgacacccccatctgtctat

SEQ ID NO: 66 - DNA, 15 bp, Artificial Sequence
  pOPINVH
acgcatcgaccatgg

SEQ ID NO: 67 - DNA, 30 bp, Artificial Sequence
  mouse Igg1 heavy chain
ccgcggttttgctgtgggggtagacagata

SEQ ID NO: 68 - protein, 5 aa, Artificial Sequence
  pOPInVL
CVAGT

SEQ ID NO: 69 - protein, 9 aa, Artificial Sequence
  mouse kappa light chain
RADAAPTVS

SEQ ID NO: 70 - DNA, 15 bp, Artificial Sequence
  pOPINVL
tgcgtagctggtacc

SEQ ID NO: 71 - DNA, 32 bp, Artificial Sequence
  mouse kappa light chain
gagctcgggctgatgctgcaccaactgtatcc

SEQ ID NO: 72 - DNA, 15 bp, Artificial Sequence
  pOPINVL
acgcatcgaccatgg

SEQ ID NO: 73 - DNA, 32 bp, Artificial Sequence
  mouse kappa light chain
ctcgagcccgactacgacgtggttgacatagg